(19) United States
(12) Patent Application Publication
(10) Pub. No.: US 2023/0121031 A1
(43) Pub. Date: Apr. 20, 2023

(54) MASKED FLUOROGENIC COMPOUNDS AND METHODS OF USING THE SAME

(71) Applicant: Rutgers, The State University of New Jersey, New Brunswick, NJ (US)

(72) Inventors: Enver Izgu, Piscataway, NJ (US); Tushar Aggarwal, Piscataway, NJ (US)

(73) Assignee: Rutgers, The State University of New Jersey, New Brunswick, NJ (US)

(21) Appl. No.: 17/898,995

(22) Filed: Aug. 30, 2022

Related U.S. Application Data
(60) Provisional application No. 63/238,894, filed on Aug. 31, 2021.

(51) Int. Cl. A61K 49/00; A61K 47/55; C12N 15/115; G01N 33/543; A61K 47/54; G01N 21/64

(52) U.S. Cl. CPC A61K 49/0021; A61K 49/0056; A61K 49/0052; A61K 47/55; C12N 15/115; G01N 33/54353; A61K 47/549; G01N 33/54386; G01N 21/6428; C12N 2310/16; C12N 2310/351; G01N 2021/6439

(57) ABSTRACT

In one aspect, the present disclosure relates to a masked fluorogenic compound comprising a small molecule protecting group that can be cleaved following a reaction with a biomarker. In some embodiments, cleavage of the small molecule protecting group provides a fluorogenic ligand that binds to an aptamer, leading to fluorescence emission. In another aspect, the present disclosure relates to a method of detecting a disease or a disorder in a subject and/or in a biological sample from the subject.

### CROSS-REFERENCE TO RELATED APPLICATIONS

The present application claims priority under 35 U.S.C. § 119(e) to U.S. Provisional Patent Application No. 63/238,894, filed Aug. 31, 2021, which is incorporated herein by reference in its entirety.

### STATEMENT REGARDING FEDERALLY SPONSORED RESEARCH

This invention was made with government support under grant number 1R21EB029548-01 awarded by the National Institutes of Health. The government has certain rights in the invention.

### BACKGROUND OF THE INVENTION

Nucleic acids have been fundamental to the advancement of biotechnology and medicine. Their ability to self-organize through sequence-dependent hybridization and folding have been exploited to construct a myriad of technologies ranging from nanometer-scale machines to macromolecular agents for cellular imaging. However, nucleic acid tools have had limited applicability towards investigating cellular anomalies, such as metabolic dysfunction or stress. Our current understanding of aberrant metabolic pathways at the molecular level remains limited.

Nucleic acids could be engineered to study various intracellular environments, as they can self-organize—through either Watson-Crick or non-Watson-Crick base pairing—to provide molecular architectures with profound three-dimensional complexity through which discrete functionality can arise. Aptamers are functional oligonucleotides that bind a specific target (ligand), which is typically a small organic molecule. These functional oligonucleotide sequences can be found in nature as part of gene regulation elements located in the untranslated regions of mRNAs or can be discovered in the laboratory by in vitro selection from a random pool of oligonucleotides. Certain aptamers can bind and substantially increase the fluorescence quantum yield (Φ_{f}) of fluorophores that are poorly fluorescent in their unbound state. Aptamers offer distinct advantages over imaging approaches that solely depend on small molecules. For example, cells can be engineered to co-express aptamers to visualize and understand cellular fates of an RNA of interest through its localization and trafficking.

There is now considerable interest in developing approaches in which aptamers can be used for fluorescence imaging of molecules in living cells. The expansion of methods that use aptamer-small molecule interactions as practical and adaptable platforms could lead to advanced detection strategies for biomarkers associated with human diseases and infectious specimens. Despite their immense potential, the natural biophysical properties of aptamers have limited both their development as configurable imaging tools and their implementation in translational science. Each aptamer sequence is highly specific to a ligand with a distinct molecular structure, which renders the aptamers unable to detect most biomarkers, especially inorganic metabolites or enzymes. Furthermore, each distinct molecular target requires in vitro selection of a new aptamer sequence, rendering the development of multiplexed biosensing remarkably challenging. Consequently, aptamers have had minimal biochemical application as tools to elucidate aberrant cellular conditions.

There remains a need in the art for designer small molecules to enable a single RNA aptamer to detect multiple, unique, and structurally diverse disease-associated biomarkers, including, but not limited to, inorganic molecules and enzymes. The present invention satisfies these unmet needs.

### BRIEF SUMMARY OF THE INVENTION

In one aspect, the present invention provides a compound of Formula (II), or a salt, solvate, stereoisomer, or geometric isomer thereof:

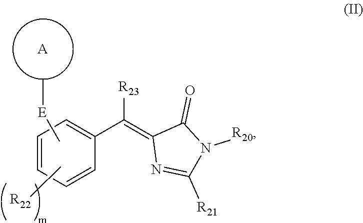

wherein R₂₀, R₂₁, R₂₂, R₂₃, A, E, and m are defined within the scope of the present invention.

In certain embodiments, the compound of Formula (II) is a compound of Formula (IIa), or a salt, solvate, stereoisomer, or geometric isomer thereof:

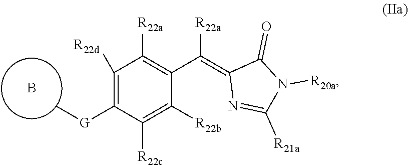

wherein R₂₀ₐ, R₂₁ₐ, R₂₂ₐ, R_{22b}, R_{22c}, R_{22d}, R₂₃ₐ, B, and G are defined within the scope of the present invention.

In certain embodiments, the compound of Formula (II) and/or Formula (IIa) is selected from the group consisting of

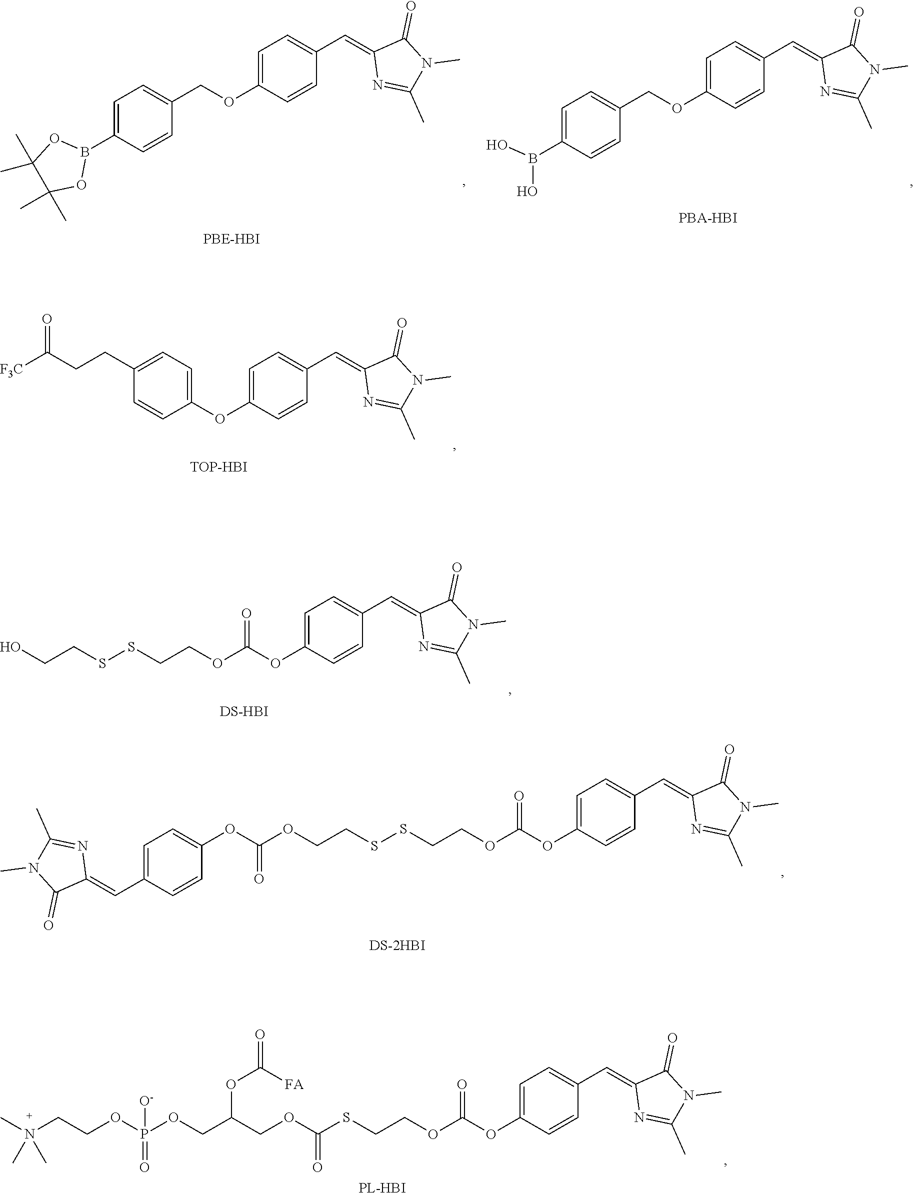

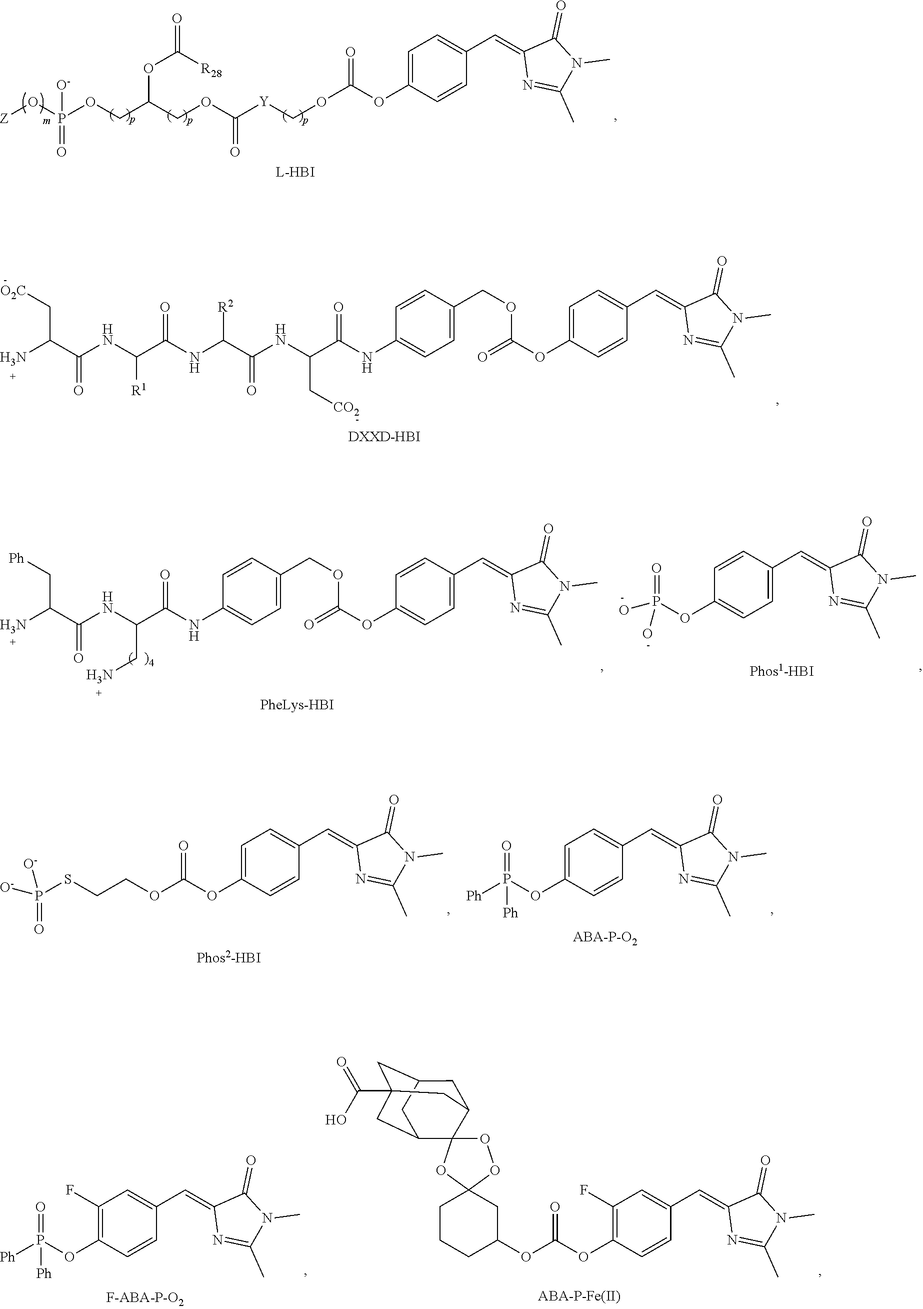

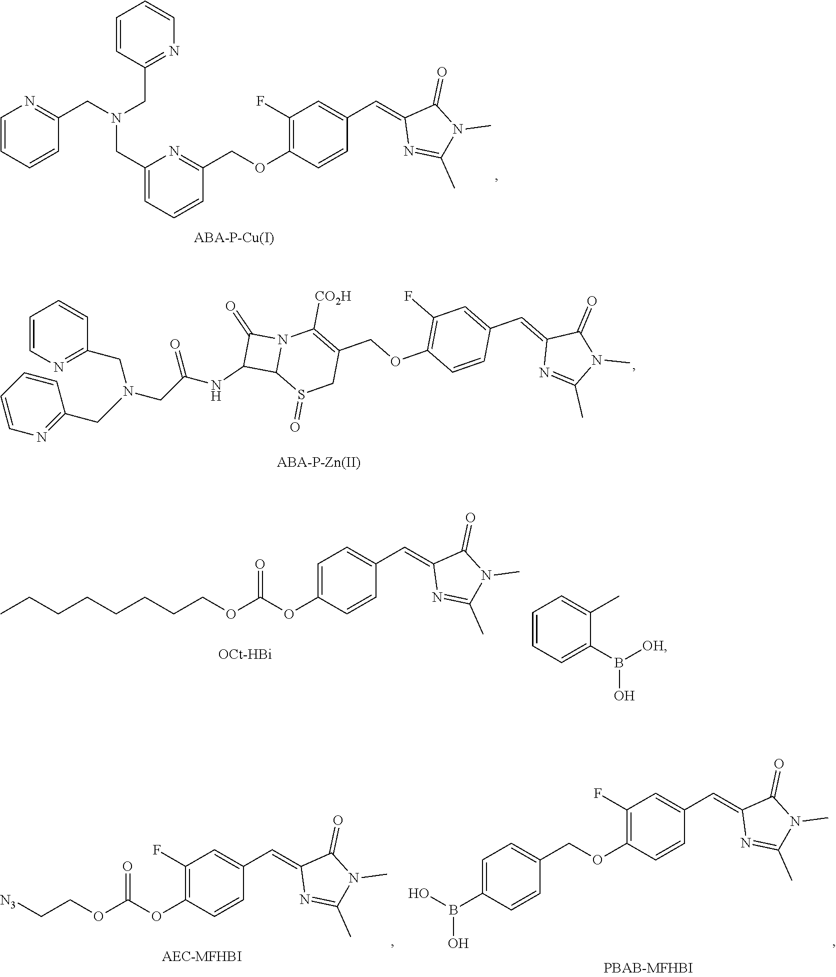

and combinations thereof;
wherein R¹ and R² are each independently selected from the group consisting of hydrogen, CH₃, CH₂—OH, and CH(OH)—CH₃.

In certain embodiments, the compound of Formula (II) reacts with a biomarker to provide a compound of Formula (I), or a salt, solvate, stereoisomer, or geometric isomer thereof:

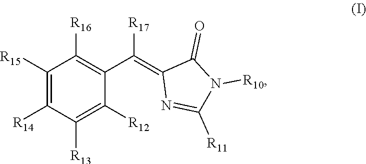

wherein R₁₀, R₁₁, R₁₂, R₁₃, R₁₄, R₁₅, R₁₆, and R₁₇ are defined within the scope of the present invention.

In certain embodiments, the biomarker is selected from the group consisting of hydrogen peroxide (H₂O₂), dimethylsulfide (H₂S), superoxide (O₂⁻), peroxynitrite (ONOO⁻), glutathione, hepatic lipase, cathepsin B, the caspase family, alkaline phosphatase, Cu(I), Fe(II), and Zn(II), and combinations thereof.

In another aspect, the present invention provides a method of detecting a disease or a disorder in a subject in need thereof. In certain embodiments, the method comprises expressing an RNA sequence comprising an RNA aptamer in the subject. In certain embodiments, the method comprises administering to the subject a compound of Formula (II), or a salt, solvate, stereoisomer, or geometric isomer thereof:

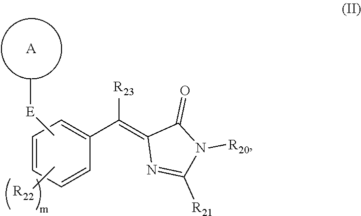

wherein R₂₀, R₂₁, R₂₂, R₂₃, A, E, and m are defined within the scope of the present invention. In certain embodiments, the method comprises detecting fluorescence emission associated with the RNA aptamer.

In certain embodiments, the RNA aptamer is selected from the group consisting of Spinach aptamer, Baby Spinach aptamer, Corn aptamer, and Broccoli aptamer.

In certain embodiments, administering to the subject a compound of Formula (II) further comprises deprotecting the compound of Formula (II) via a reaction with a biomarker, producing a fluorogenic ligand.

In certain embodiments, the biomarker is selected from the group consisting of hydrogen peroxide, dimethylsulfide, superoxide, hydroxyl radical, hydroxide anion, peroxynitrite, nitrogen dioxide, nitrosoperoxycarbonate, dinitrogen trioxide, aldehyde, glutathione, glutathione-synthesizing enzymes, lipases, cathepsin B, the caspase family, acid phosphatase, alkaline phosphatase, Cu(I), Fe(II), and Zn(II), and combinations thereof.

In certain embodiments, the fluorogenic ligand is a compound of Formula (I), or a salt, solvate, stereoisomer, or geometric isomer thereof:

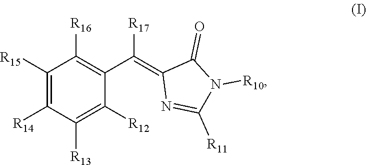

wherein R₁₀, R₁₁, R₁₂, R₁₃, R₁₄, R₁₅, R₁₆, and R₁₇ are defined within the scope of the present invention.

In certain embodiments, deprotecting the compound of Formula (II) via a reaction with a biomarker further comprises binding the fluorogenic ligand to the RNA aptamer.

In certain embodiments, binding the fluorogenic ligand to the RNA aptamer leads to fluorescence emission associated with the aptamer.

In certain embodiments, the fluorescence emission associated with the aptamer indicates that the subject has a disease or disorder associated with the biomarker.

In another aspect, the present invention provides a method of detecting a disease or a disorder in a biological sample. In certain embodiments, the method comprises providing a chip comprising a grafted RNA aptamer. In certain embodiments, the method comprises contacting the chip with a biological sample. In certain embodiments, the method comprises contacting the biological sample with a compound of Formula (II), or a salt, solvate, stereoisomer, or geometric isomer thereof:

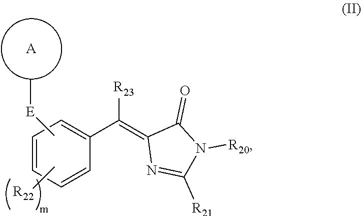

wherein R₂₀, R₂₁, R₂₂, R₂₃, A, E, and m are defined within the scope of the present invention. In certain embodiments, the method comprises rinsing the chip. In certain embodiments, the method comprises detecting fluorescence emission from the RNA aptamer.

In certain embodiments, the RNA aptamer is Spinach aptamer, Baby Spinach aptamer, Corn aptamer, or Broccoli aptamer.

In certain embodiments, contacting the biological sample with a compound of Formula (II) further comprises deprotecting the compound of Formula (II) via a reaction with a biomarker present in the biological sample, producing a fluorogenic ligand.

In certain embodiments, the biomarker is selected from the group consisting of hydrogen peroxide, dimethylsulfide, superoxide, hydroxyl radical, hydroxide anion, peroxynitrite, nitrogen dioxide, nitrosoperoxycarbonate, dinitrogen trioxide, aldehyde, glutathione, glutathione-synthesizing enzymes, lipases, cathepsin B, the caspase family, acid phosphatase, alkaline phosphatase, Cu(I), Fe(II), and Zn(II), and combinations thereof.

In certain embodiments, the fluorogenic ligand is a compound of Formula (I), or a salt, solvate, stereoisomer, or geometric isomer thereof:

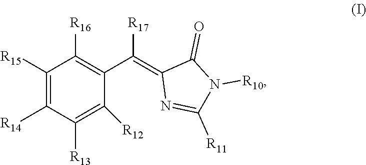

wherein R₁₀, R₁₁, R₁₂, R₁₃, R₁₄, R₁₅, R₁₆, and R₁₇ are defined within the scope of the present invention.

In certain embodiments, deprotecting the compound of Formula (II) via a reaction with a biomarker further comprises binding the fluorogenic ligand to the RNA aptamer.

In certain embodiments, binding the fluorogenic ligand to the RNA aptamer leads to fluorescence emission associated with the aptamer.

In certain embodiments, the fluorescence emission associated with the aptamer indicates that the biological sample comprises a biomarker associated with a disease or disorder.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIGS. 1A-1B depict schematics demonstrating the disclosed ABA biosensor comprising a fluorogenic ligand which has been masked using a biomarker-specific chemical modification. The modification is removed in the presence of the biomarker, providing the free ligand which then binds to the aptamer, resulting in a strong fluorescence signal. FIG. 1A depicts this process in cellulo. FIG. 1B depicts this process on a chip.

FIGS. 2A-2B provides a schematic depicting a tRNA comprising an RNA_{Spinach} aptamer which does not fluoresce until binding with DFHBI (FIG. 2A) and cellular images demonstrating the same (FIG. 2B).

FIGS. 3A-3M depict a non-limiting small molecule library with structural modifications to HBI and/or MFHBI that are reactive towards their respective biomarker. FIG. 3A depicts H₂O₂ detection: Phenyl boronic ester-masked HBI (PBE-HBI). FIG. 3B depicts ONOO⁻ detection: p-(4,4,4-Trifluoro-3-oxobutyl)phenyl-masked HBI (TOP-HBI). FIG. 3C depicts GSH detection: Disulfide-linked mono-HBI (DS-HBI) and disulfide-linked bis-HBI (DS-2HBI). FIG. 3D depicts HL detection: Phospholipidated HBI (PL-HBI). FA: Fatty alkyl. FIG. 3E depicts cathepsin B detection: PheLys-HBI. FIG. 3F depicts caspase detection: Asp-AA-AA-Asp-linked HBI (DXXD-HBI). FIG. 3G depicts ALP detection: Phosphorylated HBIs (Phos¹-HBI and Phos²-HBI). FIG. 3H depicts O₂⁻ detection: phosphinate-conjugated HBIs (e.g., ABA-P-O₂). FIG. 3I depicts Fe(II) detection: spirocyclic endoperoxide-conjugated HBIs (e.g., ABA-P-Fe(II)). FIG. 3J depicts Cu(I) detection: tris[(2-pyridylmethyl)amino] alkyl-conjugated HBIs (e.g., ABA-P-Cu(I)). FIG. 3K depicts Zn(II) detection: β-lactam thianone-conjugated HBIs (e.g., ABA-P-Zn(II)). FIG. 3L depicts SH₂ detection: azidoalkyl carbonate-conjugated HBIs (e.g., AEC-MFHBI). FIG. 3M depicts H₂O₂ detection: p-boronic acid benzyl (PBAB)-conjugated HBIs (e.g., PBAB-MFHBI).

FIGS. 4A-4E depict the effect of chemical modifications of HBI on its binding interactions with RNA. FIG. 4A is a crystal structure of DFHBI-RNA_{Spinach} (PDB: 4TS2) focusing on the key H-bonding interactions of DFHBI within the G-G-A binding pocket of RNA_{Spinach}. FIG. 4B is a Pymol view of docked HBI-RNA_{Spinach}, indicating that these interactions are conserved. FIG. 4C is a Pymol view of docked Oct-HBI-RNA_{Spinach}, showing that these interactions are disrupted. FIG. 4D is a Pymol view of a docked methyl carbonate-modified HBI-RNA_{Spinach}, showing that these interactions are disrupted. FIG. 4E depicts normalized RFUs of HBI and HBI+RNA_{BabySpinach} (gray bars) vs. Oct-HBI and Oct-HBI+RNA_{BabySpinach} (blue bars). Fluorophore (2 μM), RNA (1 μM), HEPES (pH 8, 20 mM), KCl (100 mM), and MgCl₂ (10 mM).

FIG. 5 depicts the chemical design principle of the H₂O₂-responsive aptamer ligand, PBE-HBI.

FIG. 6 depicts the ¹H-NMR investigation of the conversion of PBE-HBI to HBI using H₂O₂. PBE-HBI (1.5 mM) was dissolved in a 3:1 d₆-DMSO-H₂O (pH 8) mixture. The spectral overlay was acquired using pentafluoro-benzaldehyde (δₑₓₜ=10.28 ppm) as the external reference and the PRESAT sequence as to suppress the bulk water peak.

FIGS. 7A-7C depict H₂O₂ treatment of the aptamer systems. HBI and PBE-HBI (50 μM), RNA (1 μM RNA_{BabySpinach}), HEPES (pH 8, 50 mM), KCl (100 mM), MgCl₂ (10 mM), H₂O₂ (100 μM). FIG. 7A depict the time-dependent change in fluorescence intensity upon H₂O₂ addition. FIG. 7B depicts normalized RFUs at 0, 5, and 60 min. FIG. 7C depicts RNA_{BabySpinach} stability against H₂O₂. TBE-urea PAGE lanes: 1. Native RNA; 2. [HBI+RNA]; 3. [HBI+RNA+H₂O₂]; 4. [PBE-HBI+RNA]; 5. [PBE-HBI+RNA+H₂O₂]. For imaging, the gel was stained with SYBR gold.

FIGS. 8A-8D depict detection of H₂O₂ in E. coli utilizing PBAB-MFHBI. FIG. 8A provides a schematic showing the detection of H₂O₂ in E. coli by fluorescence of MFHBI upon cleavage from PBAB-MFHBI. FIGS. 8B-8D provide fluorescence images of E. coli expressing aptamer plasmid which have been administered MFHBI (FIG. 8B), PBA-MFHBI (FIG. 8C), and PBA-MFHBI in the presence of H₂O₂ (FIG. 8D). Dual channel: MFHBI+RNA (470/550 nm) and FM 4-64FX (510/640 nm). Scale bar: 10 μm.

FIGS. 9A-9D depict detection of H₂S in E. coli utilizing AEC-MFHBI. FIG. 9A provides a schematic showing the detection of H₂S in E. coli by fluorescence of MFHBI upon cleavage from AEC-MFHBI. FIGS. 9B-9D provide fluorescence images of E. coli expressing aptamer plasmid which have been administered MFHBI (FIG. 9B), AEC-MFHBI (FIG. 9C), and AEC-MFHBI in the presence of H₂S (FIG. 9D). Dual channel: MFHBI+RNA (470/550 nm) and FM 4-64FX (510/640 nm). Scale bar: 10 μm.

FIGS. 10A-10C depict the engineering of aptamer-grafted chips. FIG. 10A shows surface coating by mussel-inspired polymerization of catecholamines. FIG. 10B shows the aptamer-grafted chips. (i) Surface nanostructuring; (ii) photolithography; (iii) coating/grafting. *Substituted benzylidene moiety. FIG. 10C shows the capture and detection of pathogens using nanoneedle/aptamer chips.

### DETAILED DESCRIPTION OF THE INVENTION

The present disclosure provides in one aspect masked fluorogenic compounds comprising a small molecule protecting group that can be cleaved following a reaction with a biomarker. In certain embodiments, cleavage of the small molecule protecting group provides a fluorogenic ligand that binds to an aptamer. In some embodiments, binding of the fluorogenic ligand with the aptamer leads to fluorescence emission by the fluorogenic ligand. In yet other embodiments, the masked fluorogenic compounds of the disclosure can be used to detect a metabolic process, a disease, or a disorder in a subject in need thereof. In yet other embodiments, the masked fluorogenic compounds can be used to detect a metabolic process, a disease, or a disorder in a biological sample. In some embodiments, the biomarker that reacts with the masked fluorogenic compound is a biomarker that is associated with a specific disease or disorder or a class of diseases or disorders. In some embodiments, the masked fluorogenic compounds can be used to study the physiological role and concentration profile of certain biomarkers in live cells. In some embodiments, the masked fluorogenic compounds can be used to explore how biomarkers impact complex molecular networks and metabolic pathways of a cell during the initiation and/or progression of a disease.

The skilled artisan will understand that the invention is not limited to the masked fluorogenic compounds discussed herein. Further, the skilled artisan will understand that the masked fluorogenic ligands can be administered to a subject to detect the presence of any biomarker that can cleave the small molecule protecting group, providing a fluorogenic ligand. Still further, a skilled artisan will understand that any aptamer that can bind the fluorogenic ligand can be used. Still further, a skilled artisan will understand that the masked fluorogenic compounds can be administered to a subject after the subject has received a therapeutic treatment for a disease or disorder in order to detect changes in the intensity of the fluorescence emission. In some embodiments, changes in the intensity of the fluorescence emission can be used to monitor the efficacy of the treatment.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of 20% or ±10%, in certain embodiments ±5%, in certain embodiments ±1%, in certain embodiments ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

The term “biological” or “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

The term “expression” as used herein is defined as the transcription and/or translation of a particular nucleotide sequence driven by its promoter.

The term “RNA” as used herein is defined as ribonucleic acid.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆)alkyl, such as, but not limited to, ethyl, methyl, isopropyl, isobutyl, n-pentyl, n-hexyl, and cyclopropylmethyl.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one-to-six carbon alkylene chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one-to-three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, the term “halo” or “halogen” alone or as part of another substituent refers to, unless otherwise stated, a fluorine, chlorine, bromine, or iodine atom.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl, or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet other embodiments, the substituents vary in number between one and two. In yet other embodiments, the substituents are independently selected from the group consisting of C₁-C₆ alkyl, —OH, C₁-C₆ alkoxy, halo, cyano, amino, acetamido and nitro. As used herein, where a substituent is an alkyl or alkoxy group, the carbon chain may be branched, straight or cyclic.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁- C₂, C₂-C₆, C₂-C₅, C₂-C₄, C₂-C₃, C₃-C₆, C₃-C₅, C₃-C₄, C₄-C₆, C₄-C₅, and C₅- C₆ alkyl.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

### Fluorogenic Ligand

In one aspect, the present disclosure relates to a fluorogenic ligand. In certain embodiments, the fluorogenic ligand is a ligand that is capable of binding an aptamer. In some embodiments, the fluorogenic ligand fluoresces upon binding to an aptamer and has reduced or no fluorescence emission when not bound to an aptamer. In certain embodiments, the fluorogenic ligand is 4-hydroxy-benzylidene imidazolinone (HBI), or an isomer, tautomer, derivative, or salt thereof. In certain embodiments, the fluorogenic ligand is monofluoro-4-hydroxy-benzylidene imidazolinone (MFHBI) (i.e., (Z)-5-(3-fluoro-4-hydroxybenzylidene)-2,3-dimethyl-3,5-dihydro-4H-imidazol-4-one). In certain embodiments, the fluorogenic ligand is difluoro-4-hydroxy-benzylidene imidazolinone (DFHBI). In certain embodiments, the fluorogenic ligand is HBI.

In certain embodiments, the fluorogenic ligand is a compound of Formula (I), or a salt, solvate, stereoisomer, or geometric isomer thereof:

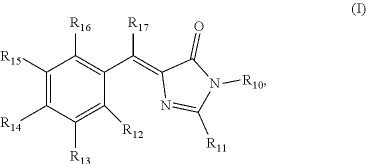

wherein:

R₁₀, R₁₁, and R₁₇ are each independently selected from the group consisting of hydrogen, deuterium, and optionally substituted C₁-C₆ alkyl;

R₁₂, R₁₃, R₁₄, R₁₅, and R₁₆ are each independently selected from the group consisting of hydrogen, deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;

each occurrence of R′ is independently selected from the group consisting of hydrogen, deuterium, hydroxy, C₁-C₆ alkyl, and C₁-C₆ alkoxy,

with the proviso that one or more of R₁₂, R₁₃, R₁₄, R₁₅, and R₁₆ is selected from the group consisting of OH, NHR′, and SH.

In certain embodiments, R₁₀ and R₁₁ are each unsubstituted C₁-C₆ alkyl. In certain embodiments, R₁₀ and R₁₁ are each methyl. In certain embodiments, R₁₀ is CF₃ and R₁₁ is methyl. In other embodiments, R₁₀ is methyl and R₁₁ is CF₃. In other embodiments, R₁₀ and R₁₁ are each CF₃.

In certain embodiments, one of R₁₂, R₁₃, R₁₄, R₁₅, and R₁₆ is hydroxy, amino, or thiol. In certain embodiments, R₁₄ is hydroxy. In certain embodiments, R₁₄ is amino. In certain embodiments, R₁₄ is thiol. In certain embodiments, R₁₃ is F. In certain embodiments, R₁₅ is F. In certain embodiments, R₁₃ is F and R₁₄ is OH. In certain embodiments, R₁₃ is F, R₁₃ is OH, and R₁₅ is F.

In certain embodiments R₁₂, R₁₃, R₁₅, and R₁₆ are each hydrogen.

In certain embodiments, R₁₇ is hydrogen.

### Masked Fluorogenic Ligand

In another aspect, the present disclosure relates to a masked fluorogenic ligand. In certain embodiments, the masked fluorogenic ligand comprises a chemical modification such that it cannot bind an aptamer. In certain embodiments, the masked fluorogenic ligand has reduced or no florescence. In some embodiments, the masked fluorogenic ligand comprises a chemically modified ligand of Formula (I). In certain embodiments, the ligand of Formula (I) is modified via the binding of a small organic molecule to the hydroxy, amino, or thiol group at R₁₂, R₁₃, R₁₄, R₁₅, or R₁₆ of Formula (I). In certain embodiments, the ligand of Formula (I) is modified via the binding of a small organic molecule to the hydroxy, amino, or thiol group at R₁₄ of Formula (I). In other embodiments, the masked fluorogenic ligand comprises chemically modified HBI. In certain embodiments, HBI is chemically modified via the binding of a small organic molecule to the phenolic oxygen. In certain embodiments, the masked fluorogenic ligand comprises chemically modified MFHBI. In certain embodiments, MFHBI is chemically modified via the binding of a small organic molecule to the phenolic oxygen. In certain embodiments, the masked fluorogenic ligand comprises chemically modified DFHBI. In certain embodiments, DFHBI is chemically modified via the binding of a small organic molecule to the phenolic oxygen. In certain embodiments, the binding is a covalent bond.

In certain embodiments, the masked fluorogenic ligand reacts with a biomarker, deprotecting the fluorogenic ligand by removing the chemical modification, and releasing the fluorogenic ligand. In some embodiments, the chemical modification is removed by cleaving the bond between the small organic molecule and a benzylic oxygen, sulfur, or nitrogen atom from the hydroxy, amino, or thiol group of Formula (I). In certain embodiments, the chemical modification is removed by cleaving the bond between the small organic molecule and the phenolic oxygen of HBI. In certain embodiments, the released fluorogenic ligand is a compound of Formula (I). In certain embodiments, the released fluorogenic ligand is HBI. In certain embodiments, the released fluorogenic ligand is MFHBI. In certain embodiments, the released fluorogenic ligand is DFHBI.

The biomarker that reacts with the masked fluorogenic ligand can be any biomarker known to a person of skill in the art. Exemplary biomarkers include, but are not limited to, reactive oxygen species (e.g., hydrogen peroxide, superoxide, hydroxyl radical, hydroxide anion), reactive nitrogen species (e.g., peroxynitrite, nitrogen dioxide, nitrosoperoxycarbonate, dinitrogen trioxide), aldehydes (e.g., formaldehyde, acetaldehyde), glutathione (GSH), glutathione-synthesizing enzymes (e.g., γ-glutamylcysteine ligase), lipases (e.g., hepatic lipase (HL)), cathepsin B, the caspase family, acid phosphatase, alkaline phosphatase (ALP), transition metals (e.g., Cu(I), Fe(II), and Zn(II)), and combinations thereof.

In certain embodiments, the masked or caged fluorogenic ligand is a compound of Formula (II), or a salt, solvate, stereoisomer, or geometric isomer thereof:

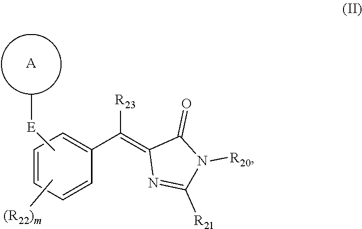

wherein:

R₂₀, R₂₁, and R₂₃ are each independently selected from the group consisting of hydrogen, deuterium, and optionally substituted C₁-C₆ alkyl;

each occurrence of R₂₂ is independently selected from the group consisting of deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;

each occurrence of R′ is independently selected from the group consisting of hydrogen, deuterium, hydroxy, C₁-C₆ alkyl, and C₁-C₆ alkoxy;

m is an integer from 0 to 4;

E is selected from the group consisting of —O—, —S—, and —NH—;

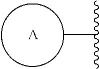

is selected from the group consisting of

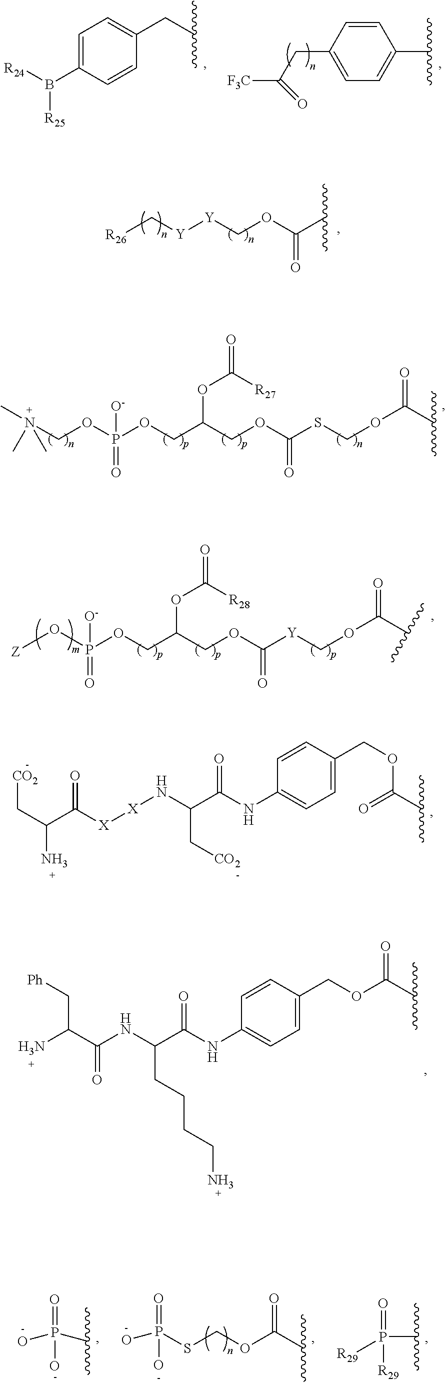

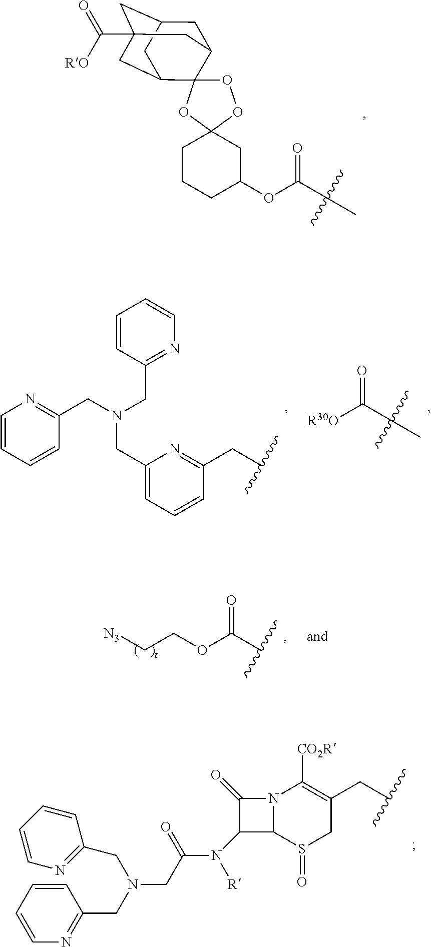

wherein:

m is 0 or 1;

each occurrence of n is independently 2 or 3;

each occurrence of p is independently 1, 2, or 3;

t is 1, 2, or 3;

R₂₄ and R₂₅ are each independently selected from the group consisting of hydroxy, C₁-C₆ alkyl, and C₁-C₆ alkoxy,
or R₂₄ and R₂₅ can combine with the atoms to which they are bound to form a 4-6 membered ring;

R₂₆ is hydroxy or

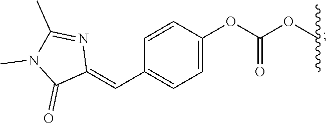 [SMILES: *OC(=O)Oc1ccc(/C=C2\N=C(C)N(C)C2=O)cc1]

R₂₇ and R₂₈ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;

each occurrence of R²⁹ is independently selected from the group consisting of optionally substituted C₁-C₆ alkyl, optionally substituted C₆-C₁₀ aryl, and optionally substituted C₂-C₁₀ heteroaryl;

R³⁰ is optionally substituted C₁-C₁₂ alkyl;

each occurrence of X is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;

Y is selected from the group consisting of —O—, —NH—, and —S—; and

Z is selected from the group consisting of choline, ethanolamine, serine, inositol, glycerol, phosphatidylcholine, lysophosphatidic acid, and glucose.

In certain embodiments, R₂₀ and R₂₁ are each C₁-C₆ alkyl. In certain embodiments R₂₀ and R₂₁ are each methyl. In certain embodiments, R₂₀ is CF₃ and R₂₁ is methyl. In other embodiments, R₂₀ is methyl and R₂₁ is CF₃. In other embodiments, R₂₀ and R₂₁ are each CF₃.

In certain embodiments, each instance of R₂₂ is hydrogen.

In certain embodiments, R₂₃ is hydrogen.

In certain embodiments, each instance of n is 2.

In certain embodiments, each instance of p is 1. In certain embodiments, two instances of p are 1 and one instance of p is 2.

In certain embodiments, R₂₄ and R₂₅ are each hydroxy.

In certain embodiments,

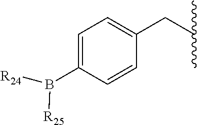

is

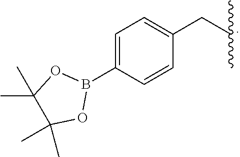 [SMILES: *Cc1ccc(B2OC(C)(C)C(C)(C)O2)cc1]

In certain embodiments, R₂₇ is a fatty alkyl. Exemplary fatty alkyl groups include, but are not limited to, the fatty alkyl structure of palmitic acid, stearic acid, oleic acid, arachidonic acid, linoleic acid, α-linolenic acid, γ-linolenic acid, and docosahexaenoic acid. In certain embodiments, R₂₇ is a steroid family lipid. Exemplary steroid family lipids, include but not limited to cholesterol, sterols, aldosterone, and cholic acid. In certain embodiments, R₂₇ is a sex hormones or androgen. Exemplary sex hormones or androgens include, but are not limited to, progesterone and testosterone.

In certain embodiments, R₂₈ is a fatty alkyl. Exemplary fatty alkyl groups include, but are not limited to, the fatty alkyl structure of palmitic acid, stearic acid, oleic acid, arachidonic acid, linoleic acid, α-linolenic acid, γ-linolenic acid, and docosahexaenoic acid. In certain embodiments, R₂₈ is a steroid family lipid. Exemplary steroid family lipids, include but not limited to cholesterol, sterols, aldosterone, and cholic acid. In certain embodiments, R₂₈ is a sex hormone or androgen. Exemplary sex hormones or androgens include, but are not limited to, progesterone and testosterone.

In certain embodiments, R₂₉ is Ph. In certain embodiments, each occurrence of R₂₉ is independently Ph.

In certain embodiments, R₃₀ is octyl.

In certain embodiments, t is 1.

In certain embodiments, each X is independently glycine, alanine, serine, or threonine.

In certain embodiments, the reaction of the masked fluorogenic ligand with a biomarker cleaves the bond between

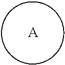

and E of the compound of Formula (II).
In certain embodiments, the compound of Formula (II) is a compound of Formula (IIa), or a salt, solvate, stereoisomer, or geometric isomer thereof:

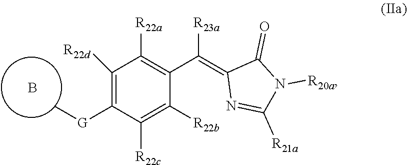

wherein:

G is selected from the group consisting of —O—, —S—, or —NH—;

R₂₀ₐ, R₂₁ₐ, and R₂₃ₐ are each independently selected from the group consisting of hydrogen, deuterium, and optionally substituted C₁-C₆ alkyl;

R^{22a}, R^{22b}, R^{22c}, and R^{22d} are each independently selected from the group consisting of hydrogen, deuterium, tritium, halogen, hydroxy, N(R″)(R″), SR″, sulfide, thiolactone, S(═O)₂OR″, S(═O)R″, cyano, C₁-C₆ alkyl, C₂-C₆ alkenyl, C₂-C₆ alkynyl, nitro, C(═O)N(R″)(R″), P(═O)(OR″)₂, PR″₃, C₆-C₁₂ aryl, C₄-C₁₀ heteroaryl, C₁-C₆ alkoxy, optionally substituted boron, optionally substituted silicon, transition metal, C(═O)OR″, and C(═O)R″;

each occurrence of R″ is independently selected from the group consisting of hydrogen, deuterium, hydroxy, C₁-C₆ alkyl, and C₁-C₆ alkoxy;

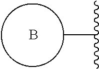

is selected from the group consisting of:

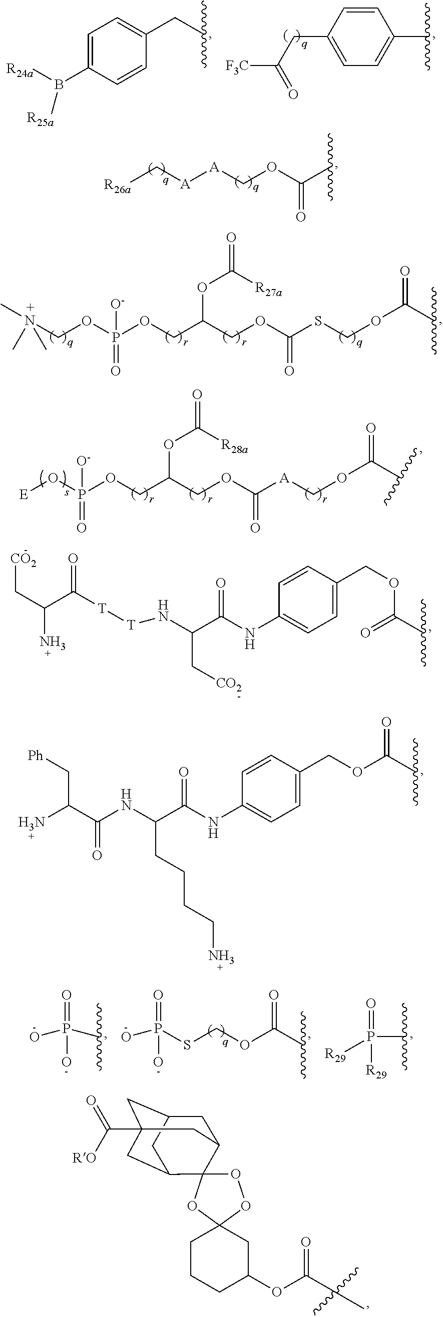

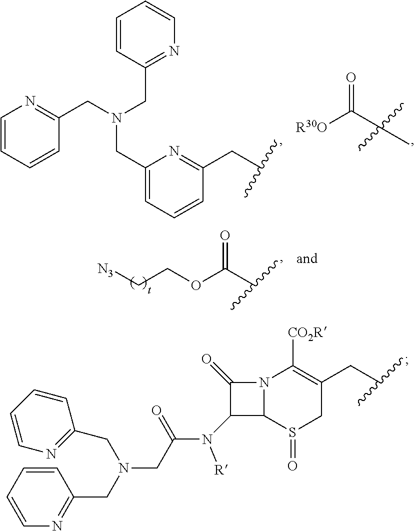

wherein:

each occurrence of q is independently 2 or 3;

each occurrence of r is independently 1, 2, or 3;

s is 0 or 1;

R₂₄ₐ and R₂₅ₐ are each independently selected from the group consisting of hydroxy, C₁-C₆ alkyl, and C₁-C₆ alkoxy,
or R₂₄ₐ and R₂₅ₐ can combine with the atoms to which they are bound to form a 4-6 membered ring;

R₂₆ₐ is hydroxy or

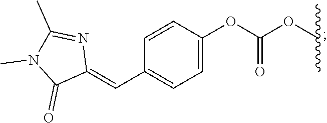 [SMILES: *OC(=O)Oc1ccc(/C=C2\N=C(C)N(C)C2=O)cc1]

R₂₇ₐ and R₂₈ₐ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;

A is selected from the group consisting of —O—, —NH—, and —S—;

E is selected from the group consisting of choline, ethanolamine, serine, inositol, glycerol, phosphatidylcholine, lysophosphatidic acid, and glucose; and

each occurrence of T is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and selenocysteine.

In certain embodiments, R₂₀ₐ and R₂₁ₐ are each C₁-C₆ alkyl. In certain embodiments R₂₀ₐ and R₂₁ₐ are each methyl. In certain embodiments, R₂₀ₐ is CF₃ and R₂₁ₐ is methyl. In other embodiments, R₂₀ₐ is methyl and R₂₁ₐ is CF₃. In other embodiments, R₂₀ₐ and R₂₁ₐ are each CF₃.

In certain embodiments, each instance of R₂₂ₐ is hydrogen.

In certain embodiments, R₂₃ₐ is hydrogen.

In certain embodiments, each instance of q is 2.

In certain embodiments, each instance of r is 1. In certain embodiments, two instances of r are 1 and one instance of r is 2.

In certain embodiments, R₂₄ₐ and R₂₅ₐ are each hydroxy.

In certain embodiments,

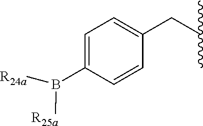

is

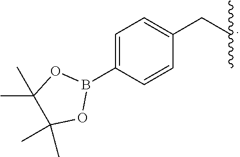 [SMILES: *Cc1ccc(B2OC(C)(C)C(C)(C)O2)cc1]

In certain embodiments, R₂₇ₐ is a fatty alkyl. Exemplary fatty alkyl groups are described elsewhere herein. In certain embodiments, R₂₇ₐ is a steroid family lipid. Exemplary steroid family lipids are described elsewhere herein. In certain embodiments, R₂₇ₐ is a sex hormone or androgen. Exemplary sex hormones or androgens are described elsewhere herein.

In certain embodiments, R₂₈ₐ is a fatty alkyl. Exemplary fatty alkyl groups are described elsewhere herein. In certain embodiments, R₂₈ₐ is a steroid family lipid. Exemplary steroid family lipids are described elsewhere herein. In certain embodiments, R₂₈ₐ is a sex hormone or androgen. Exemplary sex hormones or androgens are described elsewhere herein.

In certain embodiments, R₂₉ is Ph. In certain embodiments, each occurrence of R₂₉ is independently Ph.

In certain embodiments, R₃₀ is octyl.

In certain embodiments, t is 1.

In certain embodiments, each T is independently glycine, alanine, serine, or threonine.

In certain embodiments, the reaction of the masked fluorogenic ligand with a biomarker cleaves the bond between

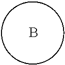

### and G of Formula (IIa).

In certain embodiments, the masked fluorogenic ligand of Formula (II) and/or Formula (IIa) is selected from the group consisting of:

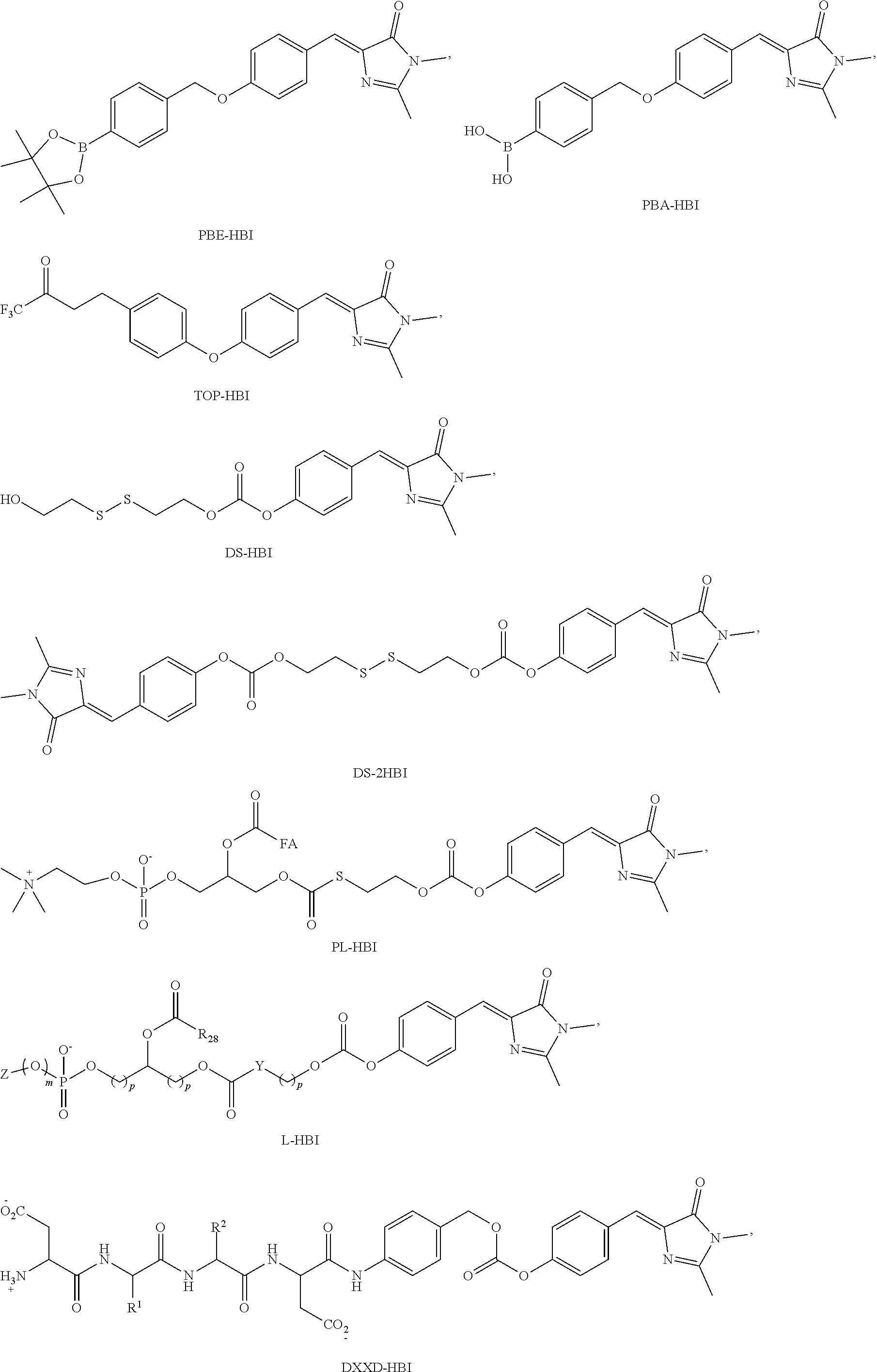

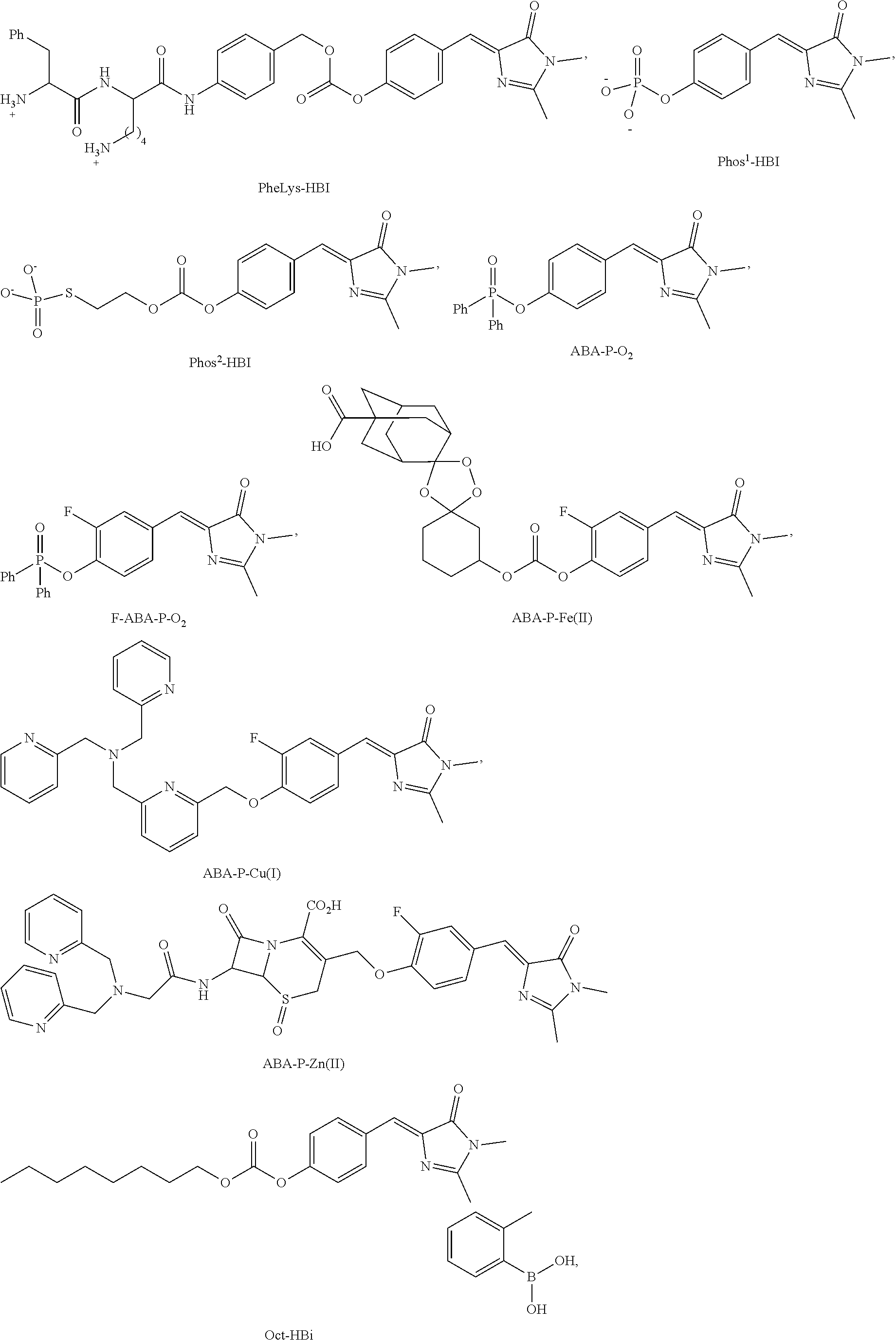

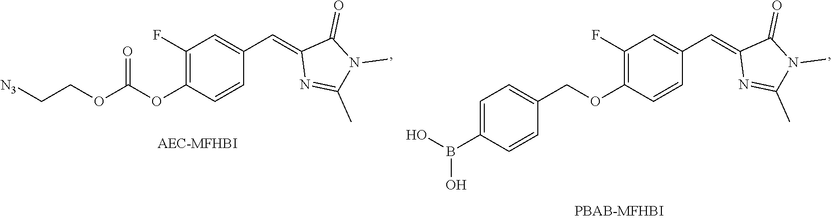 [SMILES: CC1=N/C(=C\c2ccc(OC(=O)OCCN=[N+]=[N-])c(F)c2)C(=O)N1C ; CC1=N/C(=C\c2ccc(OCc3ccc(B(O)O)cc3)c(F)c2)C(=O)N1C]

and combinations thereof;
wherein R¹ and R² are each independently selected from the group consisting of hydrogen, CH₃, CH₂—OH, and CH(OH)—CH₃.

### Compositions

In another aspect, the present disclosure relates to a composition comprising a fluorogenic compound and/or a masked fluorogenic compound. In certain embodiments, the composition comprises a fluorogenic compound of Formula (I). In other embodiments, the composition comprises a masked fluorogenic compound of Formula (II) and/or Formula (IIa). In some embodiments, the composition further comprises a solvent and/or a carrier. The solvent and/or carrier can be any solvent and/or carrier known to a person of skill in the art. In certain embodiments, the solvent and/or carrier is organic. In other embodiments, the solvent and/or carrier is aqueous. In some embodiments, the solvent and/or carrier is a polar solvent such as DMSO or water. In certain embodiments, the compound of Formula (II) and/or Formula (IIa) is dissolved in minimal amount of DMSO (0.1-5% of final volume) and mixed with a near-neutral aqueous buffer (99.0-95% of final volume). In some embodiments, this mixture is passed through a filter and the collected filtrate can be used in in vitro, in cellulo, in vivo, or on chip applications.

Such a composition may be in a form suitable for administration to a subject (i.e. mammal), or the composition may further comprise one or more acceptable carriers, one or more additional ingredients, or some combination of these. The various components of the composition may be present in the form of a physiologically acceptable salt, such as in combination with a physiologically acceptable cation or anion, as is well known in the art.

Compositions that are useful in the methods of the invention may be suitably developed for inhalation, oral, rectal, vaginal, parenteral, topical to the skin, transdermal, pulmonary, intranasal, buccal, ophthalmic, intrathecal, intravenous, or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

Although the descriptions of compositions provided herein are principally directed to compositions suitable for ethical administration to humans, it is understood by the skilled artisan that such compositions are generally suitable for administration to animals of all sorts. Modification of compositions suitable for administration to humans in order to render the compositions suitable for administration to various animals is well understood, and the ordinarily skilled veterinary pharmacologist can design and perform such modification with merely ordinary, if any, experimentation.

The composition of the invention may comprise a preservative from about 0.005% to 2.0% by total weight of the composition. The preservative is used to prevent spoilage in the case of exposure to contaminants in the environment.

### Methods

### Method of Detecting a Disease Disorder in a Subject

In yet another aspect, the present disclosure relates to a method of detecting a disease or a disorder in a subject in need thereof. In certain embodiments, the method comprises expressing an RNA sequence comprising an RNA aptamer in the subject. In certain embodiments, the method comprises administering to the subject a compound of Formula (II). In certain embodiments, the method comprises detecting fluorescence emission associated with the aptamer. In some embodiment, fluorescence associated with the aptamer is derived from a fluorophore that is bound to the aptamer and is capable of fluorescing.

The RNA aptamer can have any sequence known to a person of skill in the art. In some embodiments, the RNA aptamer is a commercially available aptamer. In some embodiments, the RNA aptamer is Spinach, Baby Spinach, Corn, or Broccoli. In certain embodiments, the RNA aptamer is RNA_{Spinach}. In other embodiments, the RNA aptamer is RNA_{BabySpinach}. In other embodiments, the RNA aptamer comprises any functional RNA structure with the Spinach consensus sequence. In other embodiments, the consensus sequence comprises the binding site or catalytic core of the RNA_{Spinach}. In some embodiments, the RNA aptamer sequence is described in U.S. Pat. No. 9,664,676, the entire contents of which are incorporated herein by reference. The expression of the RNA aptamer in the subject can be performed using any method known to a person of skill in the art, including but not limited to the disclosure of U.S. Pat. No. 9,664,676.

The compound of Formula (II) can be administered to the subject using any method known to a person of skill in the art. The compound of Formula (II) can be any masked fluorogenic ligand of Formula (II) described elsewhere herein. In some embodiments, the compound of Formula (II) is a compound of Formula (IIa).

In some embodiments, administering to the subject a compound of Formula (II) further comprises deprotecting the compound of Formula (II) (i.e., removing the

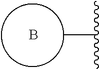

group completely or removing a portion of

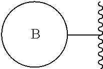

such that the remaining molecule acts as a fluorogenic ligand) via a reaction with a biomarker, producing a fluorogenic ligand. The biomarker can be any biomarker described elsewhere herein. In some embodiments, the biomarker is a biomarker that is associated with a particular disease or disorder or a particular class of diseases or disorders. In certain embodiments, the reaction between the biomarker and the compound of Formula (II) cleaves the bond between

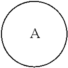

and E of Formula (II), producing a fluorogenic ligand of Formula (I).

In some embodiments, deprotecting the compound of Formula (II) via a reaction with a biomarker further comprises binding the fluorogenic ligand to the RNA aptamer. In certain embodiments, a fluorogenic ligand of Formula (I) binds to the RNA aptamer. In certain embodiments, the binding of the fluorogenic ligand to the RNA aptamer leads to an emission of fluorescence.

The fluorescence emission associated with the aptamer can be detected using any method known to a person of skill in the art. In some embodiments, there is no fluorescence emission associated with the aptamer, indicating the disease or disorder the subject is being screened for has not been detected and therefore the subject does not suffer from that disease or disorder. In other embodiments, there is fluorescence emission associated with the aptamer, indicating the disease or disorder the subject is being screened for has been detected and therefore the subject does suffer from that disease or disorder.

In other embodiments, the disease or disorder results in the production of hydrogen sulfide. In other embodiments, the disease or disorder results in the production of superoxide. In other embodiments, the disease or disorder results in the production of Cu(I). In other embodiments, the disease or disorder results in the production of Fe(II). In other embodiments, the disease or disorder results in the production of Zn(II). In certain embodiments, the disease or disorder results in the overproduction of hydrogen peroxide during abnormal intracellular redox homeostasis. In other embodiments, the disease or disorder results in the production of peroxynitrite. In other embodiments, the disease or disorder results in the overproduction of glutathione. In other embodiments. In other embodiments, the disease or disorder results in the overexpression of hepatic lipase. In other embodiments, the disease or disorder results from the overexpression of Cathepsin B. In other embodiments, the disease or disorder interferes with the activation of a caspase. In other embodiments, the disease or disorder leads to elevated levels of alkaline phosphatase. In some embodiments, the disease or disorder is selected from the group consisting of: cancer such as breast cancer, lung cancer, or ovarian cancer, an inflammatory disease or disorder, diabetes, a cardiovascular disease or disorder, a neurodegenerative disease or disorder such as amyotrophic lateral sclerosis (ALS) and traumatic brain injury, hepatic steatosis, Huntington's disease, Alzheimer's disease, dysregulated osteoblastic activity, stroke, and combinations thereof.

In some embodiments, the compound of Formula (II) and/or Formula (IIa) is administered to the subject after the subject has received a therapeutic treatment for a disease or disorder. In some embodiments, the intensity of the fluorescence emission can be correlated to the amount of biomarker associated with a disease or disorder present in the subject. In some embodiments, the intensity of the fluorescence emission before the therapeutic treatment can be compared to the intensity of the fluorescence emission after the treatment in order to monitor the efficacy of the treatment.

### Method of Detecting a Disease Disorder in a Biological Sample

In yet another aspect, the present disclosure relates to a method of detecting a disease or a disorder in a biological sample. In certain embodiments, the method comprises providing a chip comprising a grafted RNA aptamer. In certain embodiments, the method comprises contacting the chip with a biological sample. In certain embodiments, the method comprises contacting the biological sample with a compound of Formula (II). In certain embodiments, the method comprises rinsing the chip. In certain embodiments, the method comprises detecting fluorescence emission associated with the aptamer.

The grafted RNA aptamer can be any RNA aptamer known to a person of skill in the art. Exemplary RNA aptamers are described elsewhere herein. In certain embodiments, the chip comprising the grafted RNA aptamer is a titanium or silicon chip. In some embodiments, the titanium chip comprises titanium-based nanoneedles. In other embodiments, the silicon chip comprises silicon-based nanoneedles. In certain embodiments, the RNA aptamer is grafted to the chip via an amino group at the 5′- or 3′-end of the aptamer which conjugates with an oxidatively polymerized catecholamine on the surface of the chip. In some embodiments, the oxidatively polymerized catecholamine is a polymer of dihydroxyphenylalanine (DOPA) and/or dopamine (DA). In other embodiments, the RNA aptamer is grafted to the chip using click chemistry. In certain embodiments, the click chemistry comprises a reaction between an RNA aptamer modified with an azide and an oxidatively polymerized catecholamine on the surface of the chip wherein the catecholamine is modified to comprise an alkyne group. In some embodiments, the oxidatively polymerized catecholamine is a polymer of dihydroxyphenylalanine (DOPA) and/or dopamine (DA) wherein the DOPA and/or the DA is modified to comprise an alkyne.

The chip can be contacted with the biological sample using any technique known to a person of skill in the art. The biological sample may be prepared in any fashion necessary to allow a biomarker present in the biological sample to contact the surface of the chip. In certain embodiments, wherein the chip comprises titanium or silicon nanoneedles, contacting the chip with a biological sample comprises at least partially piercing the cell membrane of cells present in the biological sample. In certain embodiments, at least partially piercing the cell membrane releases a biomarker present in the cell. In some embodiments, the biological sample is obtained from a subject after the subject after the subject has received a therapeutic treatment for a disease or disorder. In other embodiments, the biological sample is obtained from a subject who is suspected to have a disease or disorder.

The biological sample can be contacted with a compound of Formula (II) using any technique known to a person of skill in the art. In some embodiments, the compound of Formula (II) is a compound of Formula (IIa). Exemplary compounds of Formula (II) and Formula (IIa) are described elsewhere herein.

In some embodiments, contacting the biological sample with a compound of Formula (II) further comprises deprotecting the compound of Formula (II) via a reaction with a biomarker present in the biological sample, producing a fluorogenic ligand. The biomarker can be any biomarker described elsewhere herein. In some embodiments, the biomarker is is associated with a particular disease or disorder or a particular class of diseases or disorders. In certain embodiments, the reaction between the biomarker and the compound of Formula (II) cleaves the bond between

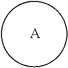

and E of Formula (II), producing a fluorogenic ligand of Formula (I).

In some embodiments, deprotecting the compound of Formula (II) via a reaction with a biomarker further comprises binding the fluorogenic ligand to the RNA aptamer. In certain embodiments, a fluorogenic ligand of Formula (I) binds to the RNA aptamer. In certain embodiments, the binding of the fluorogenic ligand to the RNA aptamer leads to an emission of fluorescence. In some embodiments, the intensity of the fluorescence emission can be correlated to the amount of biomarker associated with a disease or disorder present in the biological sample.

The fluorescence emission from the aptamer can be detected using any method known to a person of skill in the art. In some embodiments, there is no fluorescence emission associated with the aptamer, indicating the disease or disorder the biological sample is being screened for has not been detected and therefore the sample does not contain a biomarker associated with that disease or disorder. In other embodiments, there is fluorescence emission associated with the aptamer, indicating the disease or disorder the biological sample is being screened for has been detected and therefore the sample contains a biomarker associated with that disease or disorder. In some embodiments, the intensity of the fluorescence emission can be correlated to the amount of biomarker associated with a disease or disorder present in the biological sample. In some embodiments, the intensity of the fluorescence emission in a biological sample taken from a subject before the subject received a therapeutic treatment can be compared to the intensity of the fluorescence emission in a biological sample taken after the treatment in order to monitor the efficacy of the treatment.

In certain embodiments, the disease or disorder results in the overproduction of hydrogen peroxide during abnormal intracellular redox homeostasis. In other embodiments, the disease or disorder results in the production of hydrogen sulfide. In other embodiments, the disease or disorder results in the production of superoxide. In other embodiments, the disease or disorder results in the production of Cu(I). In other embodiments, the disease or disorder results in the production of Fe(II). In other embodiments, the disease or disorder results in the production of Zn(II). In other embodiments, the disease or disorder results in the production of peroxynitrite. In other embodiments, the disease or disorder results in the overproduction of glutathione. In other embodiments. In other embodiments, the disease or disorder results in the overexpression of hepatic lipase. In other embodiments, the disease or disorder results from the overexpression of Cathepsin B. In other embodiments, the disease or disorder interferes with the activation of a caspase. In other embodiments, the disease or disorder leads to elevated levels of alkaline phosphatase. In some embodiments, the disease or disorder is selected from the group consisting of: cancer such as breast cancer, lung cancer, or ovarian cancer, an inflammatory disease or disorder, diabetes, a cardiovascular disease or disorder, a neurodegenerative disease or disorder such as amyotrophic lateral sclerosis (ALS) and traumatic brain injury, hepatic steatosis, Huntington's disease, Alzheimer's disease, dysregulated osteoblastic activity, stroke, and combinations thereof.

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Small-Molecule Designs for Activity-Based Aptamer (ABA) Systems that Detect Disease Biomarkers

Biological implementation of functional nucleic acid sequences to detect an inorganic metabolite or an enzyme is not well known. The present disclosure addresses these challenges by providing an activity-based aptamer (ABA) system in which designer small molecules enable a single RNA aptamer sequence to detect multiple as well as structurally diverse cellular biomarkers, including organic and inorganic molecules, and enzymes. This technology has a great deal of translational potential, as these biomarkers can be associated to a critical human disease. The ABA technology is highly adaptable, as it can be tuned to detect a wide range of disorders, such as cancers with metastatic potential, cardiovascular and skeletal anomalies, hepatic steatosis, and neurodegenerative diseases.

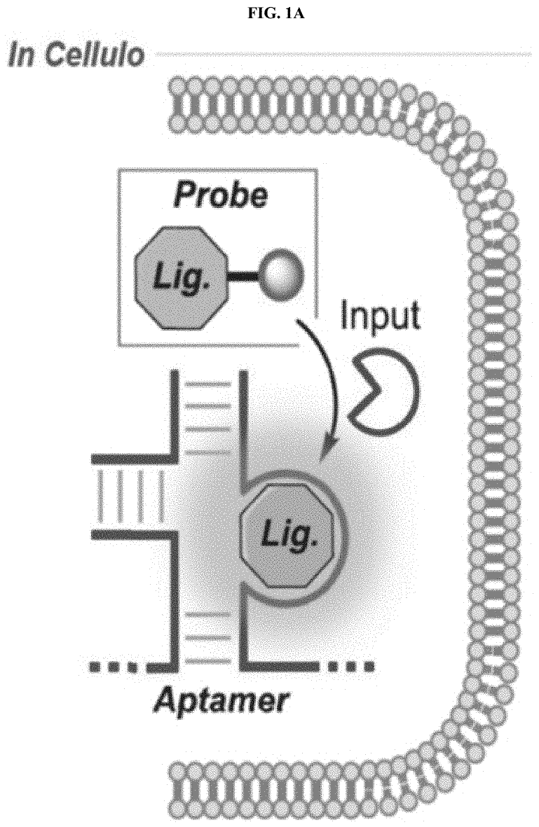

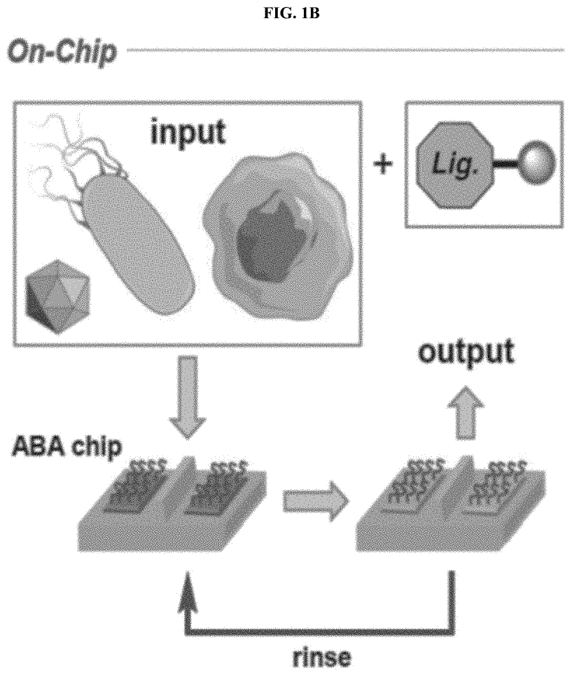

The disclosed ABA biosensors use small molecules that possess superior biophysical and biochemical properties in comparison to most fluorogenic probes. The ABA biosensors comprise a fluorogenic ligand that has been modified using a biomarker-specific chemical modification. These rationally designed structural modifications of the fluorogenic ligands can disrupt aptamer binding, thus significantly reducing their fluorescence emission. This chemical modification prevents aptamer-ligand binding interactions, until, in the presence of a biomarker, which is typically produced at elevated levels under disease conditions, this chemical modification is removed, providing the native ligand structure. The free native ligand then binds to the aptamer, resulting in a significant increase in its fluorescence quantum yield and a strong fluorescence signal (FIG. 1A). Free ABA ligands (i.e. unbound to the aptamer) will essentially not produce a background signal. Only their aptamer-bound states will undergo radiative dissipation under excitation. Consequently, the proposed technology will not be subject to erroneous results from nonspecific (bio)molecular interactions. The ABA probes and ligands are derived from Gly-Tyr-Ser. This is the fluorogenic core found in wild-type GFP, which is a bioorthogonal macromolecule. Therefore, these small molecules exhibit minimal cytotoxicity.

### Research Strategy

The engineered bioorthogonal and adaptable ABA systems emit light in response to a specific metabolite, opening a new platform for high-fidelity disease detection and interventions.

To this end, a diverse range of biomarkers are tested, including inorganic oxidants: hydrogen peroxide (H₂O₂) and peroxynitrite (ONOO⁻), an organic reductant: gluthathione (GSH), and enzymes: hepatic lipase (HL), cathepsin B, the caspase family, and alkaline phosphatase (ALP).

H₂O₂ is a reactive oxygen species (ROS) known to be overproduced during abnormal intracellular redox homeostasis, especially in cases of mitochondrial dysfunction and oncogene activity. Studies show that elevated cellular concentrations of H₂O₂ play a critical role in cancer progression and metastasis.

ONOO⁻ is a toxic reactive nitrogen species (RNS) whose production is associated with acute and chronic inflammatory processes, diabetes, cardiovascular disorders, and neurodegenerative disorders, such as amyotrophic lateral sclerosis (ALS) and traumatic brain injury.

GSH is a tripeptide-thiol that serves in cellular detoxification by acting as a ROS scavenger. GSH exists at abnormally high levels in breast, ovarian, and lung cancer cells. Excessive generation of GSH results from ROS-dependent overexpression of GSH-synthesizing enzymes. Cancer cells that maintain high intracellular GSH content develop drug resistance.

HL is a phospholipase that is overexpressed during fat accumulation in the liver due to hepatic steatosis, a critical liver dysfunction. Hepatic steatosis progresses into non-alcoholic fatty liver disease—the most prevalent chronic liver pathology in developed countries—and ends with liver cirrhosis. Effective diagnosis of hepatic steatosis is an unmet need in combating liver failure.

Cathepsin B is a cysteine protease whose overexpression promotes a malignant phenotype of breast cancer cells and contributes to progression of cancer cells into metastatic types. Studies show that breast cancer patients with a high content of cathepsin B in their primary tumors have an increased risk of relapse.

Caspases are a family of endoproteases involved in both intrinsic and extrinsic control of inflammation and apoptosis. Active forms of caspases are present in neurons prior to the development of neurodegenerative diseases including Huntington's Disease, ALS, and Alzheimer's. Apoptotic caspases, such as caspase-1 and caspase-3, have been found to contribute to neuronal death in the late stages of these diseases.

ALPs are glycoprotein enzymes that catalyze the hydrolysis of orthophosphate monoesters within a pH range of 7.5-9.5. They are commonly found on the plasma membranes of bone, liver, intestine, and kidney cells. Elevated levels of ALP in the blood is correlated with dysregulated osteoblastic activity and cardiovascular diseases, including stroke.

Development of aptamer systems as imaging tools enables high-fidelity detection of biomarkers, which illuminate aberrant metabolic pathways at the molecular level. Discoveries driven by the disclosed ABA systems lead to advanced molecular detection strategies, such as those that can provide early and reliable diagnosis of critical human diseases.

### Example 2: Construction of ABA Systems that Light Up in Response to a Disease-Relevant Biomarker

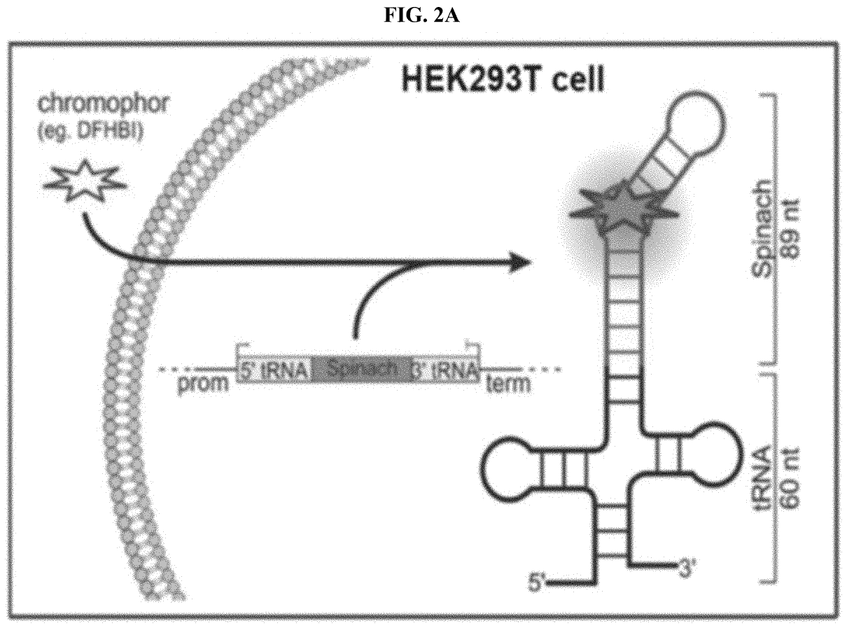

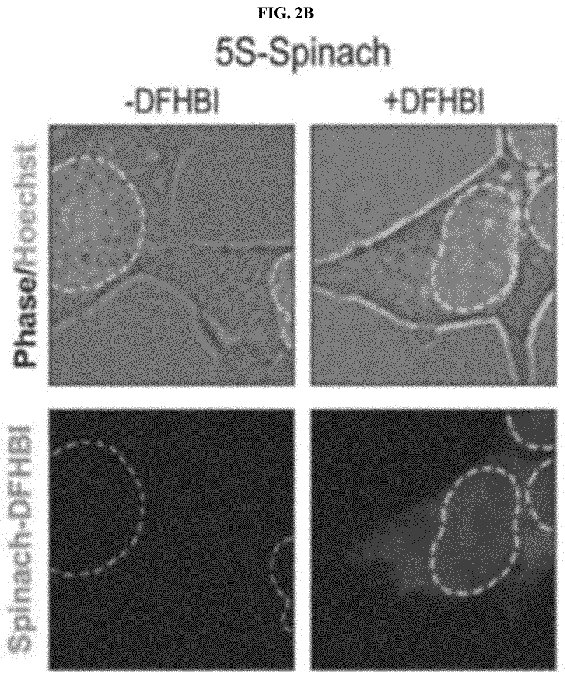

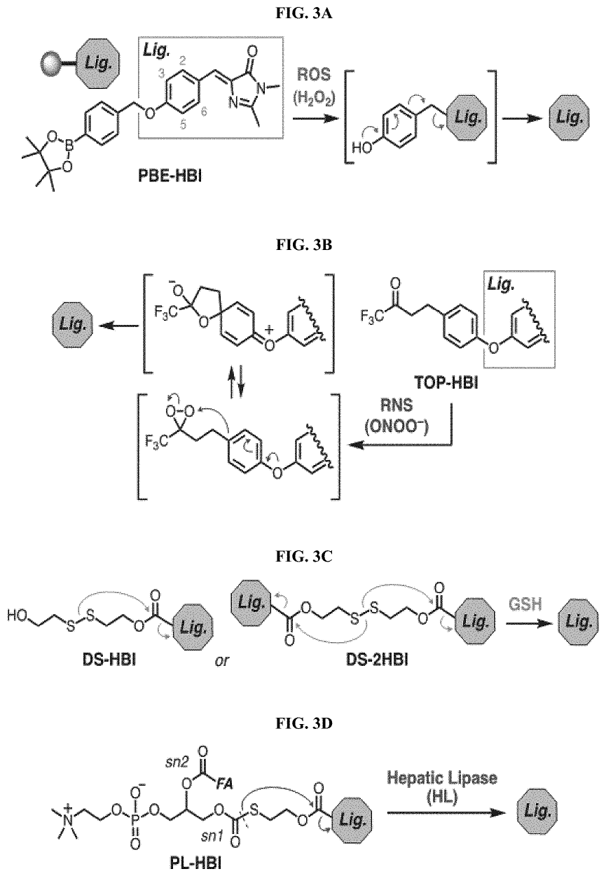

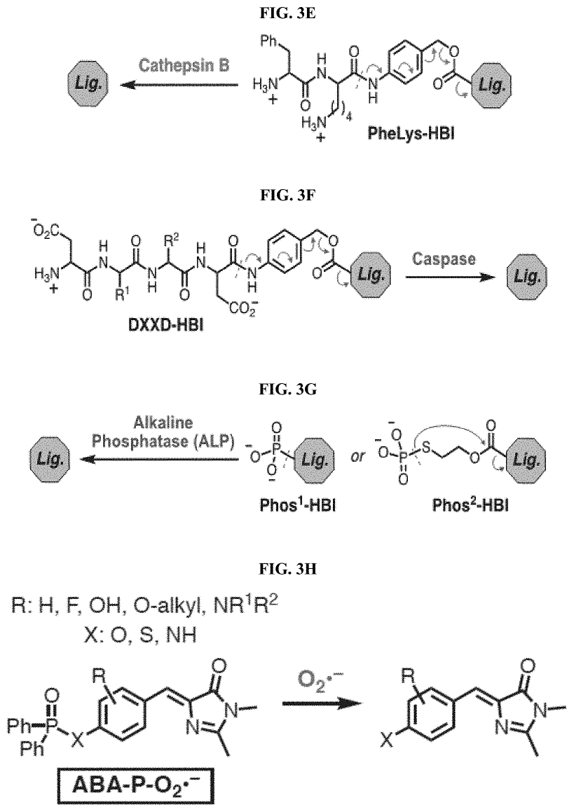

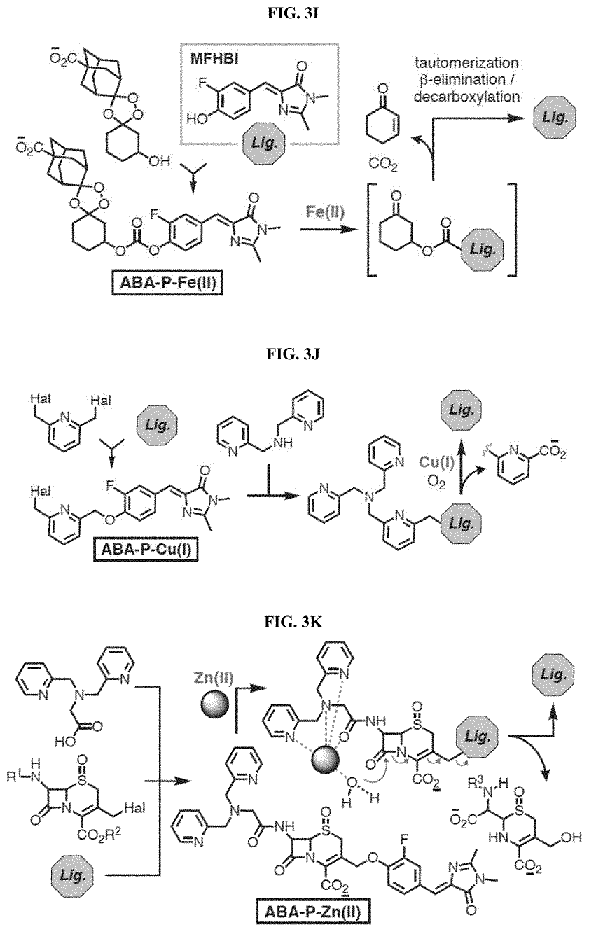

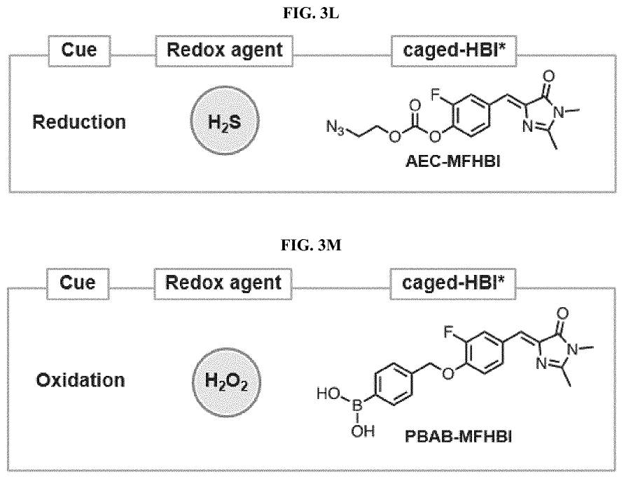

Aequorea green fluorescent protein (GFP) has a Φ_{f} of ˜0.8 at 504 nm; its fluorogenic core is composed of 4-hydroxy-benzylidene imidazolinone (HBI). The Φ_{f} values of free HBI and its derivatives are on the order of 10⁻⁴; however, they reach levels similar to that of GFP upon binding RNA aptamers (Paige, J. S. et al., Science, 2011, 333:642-646) (FIG. 2). The instant ABA technology is centered on masking the aptamer ligand, specifically the phenolic oxygen of HBI (FIGS. 3A-3M), with a group that sterically prevents aptamer binding. Reaction with an input (i.e., an inorganic or organic metabolite, or enzyme) unmasks the ligand, allowing it to bind the aptamer and drastically amplify its Φ_{f}.

In the ABA system described herein, 4-hydroxy-benzylidene imidazolinone (HBI)-based ligands can be used, which are caged at their phenolic oxygen with a moiety that prevents aptamer binding. Reaction between this moiety and its cognate biochemical input (e.g., biomarker) uncages the ligand, allowing it to bind the aptamer, which leads to a drastic increase in fluorescence.

Based on preliminary ligand designs, the monofluoro-HBI (MFHBI) framework proved to have an optimal reactivity profile, as compared to HBI and DFHBI. However, each of these ligand species permit aptamer binding and fluorescence. Thus, the present invention comprises the use of each of MFHBI, HBI, and DFHBI, inter alia, in conjunction with a suitable caging group. One skilled in the art would appreciate that the compounds described herein comprising a ligand (e.g, MFHBI, HBI, or DFHBI) conjugated to a particular caging group are not limited to those which are explicitly referenced and/or exemplified herein, and each combination of caging functionality and ligand is contemplated herein for use in the present invention, including methods of use of such compounds for detection of one or more redox agents (e.g., H₂O₂, ONO₂⁻, and O₂⁻) and/or transition metals (e.g., Fe(II), Cu(II), and/or Zn(II)).

### Construction and In Vitro Validation of the Small Molecule Library for ABA Systems

H₂O₂ has been shown to selectively convert p-tolylboronates into phenolic intermediates that undergo rapid eliminations (Carroll, V. et al., J. Am. Chem. Soc., 2014, 136:14742-14745; Chung, C. Y. S. et al., J. Am. Chem. Soc., 2018:140, 6109-6121). Thus, for ABA-mediated fluorescence detection of H₂O₂, a phenylboronic ester of HBI was designed, PBE-HBI (FIG. 3A), which generates free HBI through a similar oxidative elimination mechanism (arrows).

The RNS-detecting ABA system uses TOP-HBI (FIG. 3B). ONOO⁻ oxidizes the ketone in TOP-HBI to a dioxirane intermediate, which cyclizes to a spirocyclic hemiacetal (purple arrows) and subsequently hydrolyzes to HBI. Previous studies have shown that p-(4,4,4-trifluoro-3-oxobutyl)phenyl moiety undergoes rapid (˜15 min) oxidative cleavage with 100-300 μM of ONOO⁻ (Reed, J. W. et al., J. Am. Chem. Soc., 1974, 96:1248-1249). For in vitro testing, ONOO⁻ can be chemically synthesized using established protocols. To validate the fidelity of this design, RNS fluorescence assays can be performed in the presence of ROS (e.g., .OH, O₂.⁻, H₂O₂).

For GSH detection, disulfide-substituted carbonates of HBI are synthesized, DS-HBI and DS-2HBI (FIG. 3C). These molecules undergo a disulfide-thiol exchange with GSH, forming a free thiol product. Thiols are potent nucleophiles in physiological conditions. Therefore, this thiol product can attack the carbonate carbon center (gold arrows), which is located at a molecular distance where cyclization is favored (Gilmore, K. et al., WIREs Comput. Mol. Sci., 2016, 6:487-514). In vitro, 1-5 mM GSH has been reported to cleave 40-70% of disulfide-substituted carbonates within 5 min (Maiti, S. et al., J. Am. Chem. Soc., 2013, 135:4567-4572). For these assays, an aqueous solution of GSH can be externally introduced.

HL is a phospholipase Ai family enzyme known to catalyze the lipolysis of phospholipids, including those with structural alterations (Darrow, A. L. et al., J. Lipid Res., 2011, 52:374-382), at the sn1 acyl ester bond (Miksztowicz, V. et al., Arterioscler. Thromb. Vasc. Biol., 2012, 32:3033-3040). HL can cleave the sn1 thiocarbonate in PL-HBI (FIG. 3D), triggering a decarboxylative release of free HBI (arrows). HL-catalyzed lipid cleavage can have Kₘ values ranging from 0.1 to 2.5 mM (Shirai, K. et al., Biochim. Biophys. Acta, 1984, 795:1), which correlates with concentrations of PL-HBI for a reliable detection. Because HL is not readily available, recombinantly expressed HL can be used.

Cathepsin B targets amide bonds after PheLys dipeptide (Miller, K. et al., Angew. Chem. Int. Ed., 2009, 48:2949-2954). Cathepsin B can remove the dipeptide in PheLys-HBI (FIG. 3E), forming an aminobenzene, which undergoes a decarboxylative elimination of HBI (magenta arrows). Cathepsin B catalysis can have a Kₘ value of ˜1.4 nM, which correlates with concentrations of PheLys-HBI for a reliable detection. Here, commercial human cathepsin B can be used.

Caspases cleave peptide bonds after specific Asp (D) residues, commonly in DXXD-peptide (X=Gly, Ala, Ser, or Thr). DXXD-HBI (FIG. 3F) is synthesized to generate HBI via a similar decarboxylative elimination mechanism (green arrows). Caspases cleave DXXD substrates with Kₘ values ranging from 0.3 to 5.5 μM (Faustin, B. et al., Mol. Cell, 2007, 25:713-724; Boeneman, K. et al., J. Am. Chem. Soc., 2009, 131:3828-3829), which correlates with concentrations of DXXD-HBI for a reliable detection. Here commercial human caspases that are heavily linked to neural disorders (Chen, M. et al., Nat. Med., 2000, 6:797-801) can be used (e.g., active caspase 1 and 3).

Detection of ALP occurs by ALP-catalyzed dephosphorylation of Phos¹-HBI or Phos²-HBI (FIG. 3G). ALP catalysis can have a Kₘ value of 0.1-0.7 mM, which correlates with concentrations of Phos-HBIs for a reliable detection. Here, previously reported human ALPs, including liver/bone/kidney (L/B/K) ALP (Hoylaerts, M. F. et al., J. Biol. Chem., 1997, 272:22781-22787), can be used.

Regarding transition metal-mediated uncaging, known heteroatom-metal reactivities are leveraged in the development of MFHBI caging moieties, including: (a) decarboxylative elimination of β-carbonate or β-carbamate substituted spirocyclic endoperoxide upon reaction with Fe(II); (b) oxidative removal of tris[(2-pyridylmethyl)amino]-alkyls by reaction with Cu(I); and (c) hydrolysis of β-lactam thianones with Zn(II) (FIGS. 3I-3K).

In view of these metal-organic functional group reactivity profiles, the MFHBI-caging moieties are prepared from: (a) an adamantanone starting material (i.e., for preparation of the spirocyclic endoperoxide); (b) commercially available bis-halo pyridine and/or 2,2′-dipicolylamine (i.e., for preparation of the tris[(2-pyridylmethyl)amino) alkyls); commercially available dipicolylamino acetic acid and β-lactam halide (i.e., for preparation of β-lactam thianones).

Regarding detection of redox reagents (e.g., O₂⁻ and H₂S), a phosphinate caging group can be utilized for detection of superoxide, resulting in release of the phenoxy moiety in MFHBI upon exposure to O₂⁻ (FIG. 3H), whereas a carbonate-linked alkyl azide moiety can be used for detection of H₂S, resulting in release of the phenoxy moiety in MFHBI upon exposure to H₂S. (FIG. 3L).

Direct quantitative measurement of the cellular concentrations of these biomarkers in living organisms is challenging, which further emphasizes the importance of the disclosed invention. Relevant information regarding the concentrations and how these values have been measured is summarized in Table 1. The formation of free HBI and/or MFHBI is monitored by monitoring the change in intensity of HBI/MFHBI-specific fluorescence. Additionally, NMR and mass spectroscopic investigations are performed to validate the transformations proposed in FIGS. 3A-3M. RNA_{BabySpinach}(51-nt) (Warner, K. D. et al., Nat. Struct. Mol. Biol., 2014, 21:658-63.) can be used as the aptamer sequence in the ABA system, which can be obtained via induced biosynthesis or solid-phase oligonucleotide synthesis.

TABLE 1
Measurement of cellular concentration of relevant
biomarkers by alternative methods

| Biomarker | Normal | | Disease | | Measurement |
|---|---|---|---|---|---|
| H₂O₂ | 0.1-1 | μM | 10-100 | μM | Protein activity assay in human |
| | | | | | alveolar adenocarcinomic cells |
| ONOO⁻ | 6-30 | μM | 50-100 | μM | 3-Nitrotyrosine production (min⁻¹) in |
| | | | | | mice reticulum sarcoma cells |
| GSH | 1-2 | mM | 10 | mM | Fluorescence from small molecule in |
| | | | | | human alveolar adenocarcinomic cells |
| HL | 13-21 μEq | | 680-995 μEq | | Free fatty acid (FFA) formation in |
| | (FFA/mL/h) | | (FFA/mL/h) | | homozygous (+/+) human HL |
| | | | | | transgenic mice |
| Cathepsin B | 10-533 | | 217-2310 | | ELISA assay in human breast |
| | ng/mg protein | | ng/mg protein | | tissues |
| Caspase-3 | 1676-3666 | | 6290-12503 | | Fluorescence from small molecule |
| | arbitrary FU | | arbitrary FU | | in cell lysates of colon carcinoma |
| ALP | 36-130 | IU/L | >130 | IU/L | UV absorbance of small molecule |
| | | | | | used in human blood sample |

### Results

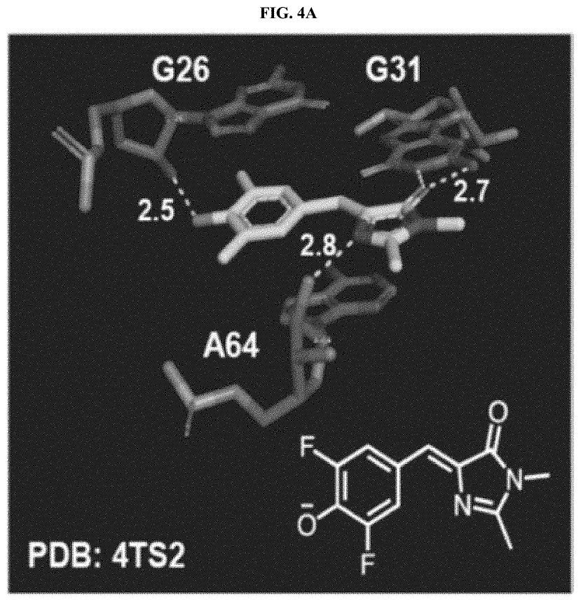

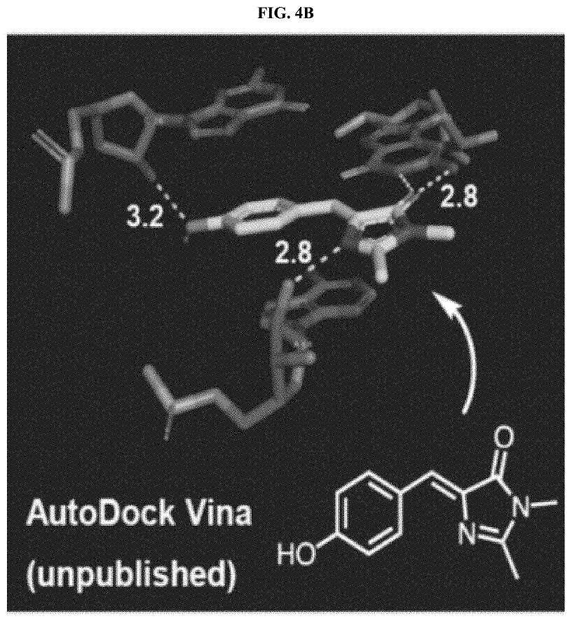

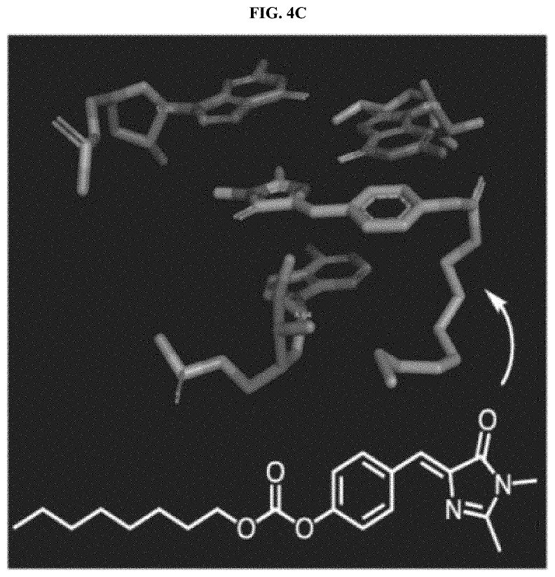

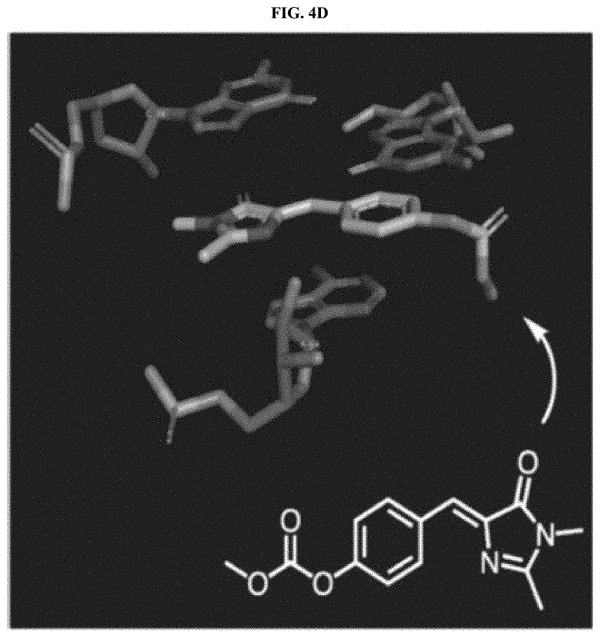

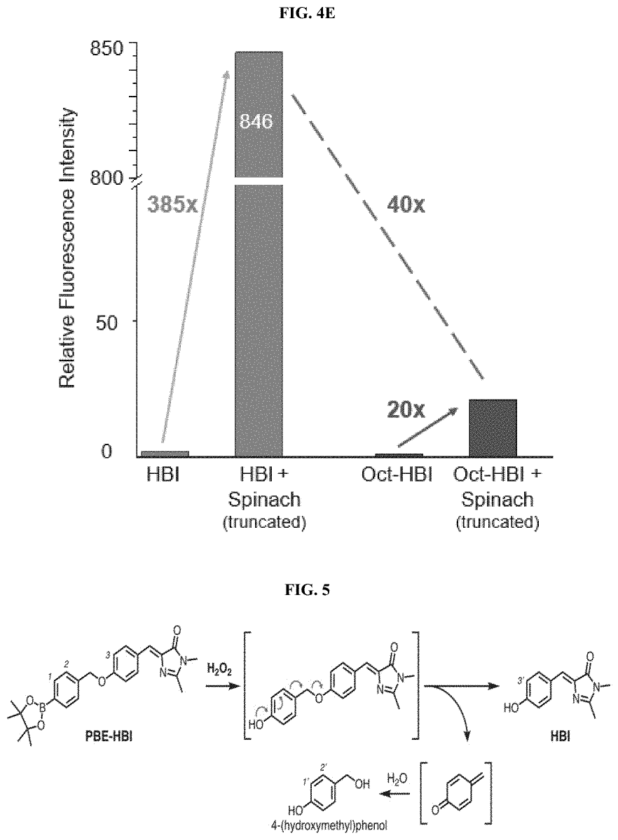

Ligand docking is a useful tool to screen virtual library of molecules to predict binding affinity and conformation of a ligand within a binding pocket. To gain a theoretical insight into the effect of chemical modifications of HBI on its binding interactions with RNA, a robust docking method named Auto-Dock Vina was used (FIGS. 4A-4D). First, the validity of this method was tested by comparing the proposed HBI-RNA_{Spinach} docking (FIG. 4B) with the X-ray crystal structure of the native ligand-RNA_{Spinach} [FIG. 4A; the co-crystallized ligand in the reported study is 3,5-difluoro-HBI (DFHBI)]. To attain the initial HBI structure for docking, optimizations were performed in gas phase via density functional theory calculations using B3LYP, 6-31G basis set in Gaussian16 software. The distances for key H-bonding interactions of docked HBI with G/G/A nucleobases of RNA_{Spinach} (FIG. 4B) were similar to those measured for the crystal structure (FIG. 4A). Next, the phenolic oxygen of HBI was replaced with alkyl carbonate groups. Docking results revealed that the benzylidene conformation of these molecules changed from endo (FIGS. 4A and 4B) to exo (FIGS. 4C and 4D; octyl carbonate-modified HBI (Oct-HBI) and methyl carbonate-modified HBI, respectively) while all the key H-bonding ligand interactions within the G/G/A binding pocket were eliminated. The experimental binding assay with RNA_{BabySpinach}, (51-nt) (Warner, K. D. et al., Nat. Struct. Mol. Biol., 2014, 21:658-63), a truncated version of RNA_{Spinach}, and Oct-HBI (FIG. 4E) showed nearly 40 times decrease in fluorescence intensity compared to HBI-RNA_{BabySpinach} (21 vs. 846 RFU). These computational and experimental results suggested that a chemical modification that both masks the HBI phenolic oxygen and provides steric bulk prevent the binding of the fluorophore, thereby suppressing its fluorescence emission.

Encouraged by these results, the synthesis and in vitro validation of the H₂O₂-detecting system was the next focus. H₂O₂ has been reported to selectively convert p-tolylboronate groups into phenolic intermediates that undergo rapid elimination reactions. Thus, for ABA-mediated fluorescence detection of H₂O₂, a phenylboronic ester of HBI, PBE-HBI (FIG. 5) was designed which generates free HBI through an oxidative elimination mechanism (arrows). PBE-HBI was synthesized from HBI and 4-bromomethylphenyl-boronic acid pinacol ester under optimized nucleophilic displacement conditions.

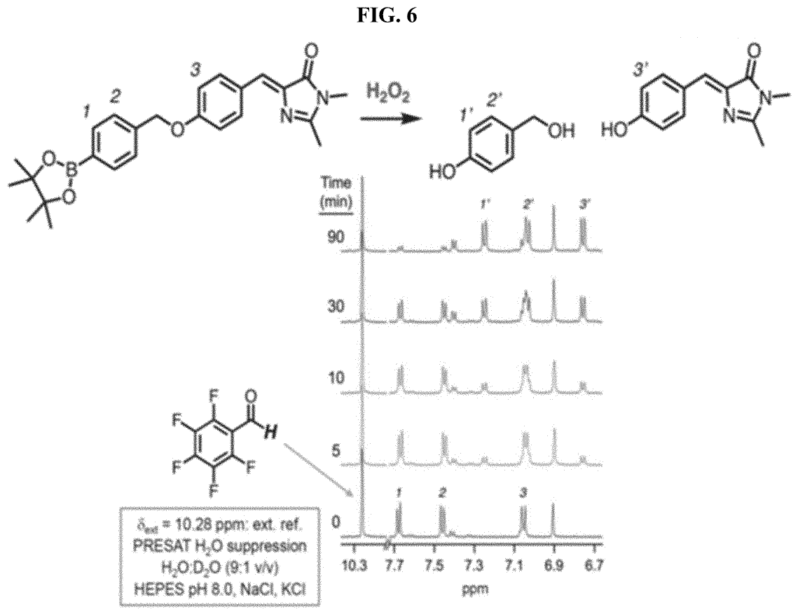

To test the chemical reactivity of PBE-HBI towards H₂O₂ at room temperature and characterize its oxidation products, ¹H-NMR studies were carried out where the oxidation progress was monitored over 90 min (FIG. 6). Results supported the proposed transformation of PBE-HBI to HBI (FIG. 5), where the depletion of the boronic ester (proton signals 1, 2, and 3) and the subsequent formation of HBI (proton signal 3′) as well as 4-(hydroxymethyl)phenol (proton signals 1′ and 2′) were observed.

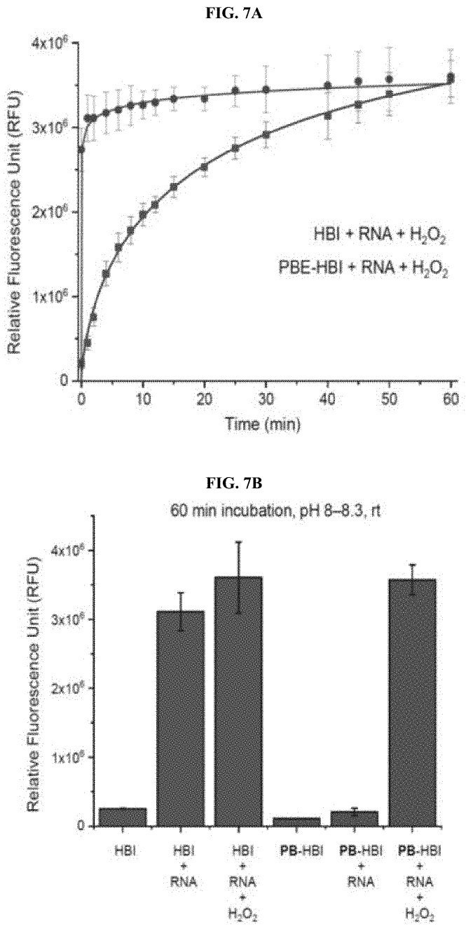

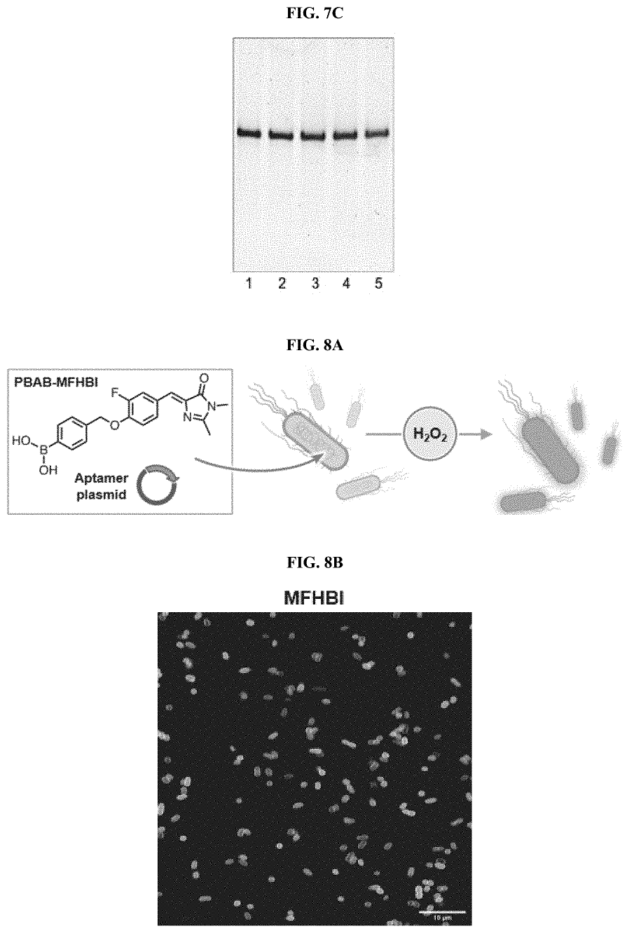

Next, the change in fluorescence for H₂O₂-treated aptamer solutions that contain HBI (positive control) and PBE-HBI (FIGS. 7A and 7B) was monitored. The RFU of PBE-HBI+RNA_{BabySpinach} was lower than that of HBI+RNA_{BabySpinach} by 14-fold at 0 min (no H₂O₂ present), validating that masking the phenolic oxygen of HBI inhibits RNA binding. Upon addition of H₂O₂ (100 μM), RFU of PBE-HBI+RNA_{BabySpinach} reached up to 92% of RFU of the control in 60 min. The insignificant, yet steady, increase of the fluorescence intensity for HBI+RNA_{BabySpinach} and PBE-HBI+RNA_{BabySpinach} mixtures (FIG. 7B) suggests that there is a prolonged ligand saturation period of the aptamer. Upon H₂O₂ addition, the solution pH increased from 8.0 to 8.4 while ˜10% growth in fluorescence intensity was measured for the HBI-containing samples. This change is attributed to the pH-dependent increase in the phenolate form of HBI (see FIG. 5), which is known to have a higher extinction coefficient than its phenol form. Notably, the fluorescence intensity of PBE-HBI+RNA_{BabySpinach}+H₂O₂ raised substantially and rapidly (˜4-fold at 5 min and ˜8-fold after 60 min, 2nd vs. 3rd blue bars), suggesting that the RNA technology works effectively in vitro. To investigate the backbone stability of RNA_{BabySpinach} under these oxidative conditions, PAGE analyses of RNA_{BabySpinach} solutions containing HBI (FIG. 7C, lanes 2 and 3) and PBE-HBI (FIG. 7C, lanes 4 and 5) were carried out. The gel analysis indicated that incubation of RNA_{BabySpinach} with H₂O₂ (up to 1 mM) for 60 min (FIG. 7C, lanes 3 and 5) does not lead to an observable RNA degradation such as phosphodiester backbone degradation.

Remarkably, the kinetics of H₂O₂ detection, which involves conversion of PBE-HBI to HBI, followed by HBI-aptamer binding, is fast. The incubation time to achieve 1/2 RFUₘₐₓ through this process is 6.8 (±0.5) min. Reliable detection of H₂O₂ is feasible in ˜1 min as the fluorescence intensity doubles. These findings are encouraging and serve as a standard for comparing the detection kinetics for other probes. The feasibility of detecting biomarkers with significant concentration discrepancies (3-20 fold) between normal and disease conditions (Table 1) should be comparable to that of H₂O₂. In certain embodiments, reliable detection of elevated ALP may require longer incubations with the respective ABA system (FIG. 3G), as ALP has a relatively small margin of variation. The rates of biomarker detection can be modulated by substituting the HBI carbon framework of the probes (FIGS. 3A-3G) with electron donating (—OMe, —NMe₂) or withdrawing (—F, —CF₃) groups.

### Example 3: Implementation of ABA Systems in Living Cells

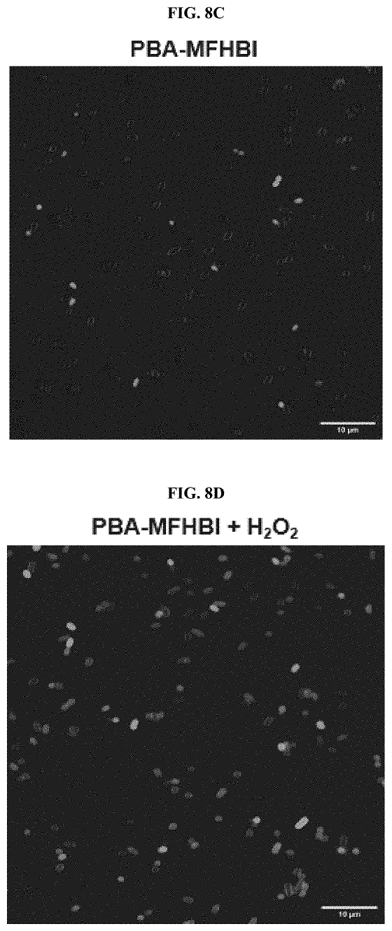

For histochemical detection of oxidative stress in living cells, the focus is first on E. coli competent cells (e.g., BL21-DE3) (FIGS. 8A-8D). For the imaging of H₂O₂, BL21-DE3 cells are transformed with plasmid DNA that contains a T7 promoter and RNA_{BabySpinach} or a custom triple baby spinach construct, which allows for the expression of three baby spinach RNA aptamers per transcription cycle. These cells are inoculated with isopropyl β-D-1-thiogalactopyranoside (IPTG) to induce RNA transcription. Subsequent immobilization of the cells on glass dish coated with poly-L-lysine (PLL) and incubation with an imaging buffer (IB) containing PBE-HBI or PBAB-MFHBI with H₂O₂ (100 μM) (FIG. 8D) shows a rapid increase in fluorescence within one hour, allowing for real time, fluorescence imaging of oxidative stress in cellulo. In the control sample (FIG. 8C), where exogenous H₂O₂ is absent, there is a very small increase in fluorescence. These results indicate that ABA technology can be utilized for live cell imaging to detect oxidative stress. HBI is known to be cell permeable, therefore, once formed, it binds to the intracellularly transcribed RNA aptamer. The results can guide additional studies with more complex and biologically relevant cell lines, in particular, any primary or established mammalian cells (e.g., HEK293, HeLa, and HUVEC, inter alia). Detection of endogenous H₂O₂ generation can also be studied by using molecular stimulants known to trigger mitochondrial ROS production, such as Phorbol 12-myristate 13-acetate (PMA).

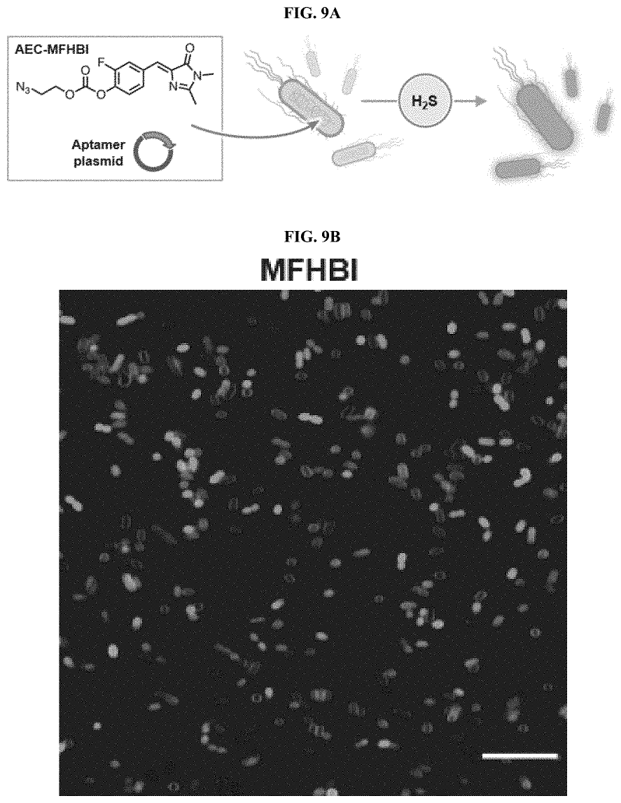

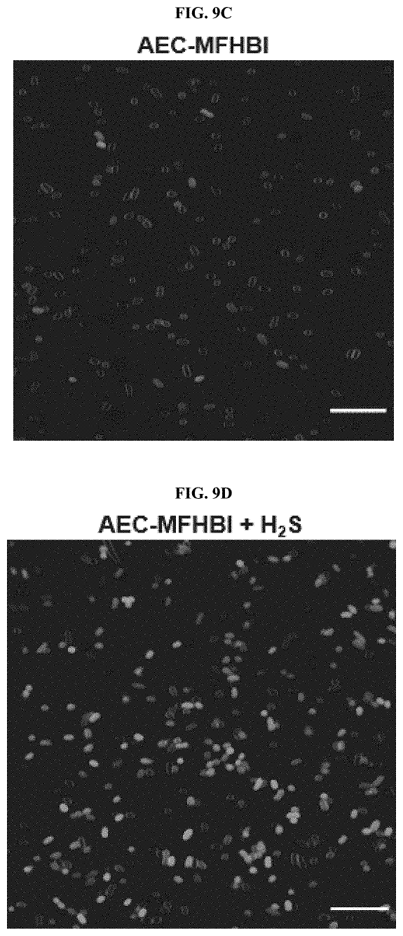

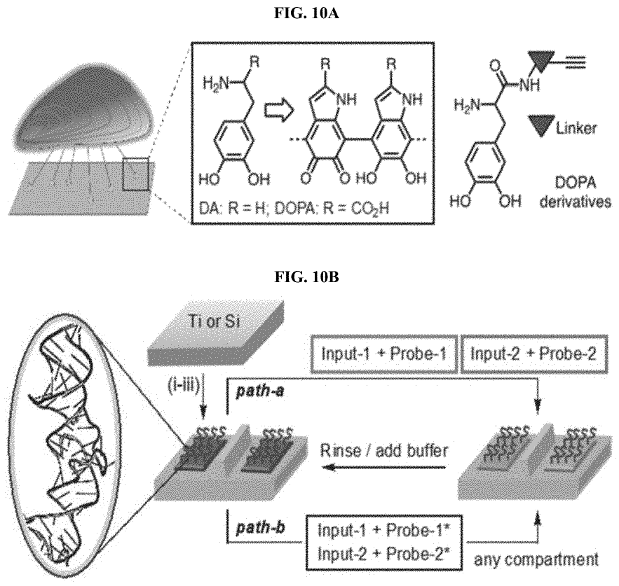

Imaging of hydrogen sulfide (H₂S) has been performed in an analogous manner to that which is described for the detection of H₂O₂. Specifically, BL21-DE3 cells are transformed with plasmid DNA that contains a T7 promoter and RNA_{BabySpinach} or a custom triple baby spinach construct, which allows for the expression of three baby spinach RNA aptamers per transcription cycle. These cells are inoculated with isopropyl β-D-1-thiogalactopyranoside (IPTG) to induce RNA transcription. Subsequent immobilization of the cells on glass dish coated with poly-L-lysine (PLL) and incubation with an imaging buffer (IB) containing the imaging agents and/or controls (i.e., MFHBI, FIG. 9B; or AEC-MFHBI, FIGS. 9C-9D), allowed real time fluorescence imaging of SH₂ in cellulo. In particular, upon introduction of H₂S (1 mM) (FIG. 9D), a rapid increase in fluorescence is observed within one hour unlike the control sample (FIG. 9C), wherein H₂S is absent. This data provides support for the assertion that AEC-MFHBI can be used to detect H₂S in cellulo.

For ABA-mediated, in-cellulo detection of metabolically produced nitrosative stress biomarker ONOO⁻, HUVEC is studied. First, the RNA_{BabySpinach} sequence is introduced into the cells through transfection. These cells are treated with an IB containing TOP-HBI (FIG. 3B) and the ONOO⁻ donor SIN-1.HCl. The specificity of the detection against ROS and other RNS is investigated by using corresponding ROS donors (e.g., xanthine/xanthine oxidase) and the NO donor (e.g., S-nitroso-N-acetyl-penicillamine).

To study GSH detection in living cells, model human breast adenocarcinoma cells known to self-produce GSH¹⁷ are used (e.g., MDA-MB-231). Here, an IB that contains DS-HBI or DS-2HBI (FIG. 3C) is proposed in a non-limiting manner, wherein the latter probe may facilitate a higher level of fluorescence emission, as it possesses two potential HBI molecules available for GSH-mediated elimination.

For detection of the enzymatic biomarkers, HL, cathepsin B, and caspases, HEK293 cells are studied as a proof-of-concept. Here, the aptamer gene is fused with the enzyme gene sequence, which is co-transcribed as an aptamer/enzyme mRNA. For HL expression, viral transfection is used. HEK293 cells are grown on a PLL coated glass surface after transfection with the aptamer/enzyme fused gene. Next, HEK293 cells are treated with an IB that contains a designer small-molecule [PL-HBI for HL (FIG. 3D), PheLys-HBI for cathepsin B (FIG. 3E), and DXXD-HBI for caspases (FIG. 3F)]. MC3T3-E1 cells are studied for ALP detection via Phos-HBIs (FIG. 3G). Intracellular ALP activity is stimulated with dexamethasone.

The feasibility of enzyme expressions and the proposed biochemical pathways that generate HBI through fluorescence imaging are evaluated. Results are compared to those obtained from positive controls, in which cells are transfected with the aptamer itself in a media that contains native HBI, and from negative controls, in which cell media lacks masked HBI.

Without wishing to be limited by any theory, although HBI is membrane permeable, the masked HBIs with anionic character (DXXD-HBI, Phos¹-HBI, or Phos²-HBI) may have limitations. To facilitate in cellulo accessibility of these probes, chemical modifications of the masking groups with amphiphilic moieties (e.g., fatty acyl chain or cell-penetrating short peptides) are explored that enhance probe-membrane interactions and increase cell localization without compromising the ability to react with biomarkers. Alternatively, electropermeabilization protocols reported for insertion of macromolecules into human cells can be employed (Celis, J. E. et al., J. Electroanal. Chem., 1990, 298:65-80). Alternatively, the enzyme of interest can be provided exogenously via plasmid co-transfection.

### Example 4: Development of Aptamer-Grafted Chips that Detect Biological Specimens

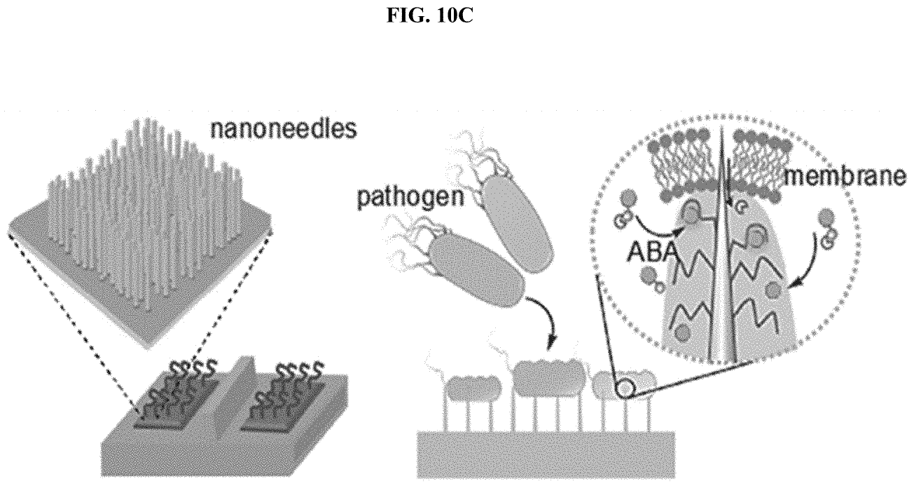

The generation of aptamer-grafted chips (FIG. 1B and FIGS. 10A-10C) is inspired by the remarkably strong adhesion of mussel foot protein Mefp-5 to virtually any kind of solid object. The key adhesive component in Mefp-5 is 3,4-dihydroxyphenylalanine (DOPA), a structural analog of dopamine (DA) (FIG. 10A). These catecholamines are capable of spontaneously coating solid surfaces via oxidative polymerization at pH 7-9 (Mrówczyński, R. et al., Polym. Int., 2016, 65:1288-1299). The resulting surfaces are chemically adaptable, as polymerized-DOPA/DA can conjugate with nucleophilic amines (Liu, Y. L. et al., Chem. Rev., 2014, 114:5057-5115). To this end, Ti and Si chips are grafted with aptamers that bear an amino group at either the 5′- or 3′-end. These aptamers are obtained by either solid-phase synthesis or enzymatic amino-nucleotide incorporation (e.g., using archaeal polymerases, such as therminator). As a second grafting approach, click chemistry is used, a robust cycloaddition reaction between an alkyne and azide group. This chemistry has been shown to be versatile for surface engineering and can allow for higher aptamer conjugation efficiencies. Recently, a drop coating method was developed that combines catecholamine polymerization with click chemistry to graft surfaces with bioactive molecules, such as cyclic RGDfK, BSA, and PEG. Therefore, effective aptamer grafting can be achieved using azide-containing RNAs and alkyne-containing DOPA derivatives (FIG. 10A). The azido-RNAs are obtained by solid-phase oligonucleotide synthesis, enzymatic incorporation, or from commercial sources. This approach enables site-selective aptamer immobilization, which is critical for the development of multipurpose biosensors.

This chip design can be expanded to engineer reusable and multiplexed sensors, where the ABA systems are able to continuously assay multiple inputs (FIG. 10B). Sustainable surface restoration is important for the reusability of aptamer chips. Studies concerning this are scarce, and most prior effort for reusing microarrayed nucleic acids has been devoted to stripping mechanisms through chemical denaturation. The current practice for stripping nucleic acid microarrays typically works at high temperatures (≥60° C.) or under chemical conditions that can be detrimental to aptamers. The chip design described herein serves as a more compatible alternative for preserving the activity of immobilized nucleic acids. The ABA systems work at a mild pH (˜8) and in the presence of divalent metal ions (e.g., Mg²⁺). Therefore, simply rinsing the chip surface at room temperature with nuclease-free water and reloading the chip with metal ions should restore its function.

The proposed technology enables a single chip, grafted with only one type of aptamer, to identify and differentiate multiple biomarkers. Multiplexed detection are conducted through various methods. In certain embodiments, photolithography is used to compartmentalize the chip surface with physical barriers to prevent diffusion of probes or ligands on the chip. This design principle allows for sequential detection of target biomarkers using respective probes at designated compartments of the chip (FIG. 10B, path-a). In other embodiments, different substituents are incorporated at the C2, C3, C5, and/or C6 positions of the probe's benzylidene ring (FIG. 10A). These structural variants produce different aptamer ligands, each with different benzylidene substitution. Substituents that are small in size can be chosen to avoid altering aptamer binding kinetics, while still inducing changes in the λₘₐₓ emission. Therefore, multiple, unique biomarkers present in a single sample can convert probes into free ligands, each of which will emit a distinguishable wavelength of light upon binding to the aptamer (FIG. 10B, path-b).

### Investigation of the Viability of the Aptamer Chips for Biological Specimens

High-aspect-ratio solid nano-scale structures, such as nanoneedles, have emerged as promising analytic probes (Chiappini, C., ACS Sensors, 2017, 2:1086-1102). These materials can penetrate cells and deliver molecular probes while maintaining a long half-life (Chiappini, C., ACS Sensors, 2017, 2:1086-1102; Xu, A. M. et al., Nat. Commun., 2014, 5:3613). Accordingly, surfaces can be engineered that are co-arrayed with aptamers and nanoneedles (FIG. 10C) to develop a platform for capture-assisted sensing of pathogenic specimens (bacterial or tumor cells). The Ti-based nanoneedle arrays can be produced on Ti foils via seed-assisted hydrothermal synthesis using TiCl₄ as seeding precursor. For the creation of Si-based nanoneedles, metal-assisted chemical (Ag⁺/HTF) etching can be applied to Si wafers based on established protocols. These needles pierce membranes to liberate biomarkers present in the specimen (FIG. 10C, right). With the infusion of the masked HBIs on the chip, biomarkers can be detected via fluorescence upon HBI-aptamer binding.

For the initial feasibility studies, E. coli and MDA-MB-231 cells can be used. In certain embodiments, one can design metabolite-targeting probes to detect highly infectious bacteria, such as Staphylococcus aureus or Pseudomonas aeruginosa.

The disclosed ABA-chips rely on RNAs, which are prone to nucleolytic degradation. Therefore, if the aptamers described herein prove to have inconveniently short half-lives when exposed to clinical samples, various strategies will be pursued. In certain embodiments, samples can be processed with controlled heating to inactivate nucleases. Ribonucleases found in bodily fluids can be inactivated through heating, which has been successfully used for CRISPR-Cas biosensing platforms. In other embodiments, chemically-modified oligonucleotides that are poor substrates for nucleases, and thus can evade premature degradation, can be used. These include oligonucleotides that possess biophysical properties similar to those of RNA, in particular, 2′-OMe-RNA, locked nucleic acid, threose nucleic acid, phosphorothioate RNA, and N3′→P5′ phosphoramidate DNA. To preserve the binding characteristics of the aptamer, these backbone modifications are incorporated primarily to the aptamer stem regions apart from the consensus sequences and motifs.

In conclusion, an innovative strategy has been presented that increases the relevance of aptamers in bioanalytical fields by introducing a method in which aptamers can detect inorganic compounds and enzymes, as opposed to a limited scope of structurally similar organic molecules. Furthermore, besides increasing the structural landscape of potential targets, the disclosed ABA technology allows a single aptamer to detect multiple analytes. In the context of aptamers, this virtually eliminates the requirement to find a new functional RNA sequence for each analyte. This study opens a paradigm of reactivity-guided engineering of small-molecule-RNA systems, which allows real-time trafficking of molecules that are present within living organisms to investigate their biological roles and behaviors.

### Enumerated Embodiments

The following exemplary embodiments are provided, the numbering of which is not to be construed as designating levels of importance:

Embodiment 1 provides a compound of Formula (II):

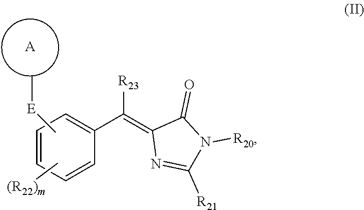

wherein:

R₂₀, R₂₁, and R₂₃ are each independently selected from the group consisting of hydrogen, deuterium, and optionally substituted C₁-C₆ alkyl;

each occurrence of R₂₂ is independently selected from the group consisting of deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;

each occurrence of R′ is independently selected from the group consisting of hydrogen, deuterium, hydroxy, C₁-C₆ alkyl, and C₁-C₆ alkoxy;

m is 0, 1, 2, 3, or 4;

E is selected from the group consisting of —O—, —S—, and —NH—;

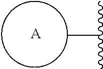

is selected from the group consisting of

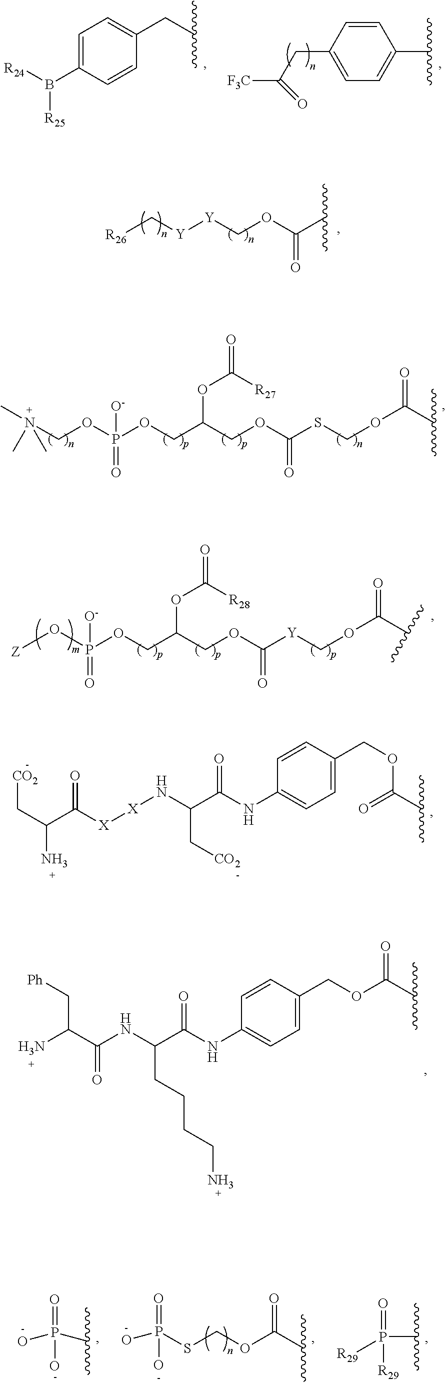

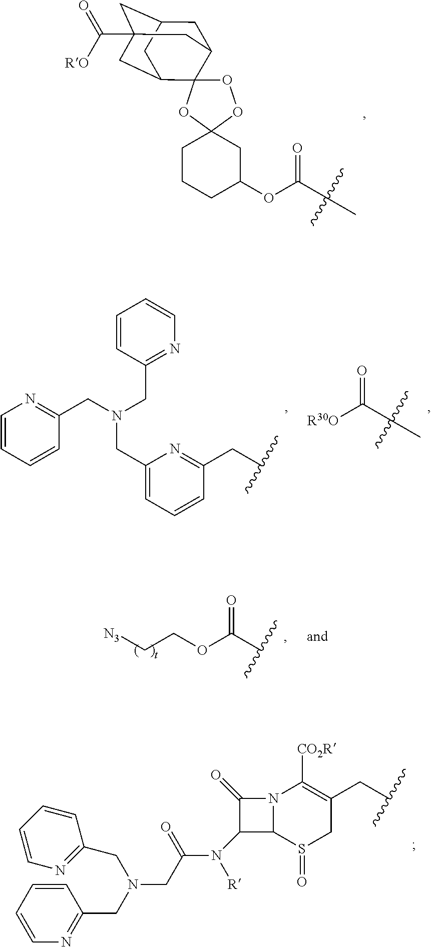

wherein:

m is 0 or 1;

each occurrence of n is independently 2 or 3;

each occurrence of p is independently 1, 2, or 3;

t is 1, 2, or 3;

R₂₄ and R₂₅ are each independently selected from the group consisting of hydroxy, C₁-C₆ alkyl, and C₁-C₆ alkoxy,
or R₂₄ and R₂₅ can combine with the atoms to which they are bound to form a 4-6 membered ring;

R₂₆ is hydroxy or

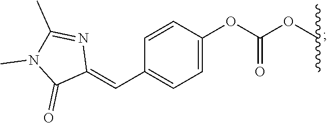 [SMILES: *OC(=O)Oc1ccc(/C=C2\N=C(C)N(C)C2=O)cc1]

R₂₇ and R₂₈ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;

each occurrence of R²⁹ is independently selected from the group consisting of optionally substituted C₁-C₆ alkyl, optionally substituted C₆-C₁₀ aryl, and optionally substituted C₂-C₁₀ heteroaryl;

R³⁰ is optionally substituted C₁-C₁₂ alkyl;

each occurrence of X is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;

Y is selected from the group consisting of —O—, —NH—, and —S—; and

Z is selected from the group consisting of choline, ethanolamine, serine, inositol, glycerol, phosphatidylcholine, lysophosphatidic acid, and glucose;

or a salt, solvate, stereoisomer, or geometric isomer thereof.

Embodiment 2 provides the compound of Embodiment 1, wherein the compound of Formula (II) is a compound of Formula (IIa):

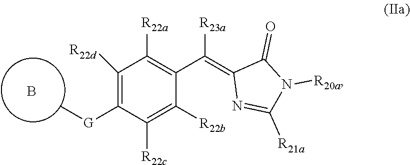

wherein:

G is selected from the group consisting of —O—, —S—, or —NH—;

R₂₀ₐ, R₂₁ₐ, and R₂₃ₐ are each independently selected from the group consisting of hydrogen, deuterium, and optionally substituted C₁-C₆ alkyl;

R^{22a}, R^{22b}, R^{22c}, and R^{22d} are each independently selected from the group consisting of hydrogen, deuterium, tritium, halogen, hydroxy, N(R″)(R″), SR″, sulfide, thiolactone, S(═O)₂OR″, S(═O)R″, cyano, C₁-C₆ alkyl, C₂-C₆ alkenyl, C₂-C₆ alkynyl, nitro, C(═O)N(R″)(R″), P(═O)(OR″)₂, PR″₃, C₆-C₁₂ aryl, C₄-C₁₀ heteroaryl, C₁-C₆ alkoxy, optionally substituted boron, optionally substituted silicon, transition metal, C(═O)OR″, and C(═O)R″;

each occurrence of R″ is independently selected from the group consisting of hydrogen, deuterium, hydroxy, C₁-C₆ alkyl, and C₁-C₆ alkoxy;

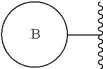

is selected from the group consisting of:

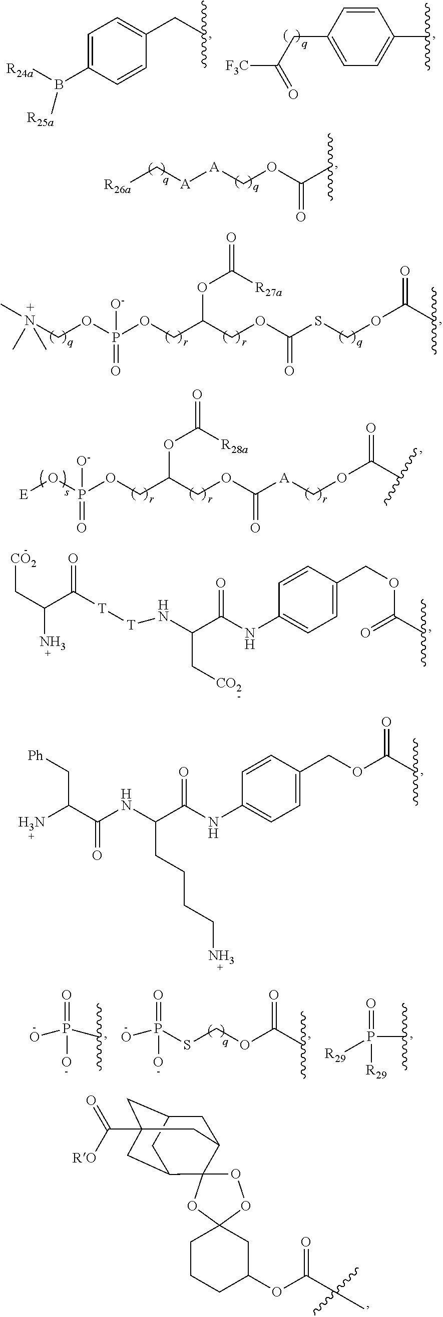

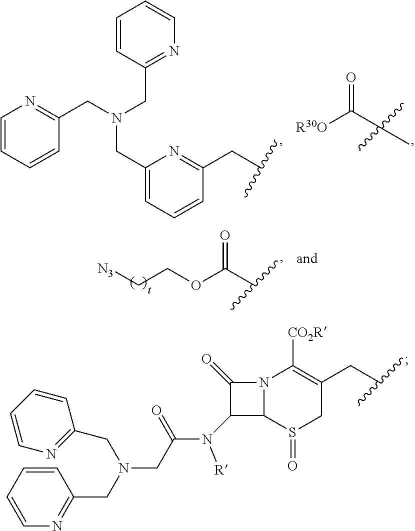

wherein:

each occurrence of q is independently 2 or 3;

each occurrence of r is independently 1, 2, or 3;

s is 0 or 1;

R₂₄ₐ and R₂₅ₐ are each independently selected from the group consisting of hydroxy, C₁-C₆ alkyl, and C₁-C₆ alkoxy,
or R₂₄ₐ and R₂₅ₐ can combine with the atoms to which they are bound to form a 4-6 membered ring;

R₂₆ₐ is hydroxy or

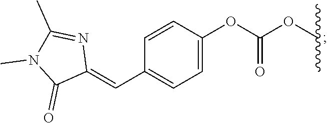 [SMILES: *OC(=O)Oc1ccc(/C=C2\N=C(C)N(C)C2=O)cc1]

R₂₇ₐ and R₂₈ₐ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;

A is selected from the group consisting of —O—, —NH—, and —S—;

E is selected from the group consisting of choline, ethanolamine, serine, inositol, glycerol, phosphatidylcholine, lysophosphatidic acid, and glucose; and

each occurrence of T is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and selenocysteine;

or a salt, solvate, stereoisomer, or geometric isomer thereof:

Embodiment 3 provides the compound of Embodiment 1 or 2, wherein the compound is selected from the group consisting of:

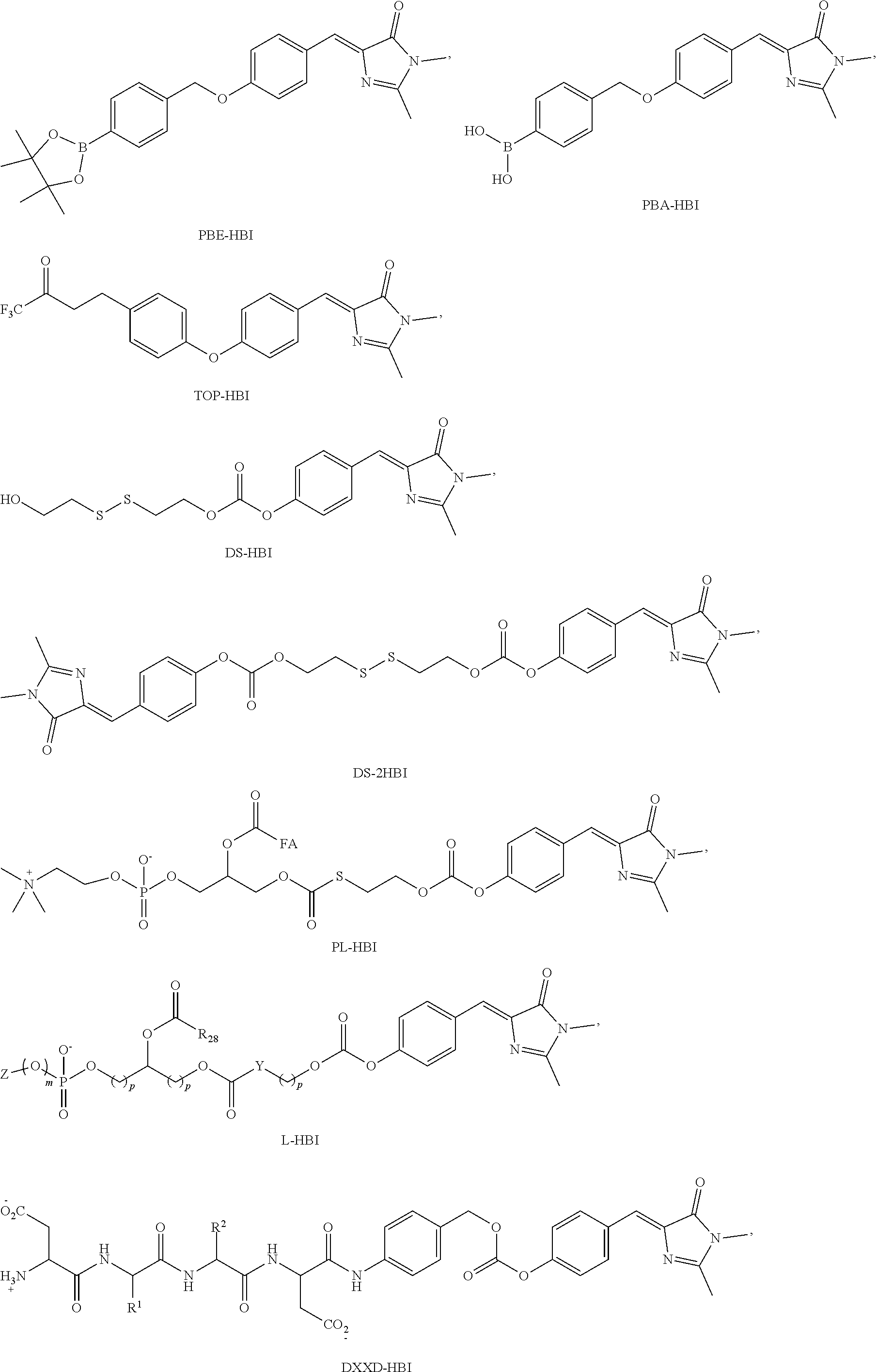

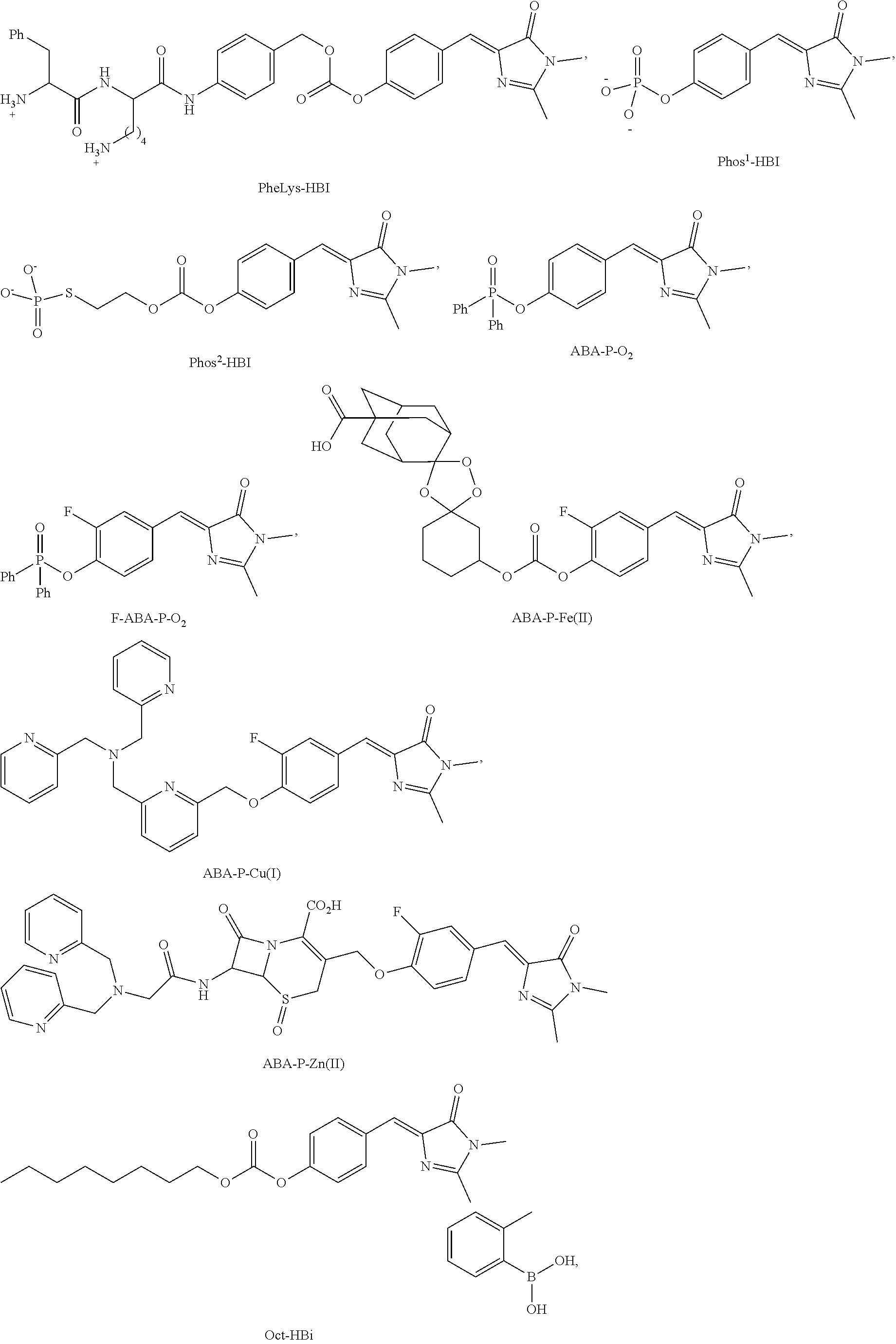

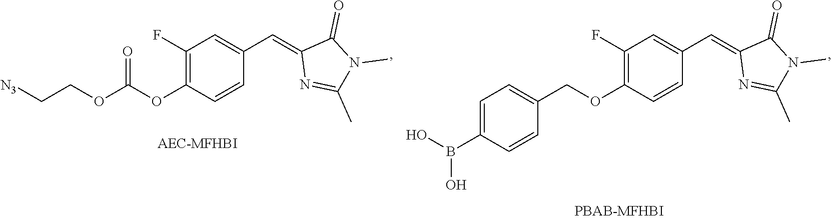 [SMILES: CC1=N/C(=C\c2ccc(OC(=O)OCCN=[N+]=[N-])c(F)c2)C(=O)N1C ; CC1=N/C(=C\c2ccc(OCc3ccc(B(O)O)cc3)c(F)c2)C(=O)N1C]

and combinations thereof;

wherein R¹ and R² are each independently selected from the group consisting of hydrogen, CH₃, CH₂—OH, and CH(OH)—CH₃.

Embodiment 4 provides the compound of any one of Embodiments 1-4, wherein the compound reacts with a biomarker to provide a compound of Formula (I):

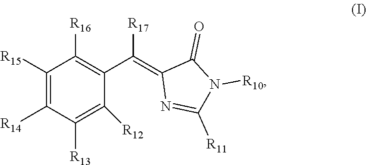

wherein:

R₁₀, R₁₁, and R₁₇ are each independently selected from the group consisting of hydrogen, deuterium, and optionally substituted C₁-C₆ alkyl;

R₁₂, R₁₃, R₁₄, R₁₅, and R₁₆ are each independently selected from the group consisting of hydrogen, deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;

each occurrence of R′ is independently selected from the group consisting of hydrogen, deuterium, hydroxy, C₁-C₆ alkyl, and C₁-C₆ alkoxy,

with the proviso that one or more of R₁₂, R₁₃, R₁₄, R₁₅, and R₁₆ is selected from the group consisting of OH, NHR′, and SH;

or a salt, solvate, stereoisomer, or geometric isomer thereof.

Embodiment 5 provides the compound of Embodiment 4, wherein the biomarker is selected from the group consisting of hydrogen peroxide (H₂O₂), dimethylsulfide (H₂S), superoxide (O₂⁻), peroxynitrite (ONOO⁻), glutathione, hepatic lipase, cathepsin B, the caspase family, alkaline phosphatase, Cu(I), Fe(II), and Zn(II), and combinations thereof.

Embodiment 6 provides a method of detecting a disease or a disorder in a subject in need thereof, the method comprising the steps of:

(a) expressing an RNA sequence comprising an RNA aptamer in the subject;

(b) administering to the subject a compound of Formula (II):

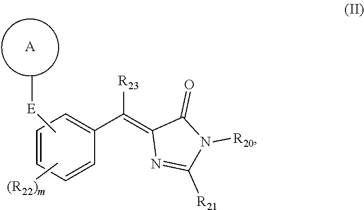

wherein:

R₂₀, R₂₁, and R₂₃ are each independently selected from the group consisting of hydrogen, deuterium, and optionally substituted C₁-C₆ alkyl;

each occurrence of R₂₂ is independently selected from the group consisting of deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;

each occurrence of R′ is independently selected from the group consisting of hydrogen, deuterium, hydroxy, C₁-C₆ alkyl, and C₁-C₆ alkoxy;

m is an integer from 0 to 4;

E is selected from the group consisting of —O—, —S—, and —NH—;

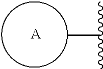

is selected from the group consisting of

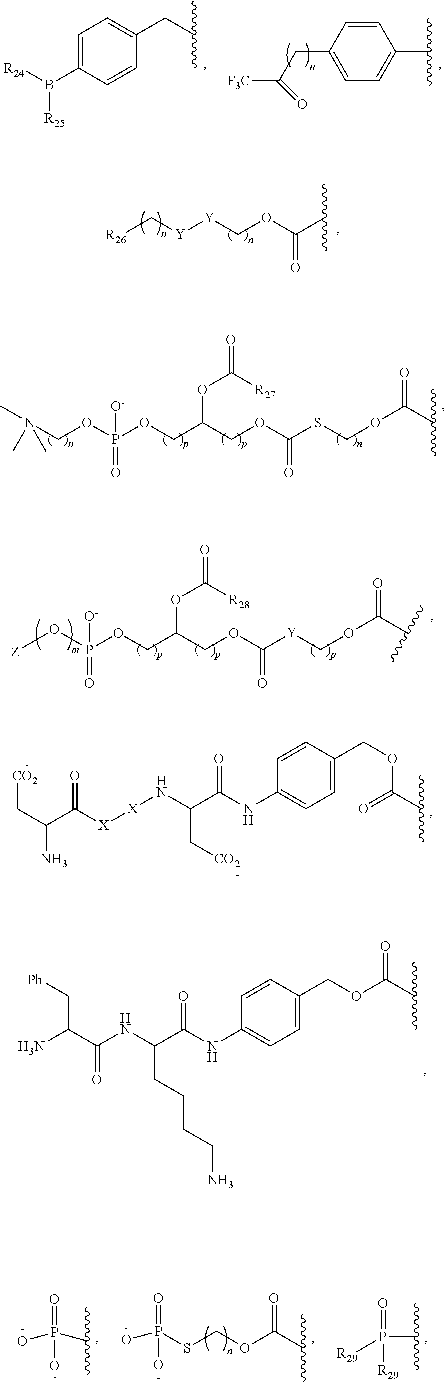

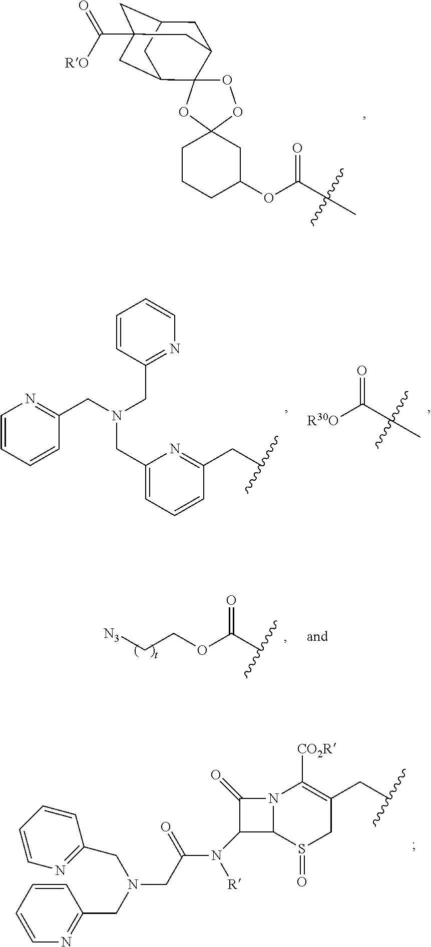

wherein:

m is 0 or 1;

each occurrence of n is independently 2 or 3;

each occurrence of p is independently 1, 2, or 3;

t is 1, 2, or 3;

R₂₄ and R₂₅ are each independently selected from the group consisting of hydroxy, C₁-C₆ alkyl, and C₁-C₆ alkoxy,
or R₂₄ and R₂₅ can combine with the atoms to which they are bound to form a 4-6 membered ring;

R₂₆ is hydroxy or

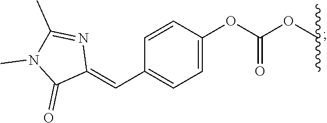 [SMILES: *OC(=O)Oc1ccc(/C=C2\N=C(C)N(C)C2=O)cc1]

R₂₇ and R₂₈ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;

each occurrence of R²⁹ is independently selected from the group consisting of optionally substituted C₁-C₆ alkyl, optionally substituted C₆-C₁₀ aryl, and optionally substituted C₂-C₁₀ heteroaryl;

R³⁰ is optionally substituted C₁-C₁₂ alkyl;

each occurrence of X is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;

Y is selected from the group consisting of —O—, —NH—, and —S—; and

Z is selected from the group consisting of choline, ethanolamine, serine, inositol, glycerol, phosphatidylcholine, lysophosphatidic acid, and glucose;

or a salt, solvate, stereoisomer, or geometric isomer thereof;

and

(c) detecting fluorescence emission associated with the RNA aptamer.

Embodiment 7 provides the method of Embodiment 6, wherein the RNA aptamer is selected from the group consisting of Spinach aptamer, Baby Spinach aptamer, Corn aptamer, and Broccoli aptamer.

Embodiment 8 provides the method of Embodiment 6 or 7, wherein the compound of Formula (II) is a compound of Formula (IIa):

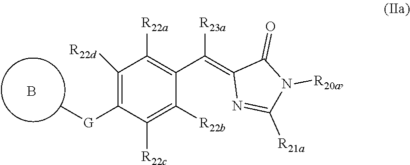

wherein:

G is selected from the group consisting of —O—, —S—, or —NH—;

R₂₀ₐ, R₂₁ₐ, and R₂₃ₐ are each independently selected from the group consisting of hydrogen, deuterium, and optionally substituted C₁-C₆ alkyl;

R^{22a}, R^{22b}, R^{22c}, and R^{22a} are each independently selected from the group consisting of hydrogen, deuterium, tritium, halogen, hydroxy, N(R″)(R″), SR″, sulfide, thiolactone, S(═O)₂OR″, S(═O)R″, cyano, C₁-C₆ alkyl, C₂-C₆ alkenyl, C₂-C₆ alkynyl, nitro, C(═O)N(R″)(R″), P(═O)(OR″)₂, PR″₃, C₆-C₁₂ aryl, C₄-C₁₀ heteroaryl, C₁-C₆ alkoxy, optionally substituted boron, optionally substituted silicon, transition metal, C(═O)OR″, and C(═O)R″;

each occurrence of R″ is independently selected from the group consisting of hydrogen, deuterium, hydroxy, C₁-C₆ alkyl, and C₁-C₆ alkoxy;

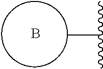

is selected from the group consisting of

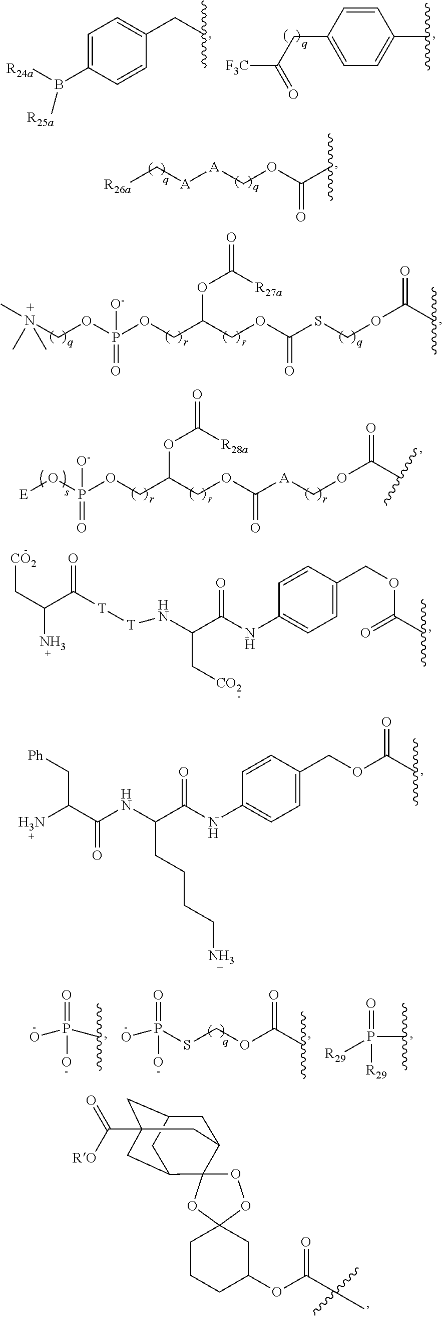

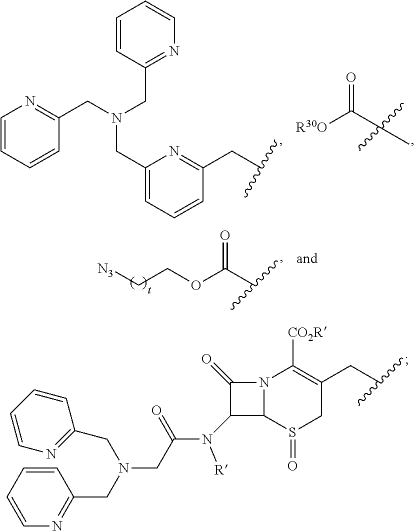

wherein:

each occurrence of q is independently 2 or 3;

each occurrence of r is independently 1, 2, or 3;

s is 0 or 1;

R₂₄ₐ and R₂₅ₐ are each independently selected from the group consisting of hydroxy, C₁-C₆ alkyl, and C₁-C₆ alkoxy,
or R₂₄ₐ and R₂₅ₐ can combine with the atoms to which they are bound to form a 4-6 membered ring;

R₂₆ₐ is hydroxy or

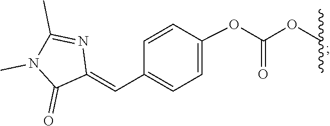 [SMILES: *OC(=O)Oc1ccc(/C=C2\N=C(C)N(C)C2=O)cc1]

R₂₇ₐ and R₂₈ₐ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;

A is selected from the group consisting of —O—, —NH—, and —S—;

E is selected from the group consisting of choline, ethanolamine, serine, inositol, glycerol, phosphatidylcholine, lysophosphatidic acid, and glucose; and

each occurrence of T is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and selenocysteine;

or a salt, solvate, stereoisomer, or geometric isomer thereof.

Embodiment 9 provides the method of any one of Embodiments 6-8, wherein the compound is selected from the group consisting of:

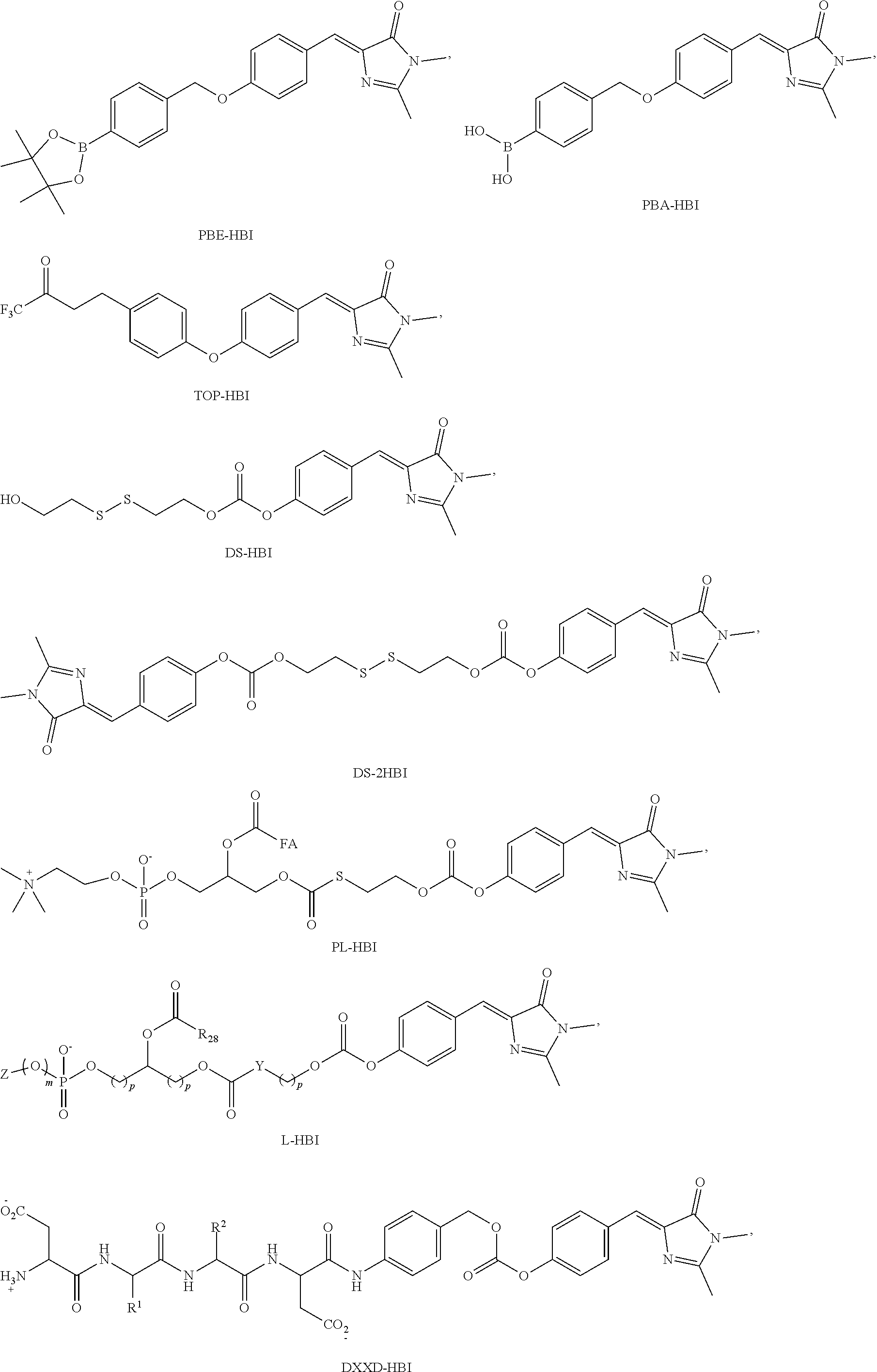

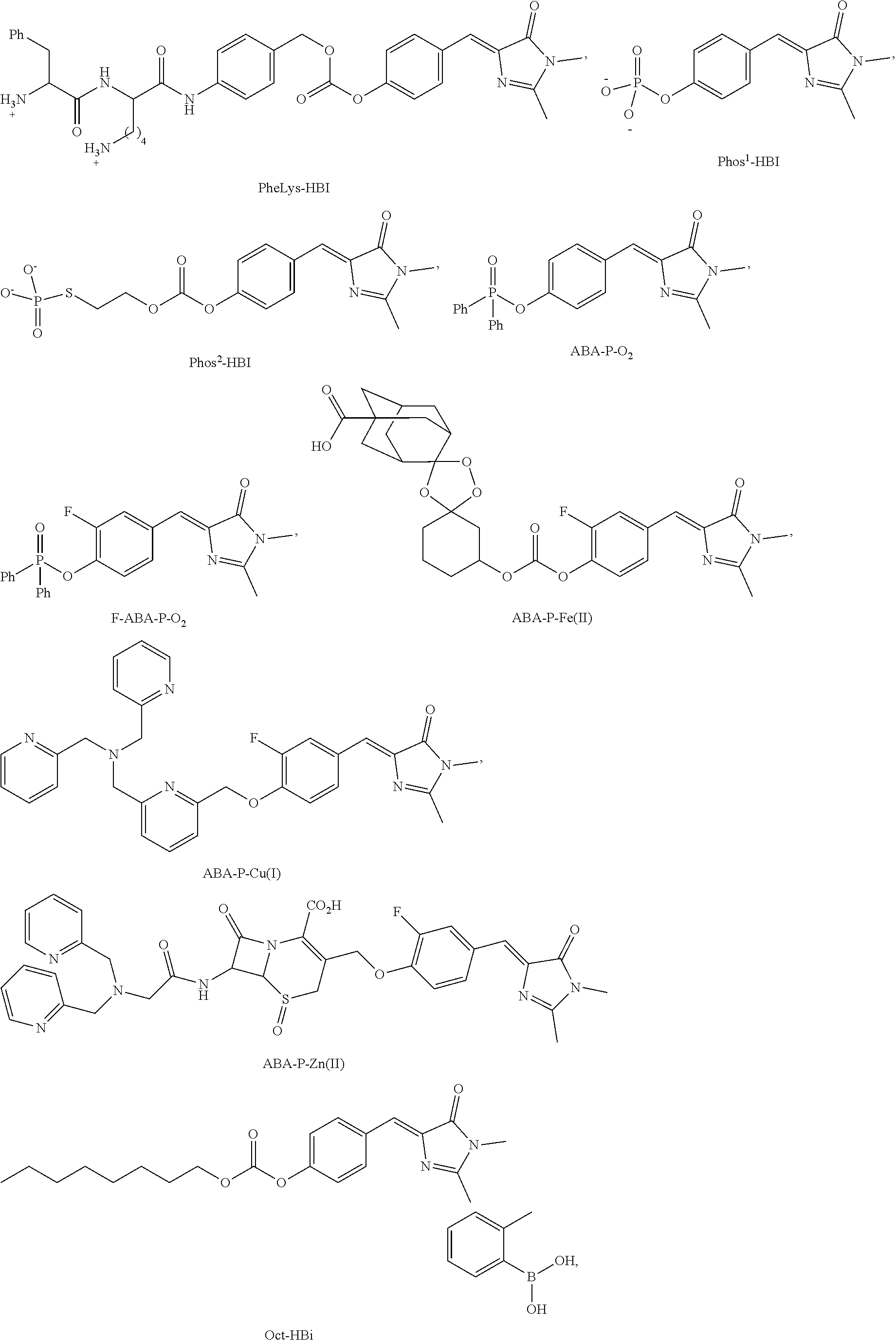

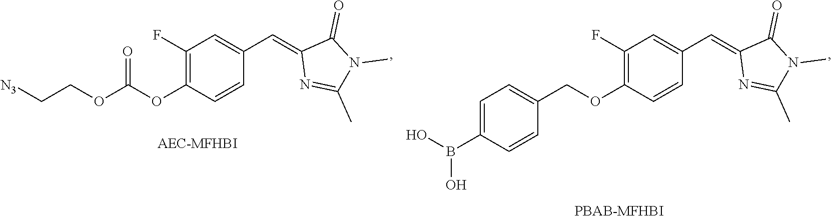 [SMILES: CC1=N/C(=C\c2ccc(OC(=O)OCCN=[N+]=[N-])c(F)c2)C(=O)N1C ; CC1=N/C(=C\c2ccc(OCc3ccc(B(O)O)cc3)c(F)c2)C(=O)N1C]

and combinations thereof;

wherein R¹ and R² are each independently selected from the group consisting of hydrogen, CH₃, CH₂—OH, and CH(OH)—CH₃.

Embodiment 10 provides the method of any one of Embodiments 6-9, wherein administering to the subject a compound of Formula (II) further comprises deprotecting the compound of Formula (II) via a reaction with a biomarker, producing a fluorogenic ligand.

Embodiment 11 provides the method of Embodiment 10, wherein the biomarker is selected from the group consisting of hydrogen peroxide, dimethylsulfide, superoxide, hydroxyl radical, hydroxide anion, peroxynitrite, nitrogen dioxide, nitrosoperoxycarbonate, dinitrogen trioxide, aldehyde, glutathione, glutathione-synthesizing enzymes, lipases, cathepsin B, the caspase family, acid phosphatase, alkaline phosphatase, Cu(I), Fe(II), and Zn(II), and combinations thereof.

Embodiment 12 provides the method of Embodiment 10 or 11, wherein the fluorogenic ligand is a compound of Formula (I):

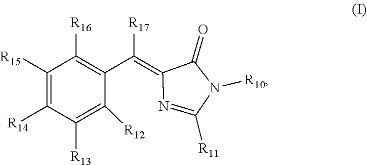

wherein:

R₁₀, R₁₁, and R₁₇ are each independently selected from the group consisting of hydrogen, deuterium, and optionally substituted C₁-C₆ alkyl;

R₁₂, R₁₃, R₁₄, R₁₅, and R₁₆ are each independently selected from the group consisting of hydrogen, deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;

each occurrence of R′ is independently selected from the group consisting of hydrogen, deuterium, hydroxy, C₁-C₆ alkyl, and C₁-C₆ alkoxy,

with the proviso that one or more of R₁₂, R₁₃, R₁₄, R₁₅, and R₁₆ is selected from the group consisting of OH, NHR′, and SH;

or a salt, solvate, stereoisomer, or geometric isomer thereof.

Embodiment 13 provides the method of any one of Embodiments 10-12, wherein deprotecting the compound of Formula (II) via a reaction with a biomarker further comprises binding the fluorogenic ligand to the RNA aptamer.

Embodiment 14 provides the method of Embodiment 13, wherein binding the fluorogenic ligand to the RNA aptamer leads to fluorescence emission associated with the aptamer.

Embodiment 15 provides the method of Embodiment 14, wherein the fluorescence emission associated with the aptamer indicates that the subject has a disease or disorder associated with the biomarker.

Embodiment 16 provides a method of detecting a disease or a disorder in a biological sample, the method comprising:

(a) providing a chip comprising a grafted RNA aptamer;

(b) contacting the chip with a biological sample;

(c) contacting the biological sample with a compound of Formula (II):

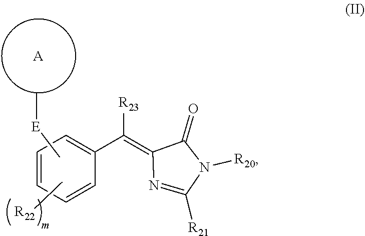

wherein:

R₂₀, R₂₁, and R₂₃ are each independently selected from the group consisting of hydrogen, deuterium, and optionally substituted C₁-C₆ alkyl;

each occurrence of R₂₂ is independently selected from the group consisting of deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;

each occurrence of R′ is independently selected from the group consisting of hydrogen, deuterium, hydroxy, C₁-C₆ alkyl, and C₁-C₆ alkoxy;

m is an integer from 0 to 4;

E is selected from the group consisting of —O—, —S—, and —NH—;

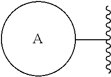

is selected from the group consisting of

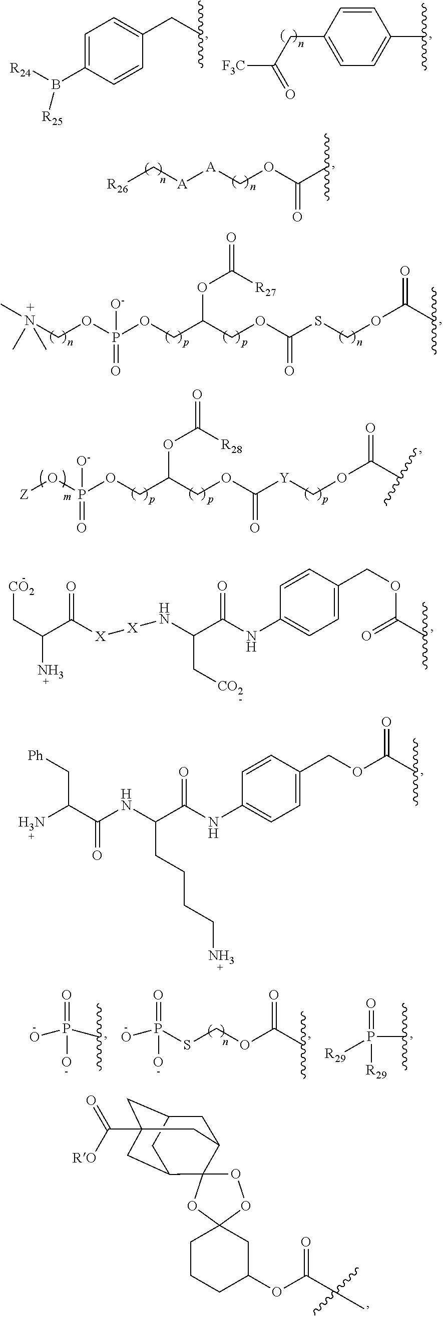

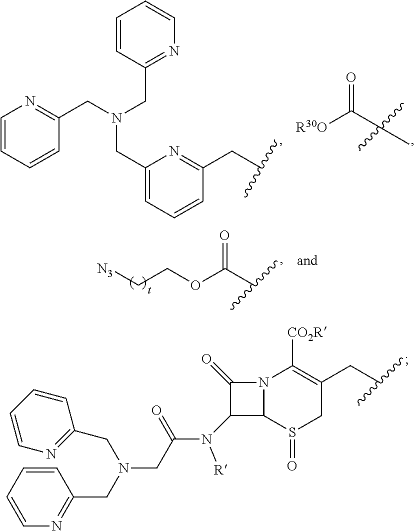

wherein:

m is 0 or 1;

each occurrence of n is independently 2 or 3;

each occurrence of p is independently 1, 2, or 3;

t is 1, 2, or 3;

R₂₄ and R₂₅ are each independently selected from the group consisting of hydroxy, C₁-C₆ alkyl, and C₁-C₆ alkoxy,
or R₂₄ and R₂₅ can combine with the atoms to which they are bound to form a 4-6 membered ring;

R₂₆ is hydroxy or

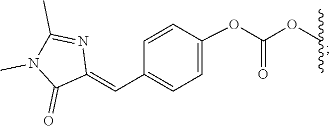 [SMILES: *OC(=O)Oc1ccc(/C=C2\N=C(C)N(C)C2=O)cc1]

R₂₇ and R₂₈ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;

each occurrence of R²⁹ is independently selected from the group consisting of optionally substituted C₁-C₆ alkyl, optionally substituted C₆-C₁₀ aryl, and optionally substituted C₂-C₁₀ heteroaryl;

R³⁰ is optionally substituted C₁-C₁₂ alkyl;

each occurrence of X is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;

Y is selected from the group consisting of —O—, —NH—, and —S—; and

Z is selected from the group consisting of choline, ethanolamine, serine, inositol, glycerol, phosphatidylcholine, lysophosphatidic acid, and glucose;

or a salt, solvate, stereoisomer, or geometric isomer thereof;

(d) rinsing the chip; and

(e) detecting fluorescence emission from the RNA aptamer.

Embodiment 17 provides the method of Embodiment 16, wherein the RNA aptamer is Spinach aptamer, Baby Spinach aptamer, Corn aptamer, or Broccoli aptamer.

Embodiment 18 provides the method of Embodiment 16 or 17, wherein the compound of Formula (II) is a compound of Formula (IIa):

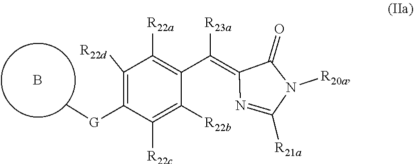

wherein:

G is selected from the group consisting of —O—, —S—, or —NH—;

R₂₀ₐ, R₂₁ₐ, and R₂₃ₐ are each independently selected from the group consisting of hydrogen, deuterium, and optionally substituted C₁-C₆ alkyl;

R^{22a}, R^{22b}, R^{22c}, and R^{22d} are each independently selected from the group consisting of hydrogen, deuterium, tritium, halogen, hydroxy, N(R″)(R″), SR″, sulfide, thiolactone, S(═O)₂OR″, S(═O)R″, cyano, C₁-C₆ alkyl, C₂-C₆ alkenyl, C₂-C₆ alkynyl, nitro, C(═O)N(R″)(R″), P(═O)(OR″)₂, PR″₃, C₆-C₁₂ aryl, C₄-C₁₀ heteroaryl, C₁-C₆ alkoxy, optionally substituted boron, optionally substituted silicon, transition metal, C(═O)OR″, and C(═O)R″;

each occurrence of R″ is independently selected from the group consisting of hydrogen, deuterium, hydroxy, C₁-C₆ alkyl, and C₁-C₆ alkoxy;

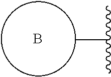

is selected from the group consisting of

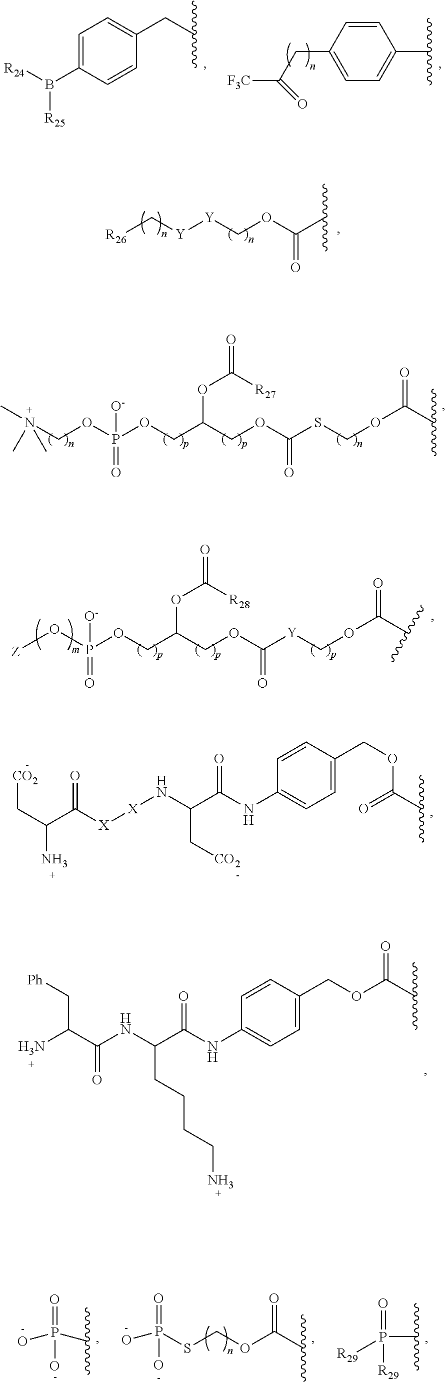

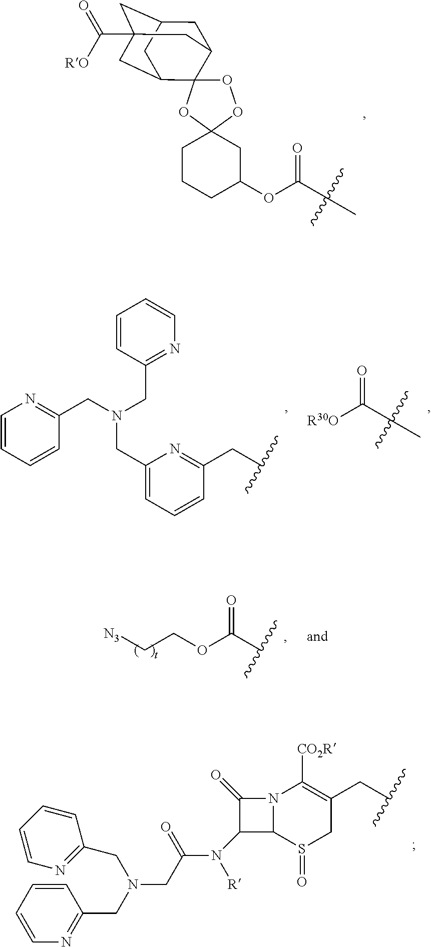

wherein:

each occurrence of q is independently 2 or 3;

each occurrence of r is independently 1, 2, or 3;

s is 0 or 1;

R₂₄ₐ and R₂₅ₐ are each independently selected from the group consisting of hydroxy, C₁-C₆ alkyl, and C₁-C₆ alkoxy,
or R₂₄ₐ and R₂₅ₐ can combine with the atoms to which they are bound to form a 4-6 membered ring;

R₂₆ₐ is hydroxy or

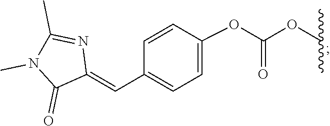 [SMILES: *OC(=O)Oc1ccc(/C=C2\N=C(C)N(C)C2=O)cc1]

R₂₇ₐ and R₂₈ₐ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;

A is selected from the group consisting of —O—, —NH—, and —S—;

E is selected from the group consisting of choline, ethanolamine, serine, inositol, glycerol, phosphatidylcholine, lysophosphatidic acid, and glucose; and

each occurrence of T is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and selenocysteine;

or a salt, solvate, stereoisomer, or geometric isomer thereof.

Embodiment 19 provides the method of any one of Embodiments 16-18, wherein the compound is selected from the group consisting of:

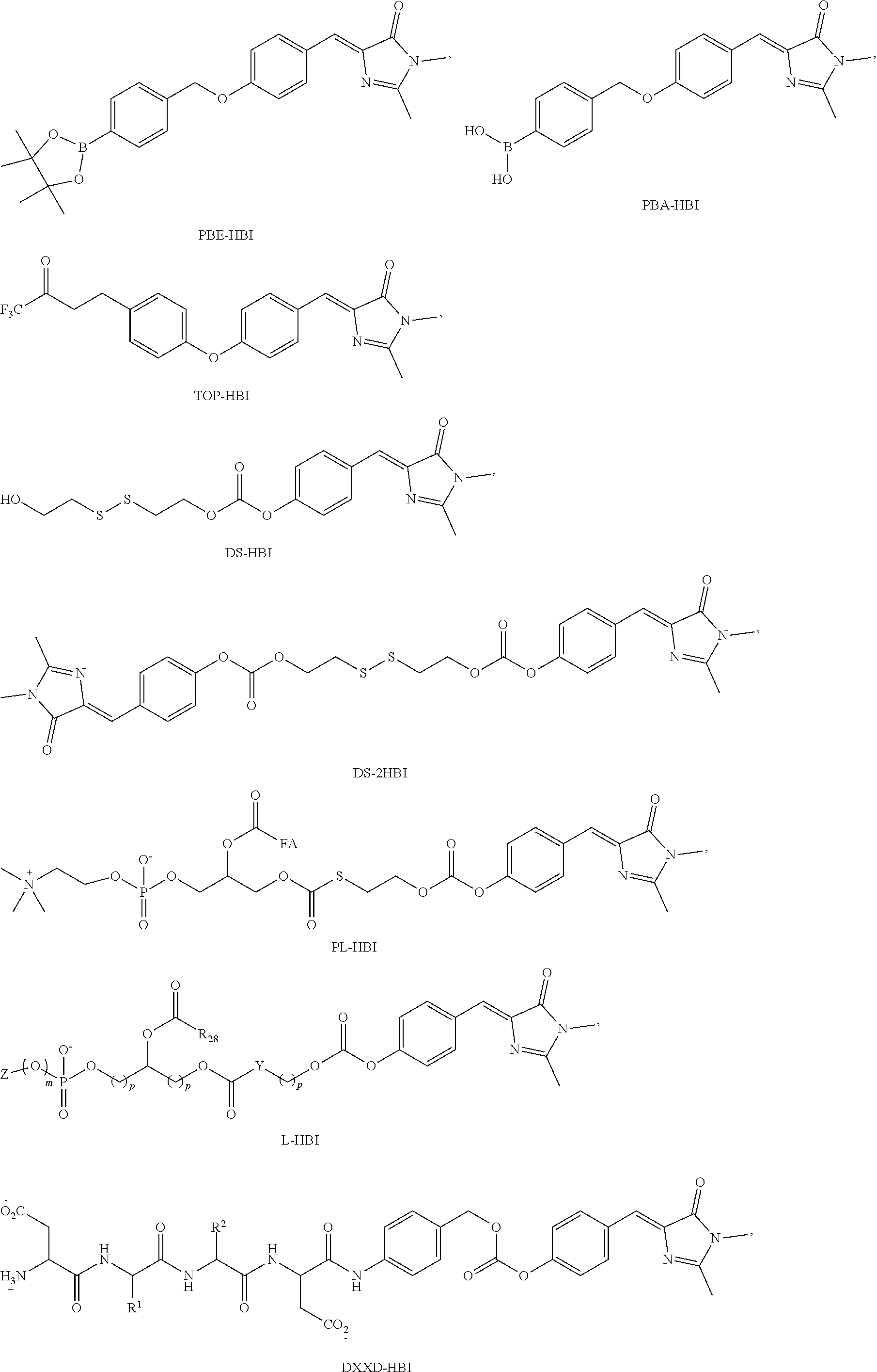

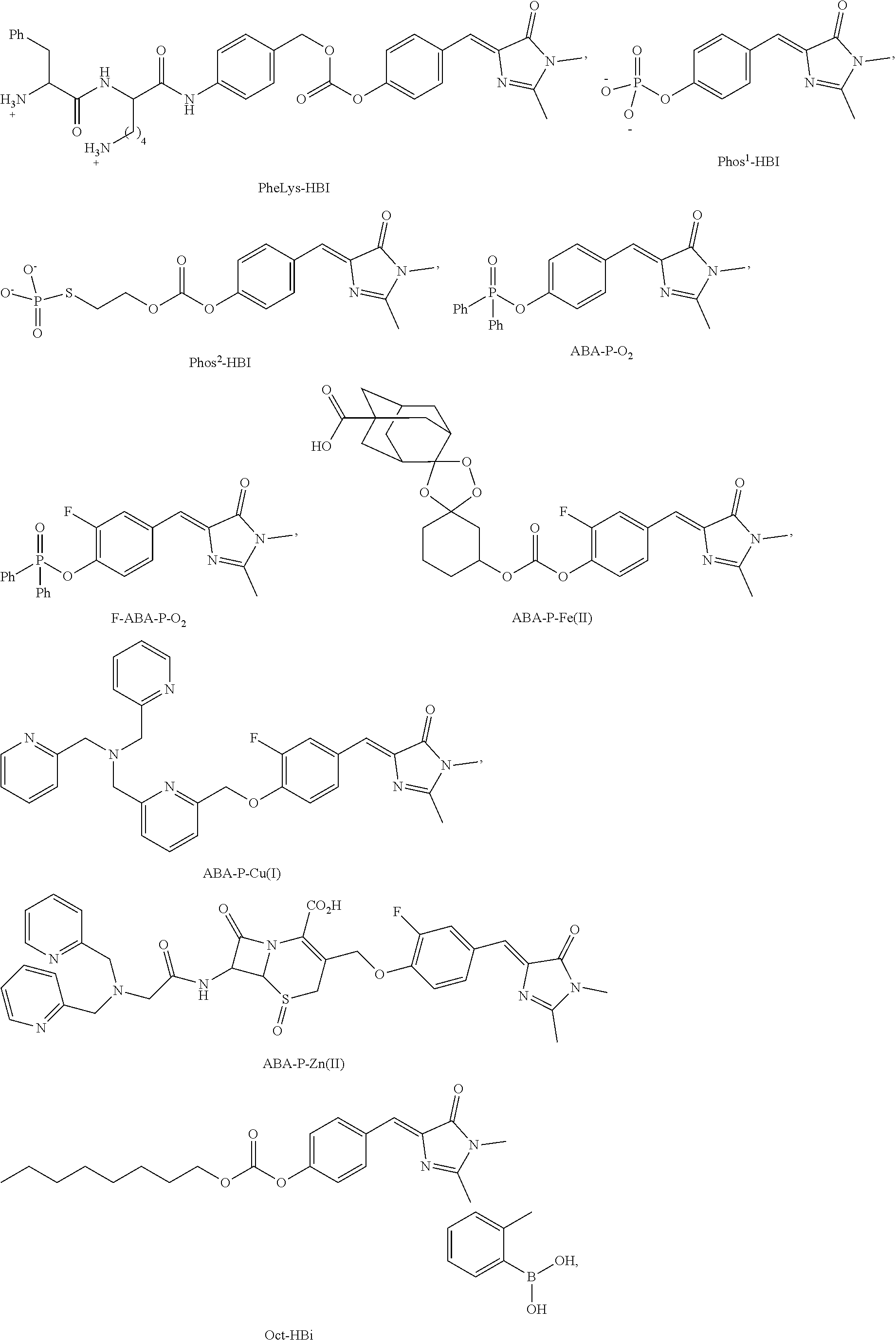

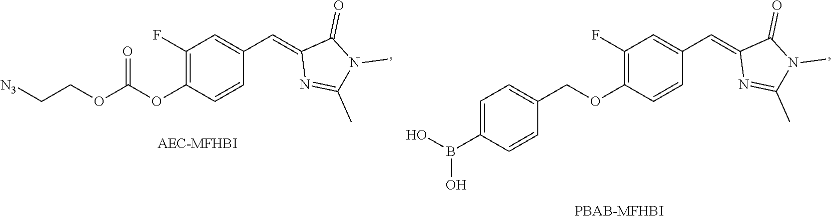 [SMILES: CC1=N/C(=C\c2ccc(OC(=O)OCCN=[N+]=[N-])c(F)c2)C(=O)N1C ; CC1=N/C(=C\c2ccc(OCc3ccc(B(O)O)cc3)c(F)c2)C(=O)N1C]

and combinations thereof;

wherein R¹ and R² are each independently selected from the group consisting of hydrogen, CH₃, CH₂—OH, and CH(OH)—CH₃.

Embodiment 20 provides the method of any one of Embodiments 16-19, wherein contacting the biological sample with a compound of Formula (II) further comprises deprotecting the compound of Formula (II) via a reaction with a biomarker present in the biological sample, producing a fluorogenic ligand, wherein the fluorogenic ligand is optionally a compound of Formula (I):

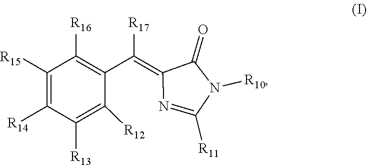

wherein:

R₁₀, R₁₁, and R₁₇ are each independently selected from the group consisting of hydrogen, deuterium, and optionally substituted C₁-C₆ alkyl;

R₁₂, R₁₃, R₁₄, R₁₅, and R₁₆ are each independently selected from the group consisting of hydrogen, deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;

each occurrence of R′ is independently selected from the group consisting of hydrogen, deuterium, hydroxy, C₁-C₆ alkyl, and C₁-C₆ alkoxy,

with the proviso that one or more of R₁₂, R₁₃, R₁₄, R₁₅, and R₁₆ is selected from the group consisting of OH, NHR′, and SH;

or a salt, solvate, stereoisomer, or geometric isomer thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the disclosure. The appended claims are intended to be construed to include all such embodiments and equivalent variations.

What is claimed is:

1. A compound of Formula (II): 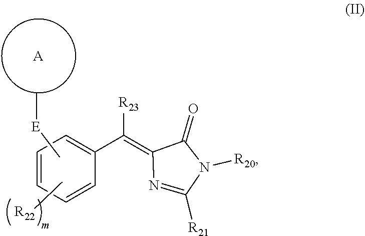
wherein:
R₂₀, R₂₁, and R₂₃ are each independently selected from the group consisting of hydrogen, deuterium, and optionally substituted C₁-C₆ alkyl;
each occurrence of R₂₂ is independently selected from the group consisting of deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;
each occurrence of R′ is independently selected from the group consisting of hydrogen, deuterium, hydroxy, C₁-C₆ alkyl, and C₁-C₆ alkoxy;
m is 0, 1, 2, 3, or 4;
E is selected from the group consisting of —O—, —S—, and —NH—; 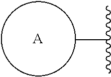
is selected from the group consisting of 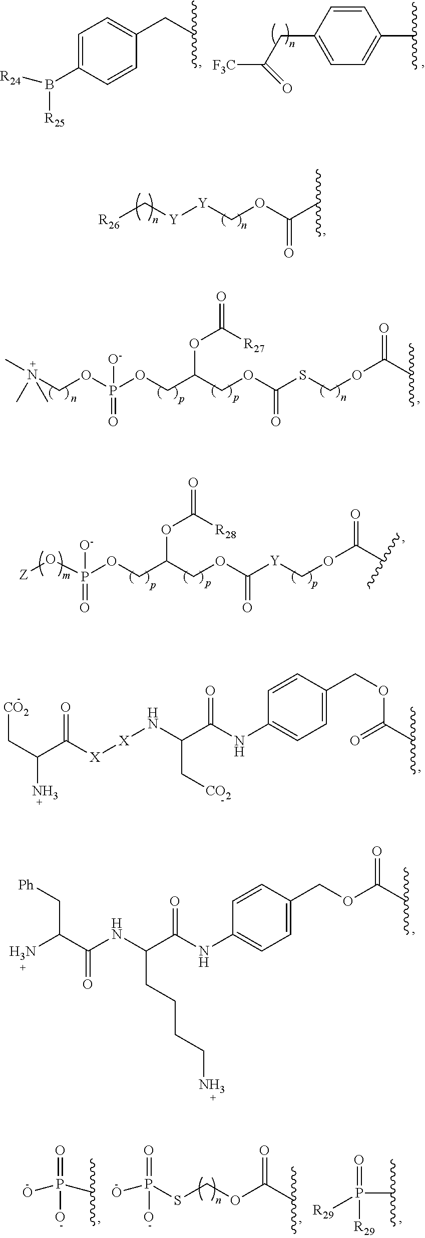 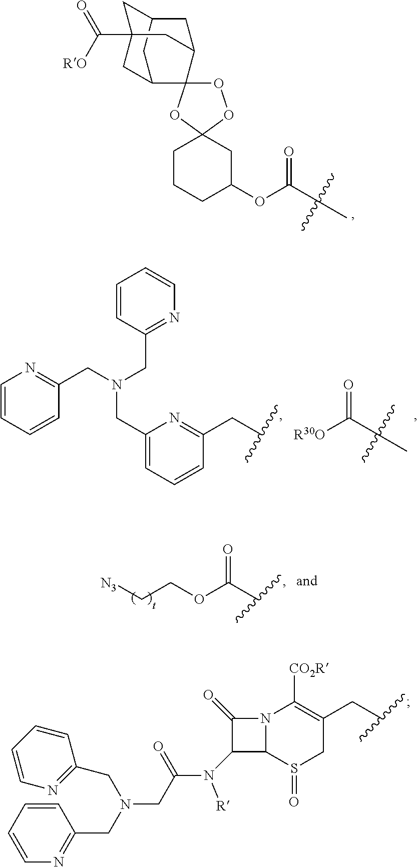
wherein:
m is 0 or 1;
each occurrence of n is independently 2 or 3;
each occurrence of p is independently 1, 2, or 3;
t is 1, 2, or 3;
R₂₄ and R₂₅ are each independently selected from the group consisting of hydroxy, C₁-C₆ alkyl, and C₁-C₆ alkoxy,
or R₂₄ and R₂₅ can combine with the atoms to which they are bound to form a 4-6 membered ring;
R₂₆ is hydroxy or 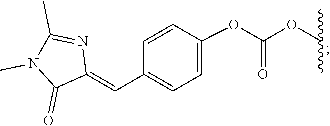 [SMILES: *OC(=O)Oc1ccc(/C=C2\N=C(C)N(C)C2=O)cc1]
R₂₇ and R₂₈ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;
each occurrence of R²⁹ is independently selected from the group consisting of optionally substituted C₁-C₆ alkyl, optionally substituted C₆-C₁₀ aryl, and optionally substituted C₂-C₁₀ heteroaryl;
R³⁰ is optionally substituted C₁-C₁₂ alkyl;
each occurrence of X is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;
Y is selected from the group consisting of —O—, —NH—, and —S—; and
Z is selected from the group consisting of choline, ethanolamine, serine, inositol, glycerol, phosphatidylcholine, lysophosphatidic acid, and glucose;
or a salt, solvate, stereoisomer, or geometric isomer thereof.

2. The compound of claim 1, wherein the compound of Formula (II) is a compound of Formula (IIa): 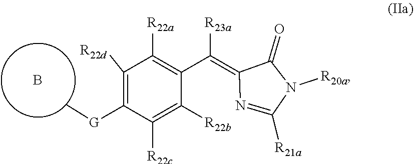
wherein:
G is selected from the group consisting of —O—, —S—, or —NH—;
R₂₀ₐ, R₂₁ₐ, and R₂₃ₐ are each independently selected from the group consisting of hydrogen, deuterium, and optionally substituted C₁-C₆ alkyl;
R^{22a}, R^{22b}, R^{22c}, and R^{22d} are each independently selected from the group consisting of hydrogen, deuterium, tritium, halogen, hydroxy, N(R″)(R″), SR″, sulfide, thiolactone, S(═O)₂OR″, S(═O)R″, cyano, C₁-C₆ alkyl, C₂-C₆ alkenyl, C₂-C₆ alkynyl, nitro, C(═O)N(R″)(R″), P(═O)(OR″)₂, PR″₃, C₆-C₁₂ aryl, C₄-C₁₀ heteroaryl, C₁-C₆ alkoxy, optionally substituted boron, optionally substituted silicon, transition metal, C(═O)OR″, and C(═O)R″;
each occurrence of R″ is independently selected from the group consisting of hydrogen, deuterium, hydroxy, C₁-C₆ alkyl, and C₁-C₆ alkoxy; 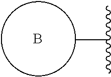
is selected from the group consisting of: 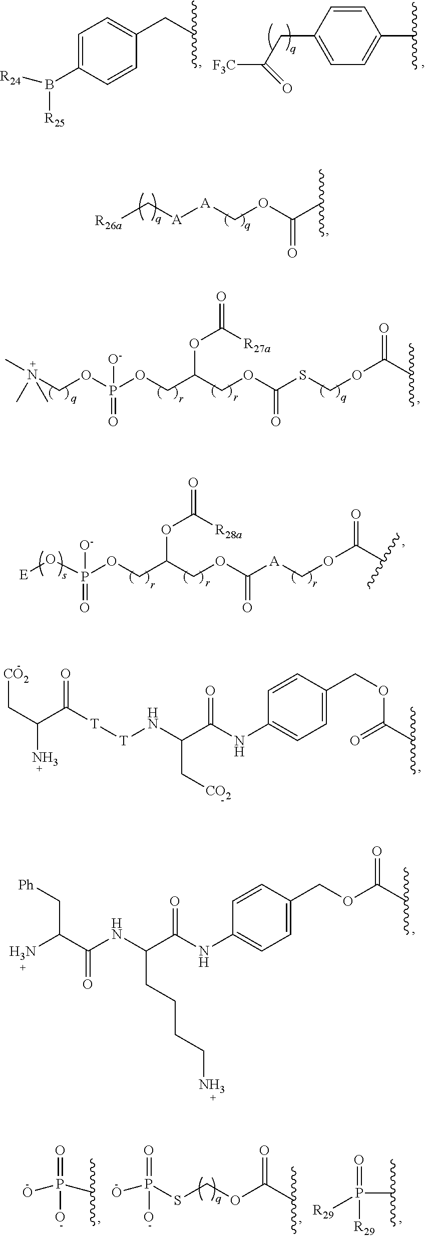 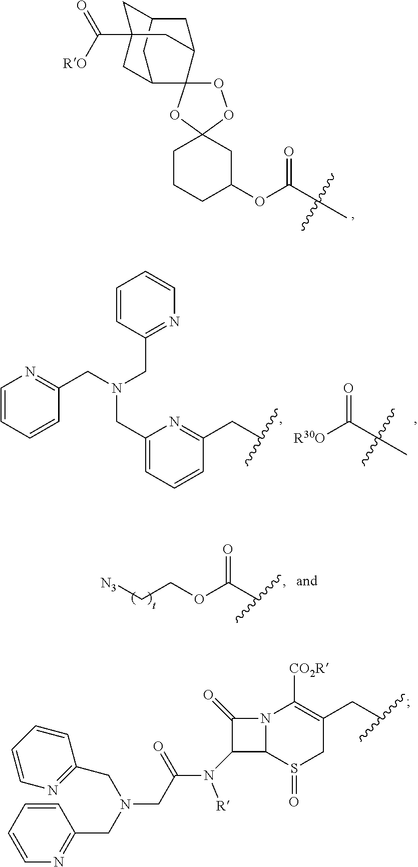
wherein:
each occurrence of q is independently 2 or 3;
each occurrence of r is independently 1, 2, or 3;
s is 0 or 1;
R₂₄ₐ and R₂₅ₐ are each independently selected from the group consisting of hydroxy, C₁-C₆ alkyl, and C₁-C₆ alkoxy,
or R₂₄ₐ and R₂₅ₐ can combine with the atoms to which they are bound to form a 4-6 membered ring;
R₂₆ₐ is hydroxy or 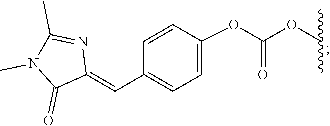 [SMILES: *OC(=O)Oc1ccc(/C=C2\N=C(C)N(C)C2=O)cc1]
R₂₇ₐ and R₂₈ₐ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;
A is selected from the group consisting of —O—, —NH—, and —S—;
E is selected from the group consisting of choline, ethanolamine, serine, inositol, glycerol, phosphatidylcholine, lysophosphatidic acid, and glucose; and
each occurrence of T is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and selenocysteine;
or a salt, solvate, stereoisomer, or geometric isomer thereof:

3. The compound of claim 1, wherein the compound is selected from the group consisting of: 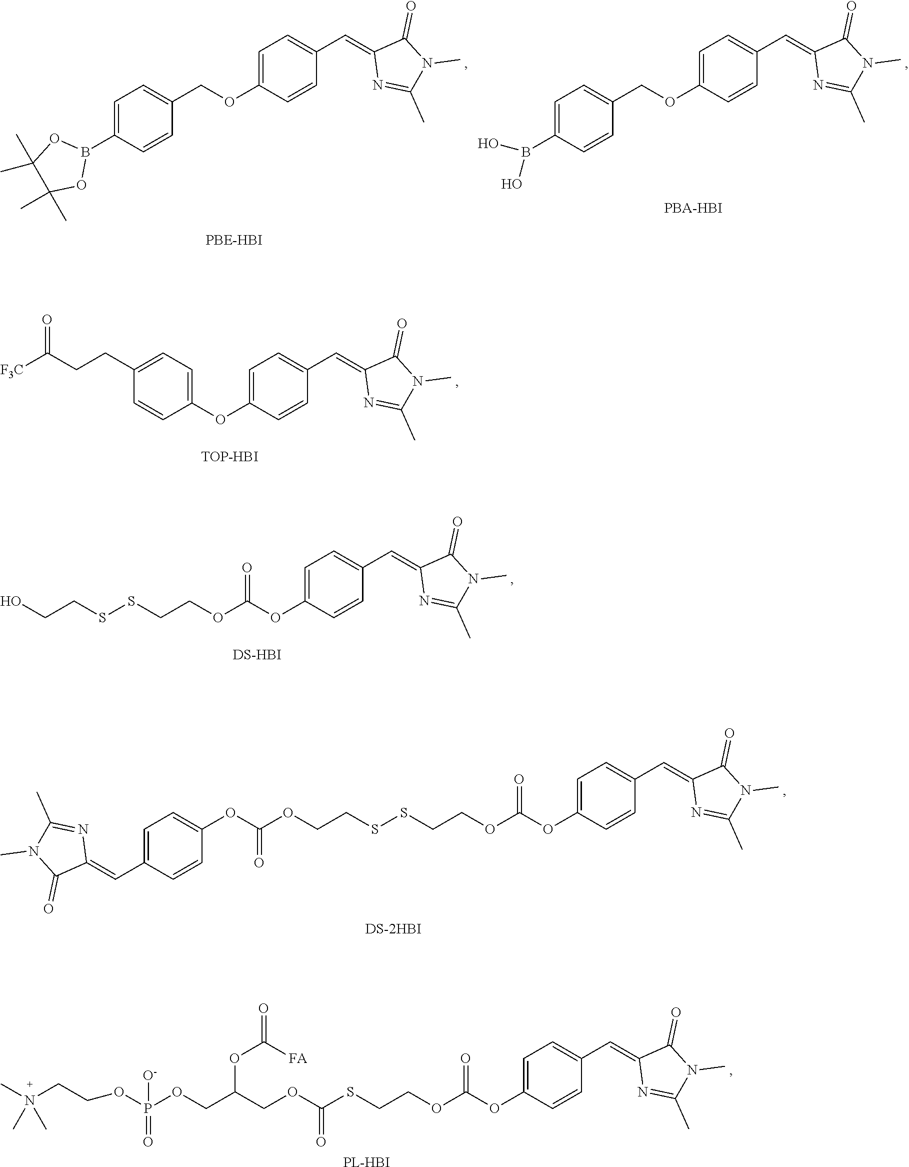 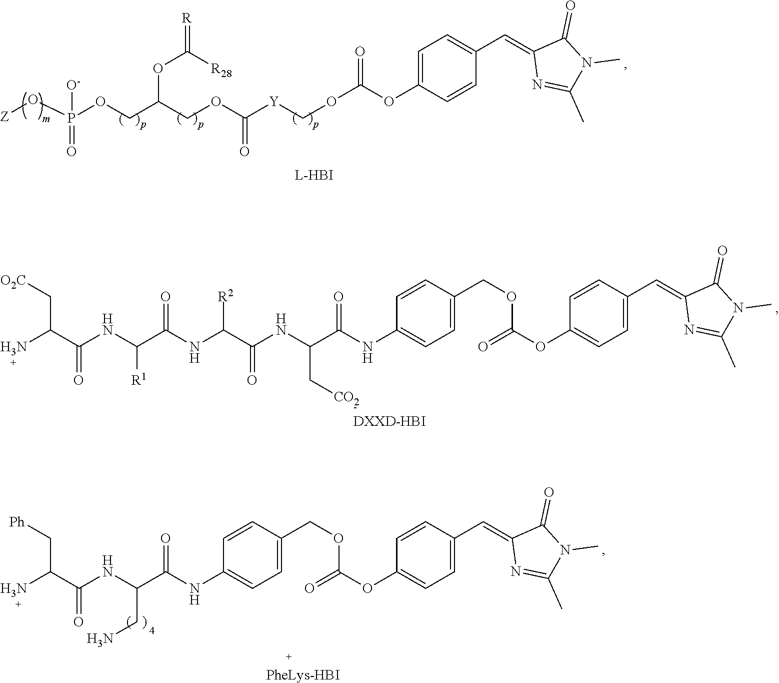 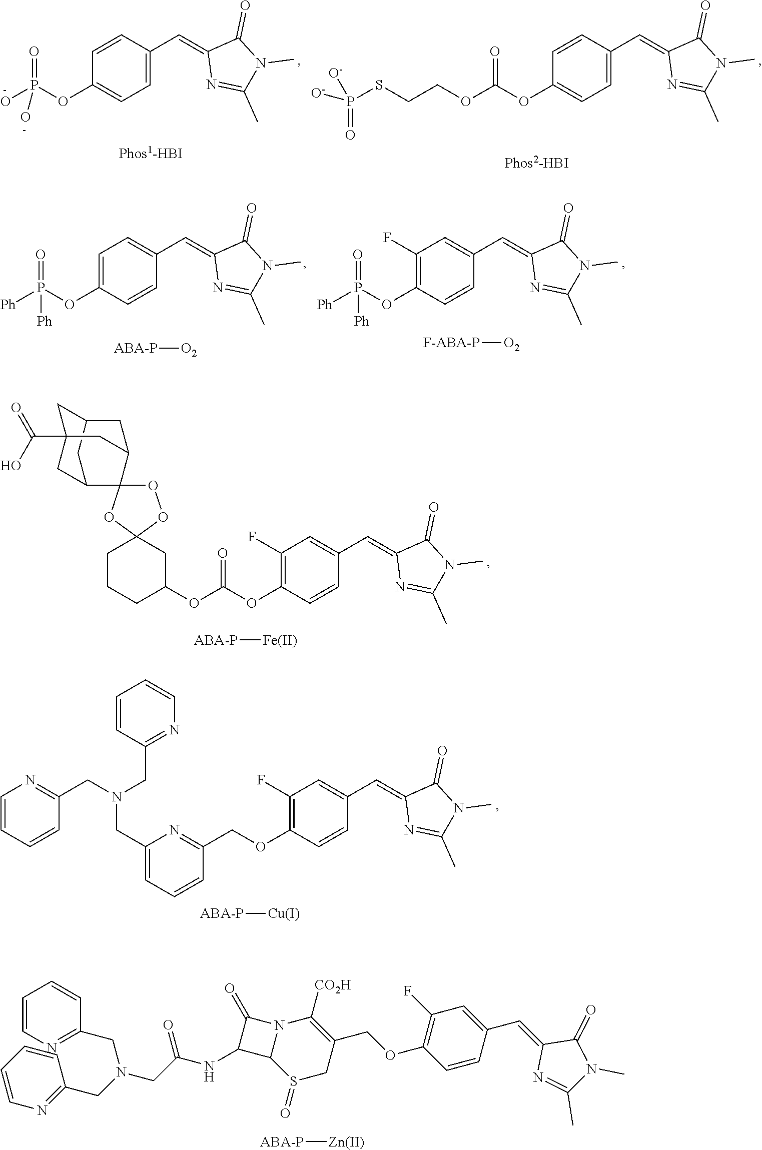 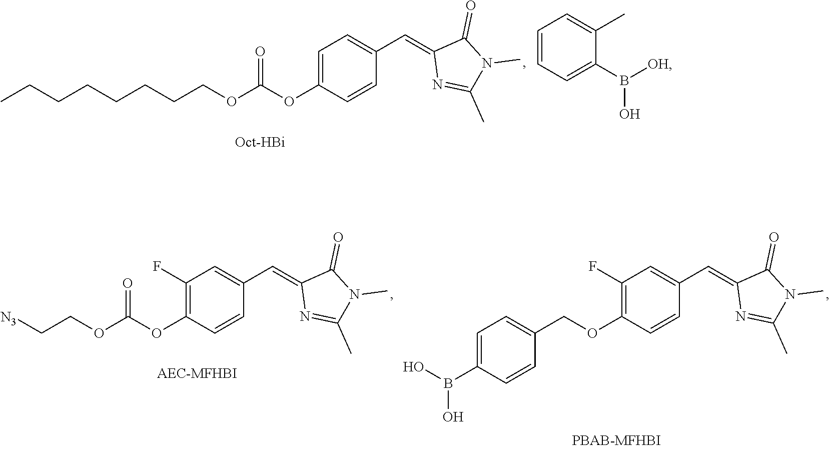
and combinations thereof;
wherein R¹ and R² are each independently selected from the group consisting of hydrogen, CH₃, CH₂—OH, and CH(OH)—CH₃.

4. The compound of claim 1, wherein the compound reacts with a biomarker to provide a compound of Formula (I): 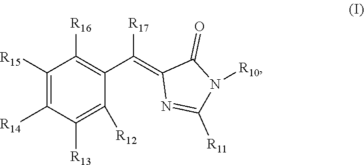
wherein:
R₁₀, R₁₁, and R₁₇ are each independently selected from the group consisting of hydrogen, deuterium, and optionally substituted C₁-C₆ alkyl;
R₁₂, R₁₃, R₁₄, R₁₅, and R₁₆ are each independently selected from the group consisting of hydrogen, deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;
each occurrence of R′ is independently selected from the group consisting of hydrogen, deuterium, hydroxy, C₁-C₆ alkyl, and C₁-C₆ alkoxy,
with the proviso that one or more of R₁₂, R₁₃, R₁₄, R₁₅, and R₁₆ is selected from the group consisting of OH, NHR′, and SH;
or a salt, solvate, stereoisomer, or geometric isomer thereof.

5. The compound of claim 4, wherein the biomarker is selected from the group consisting of hydrogen peroxide (H₂O₂), dimethylsulfide (H₂S), superoxide (O₂⁻), peroxynitrite (ONOO⁻), glutathione, hepatic lipase, cathepsin B, the caspase family, alkaline phosphatase, Cu(I), Fe(II), and Zn(II), and combinations thereof.

6. A method of detecting a disease or a disorder in a subject in need thereof, the method comprising the steps of:
(a) expressing an RNA sequence comprising an RNA aptamer in the subject;
(b) administering to the subject a compound of Formula (II): 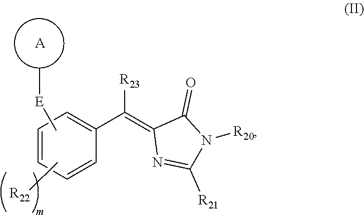
wherein:
R₂₀, R₂₁, and R₂₃ are each independently selected from the group consisting of hydrogen, deuterium, and optionally substituted C₁-C₆ alkyl;
each occurrence of R₂₂ is independently selected from the group consisting of deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;
each occurrence of R′ is independently selected from the group consisting of hydrogen, deuterium, hydroxy, C₁-C₆ alkyl, and C₁-C₆ alkoxy;
m is an integer from 0 to 4;
E is selected from the group consisting of —O—, —S—, and —NH—; 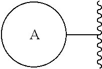
is selected from the group consisting of 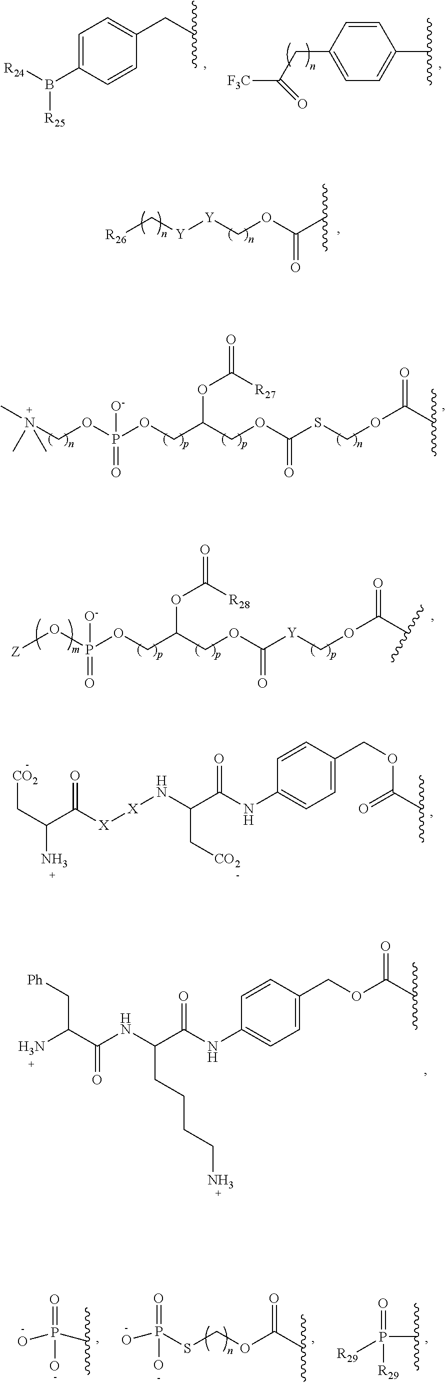 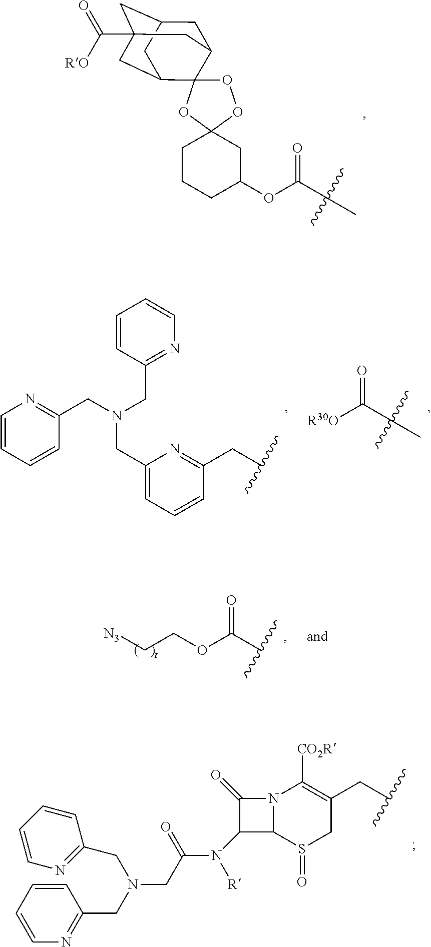
wherein:
m is 0 or 1;
each occurrence of n is independently 2 or 3;
each occurrence of p is independently 1, 2, or 3;
t is 1, 2, or 3;
R₂₄ and R₂₅ are each independently selected from the group consisting of hydroxy, C₁-C₆ alkyl, and C₁-C₆ alkoxy,
or R₂₄ and R₂₅ can combine with the atoms to which they are bound to form a 4-6 membered ring;
R₂₆ is hydroxy or 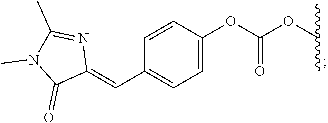 [SMILES: *OC(=O)Oc1ccc(/C=C2\N=C(C)N(C)C2=O)cc1]
R₂₇ and R₂₈ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;
each occurrence of R²⁹ is independently selected from the group consisting of optionally substituted C₁-C₆ alkyl, optionally substituted C₆-C₁₀ aryl, and optionally substituted C₂-C₁₀ heteroaryl;
R³⁰ is optionally substituted C₁-C₁₂ alkyl;
each occurrence of X is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;
Y is selected from the group consisting of —O—, —NH—, and —S—; and
Z is selected from the group consisting of choline, ethanolamine, serine, inositol, glycerol, phosphatidylcholine, lysophosphatidic acid, and glucose;
or a salt, solvate, stereoisomer, or geometric isomer thereof;
and
(c) detecting fluorescence emission associated with the RNA aptamer.

7. The method of claim 6, wherein the RNA aptamer is selected from the group consisting of Spinach aptamer, Baby Spinach aptamer, Corn aptamer, and Broccoli aptamer.

8. The method of claim 6, wherein the compound of Formula (II) is a compound of Formula (IIa): 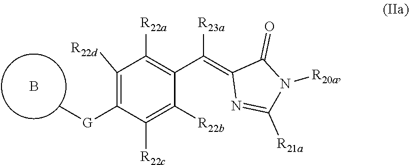
wherein:
G is selected from the group consisting of —O—, —S—, or —NH—;
R₂₀ₐ, R₂₁ₐ, and R₂₃ₐ are each independently selected from the group consisting of hydrogen, deuterium, and optionally substituted C₁-C₆ alkyl;
R^{22a}, R^{22b}, R^{22c}, and R^{22d} are each independently selected from the group consisting of hydrogen, deuterium, tritium, halogen, hydroxy, N(R″)(R″), SR″, sulfide, thiolactone, S(═O)₂OR″, S(═O)R″, cyano, C₁-C₆ alkyl, C₂-C₆ alkenyl, C₂-C₆ alkynyl, nitro, C(═O)N(R″)(R″), P(═O)(OR″)₂, PR″₃, C₆-C₁₂ aryl, C₄-C₁₀ heteroaryl, C₁-C₆ alkoxy, optionally substituted boron, optionally substituted silicon, transition metal, C(═O)OR″, and C(═O)R″;
each occurrence of R″ is independently selected from the group consisting of hydrogen, deuterium, hydroxy, C₁-C₆ alkyl, and C₁-C₆ alkoxy; 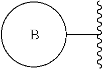
is selected from the group consisting of 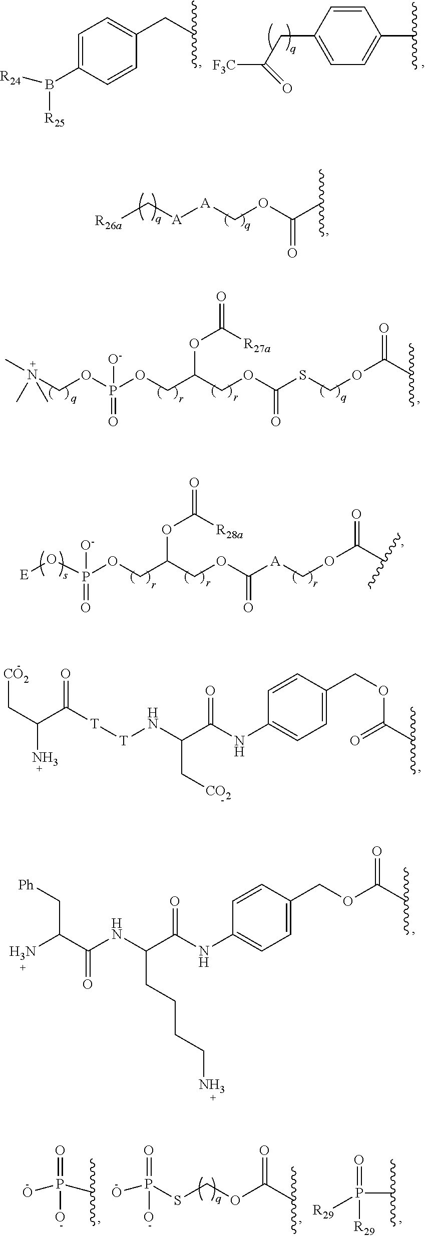 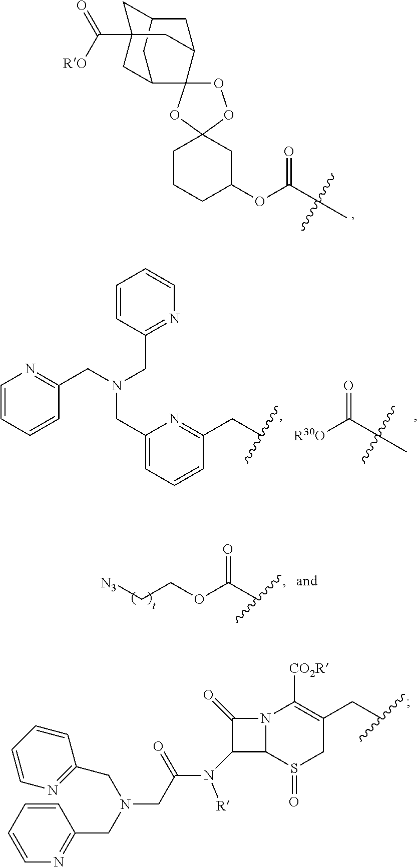
wherein:
each occurrence of q is independently 2 or 3;
each occurrence of r is independently 1, 2, or 3;
s is 0 or 1;
R₂₄ₐ and R₂₅ₐ are each independently selected from the group consisting of hydroxy, C₁-C₆ alkyl, and C₁-C₆ alkoxy,
or R₂₄ₐ and R₂₅ₐ can combine with the atoms to which they are bound to form a 4-6 membered ring;
R₂₆ₐ is hydroxy or 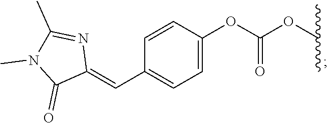 [SMILES: *OC(=O)Oc1ccc(/C=C2\N=C(C)N(C)C2=O)cc1]
R₂₇ₐ and R₂₈ₐ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;
A is selected from the group consisting of —O—, —NH—, and —S—;
E is selected from the group consisting of choline, ethanolamine, serine, inositol, glycerol, phosphatidylcholine, lysophosphatidic acid, and glucose; and
each occurrence of T is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and selenocysteine;
or a salt, solvate, stereoisomer, or geometric isomer thereof.

9. The method of claim 6, wherein the compound is selected from the group consisting of: 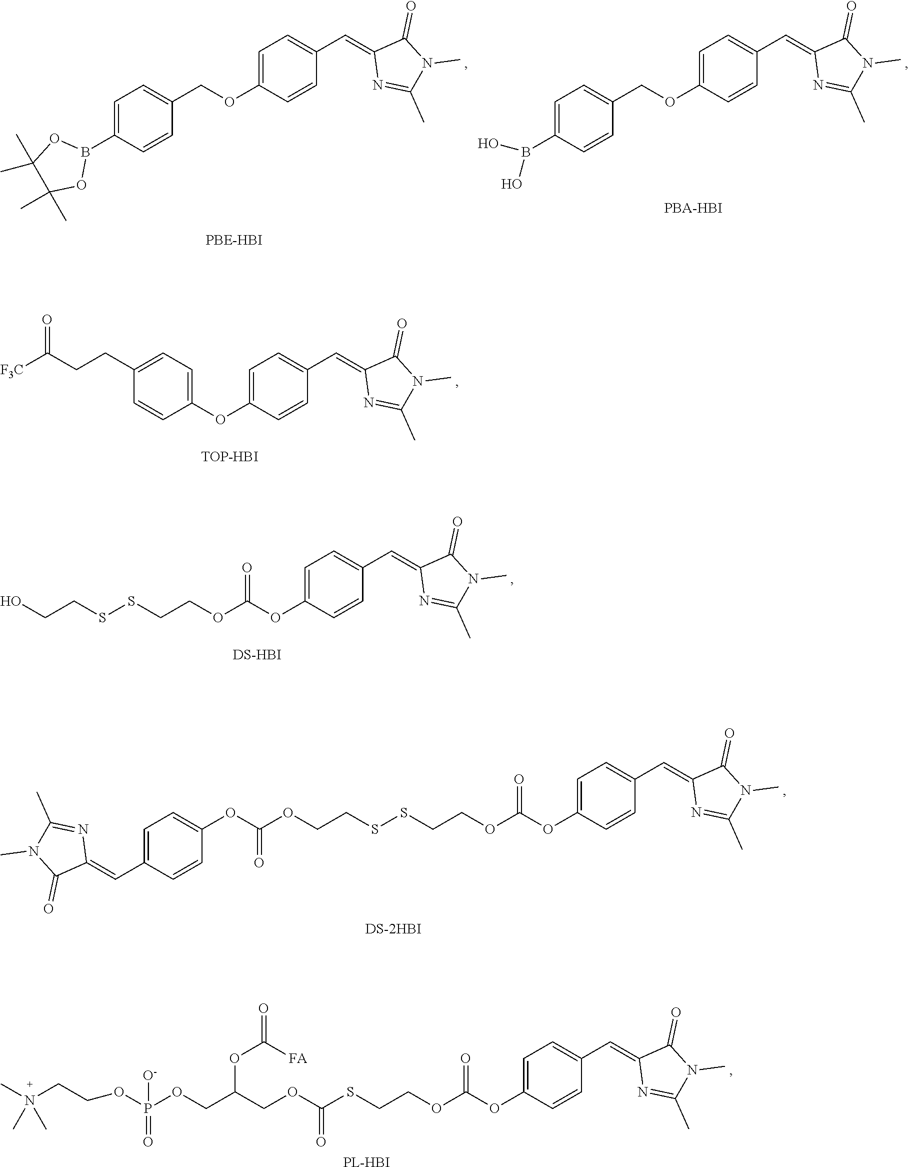 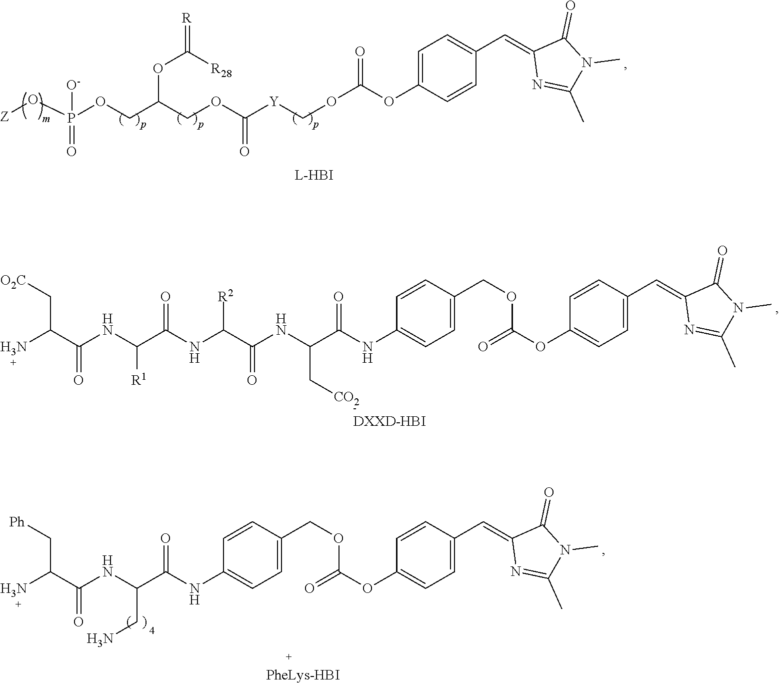 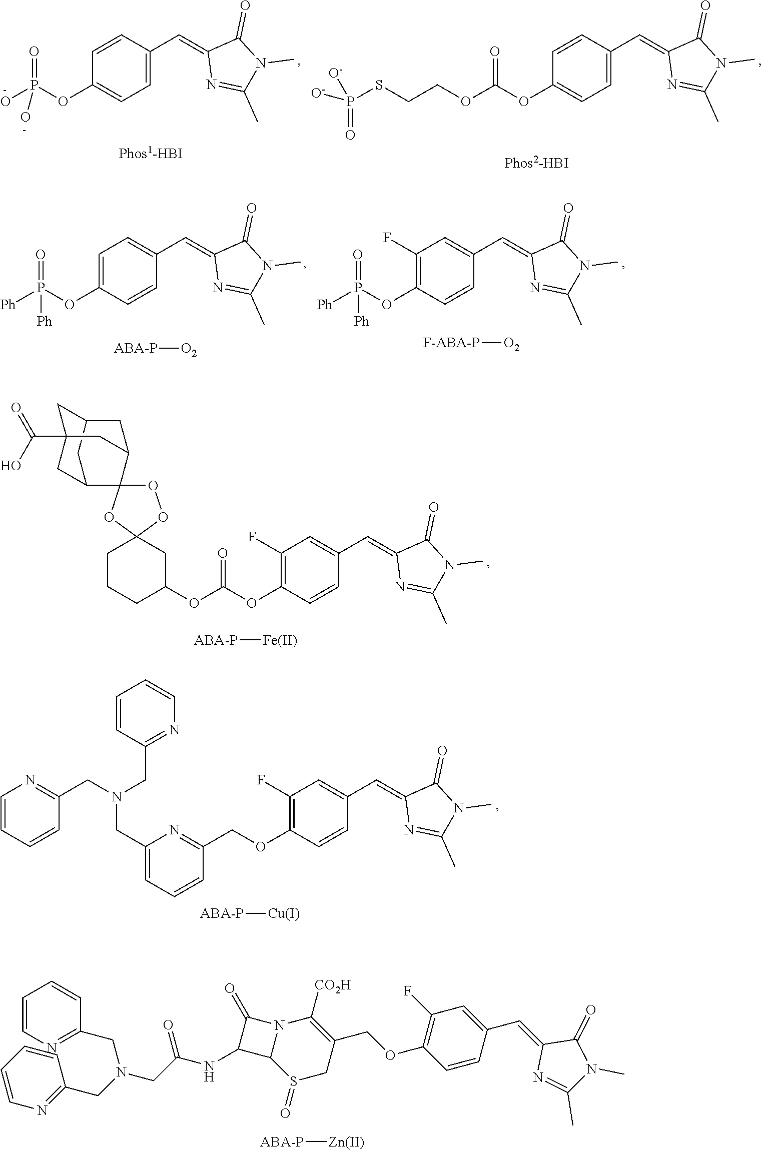 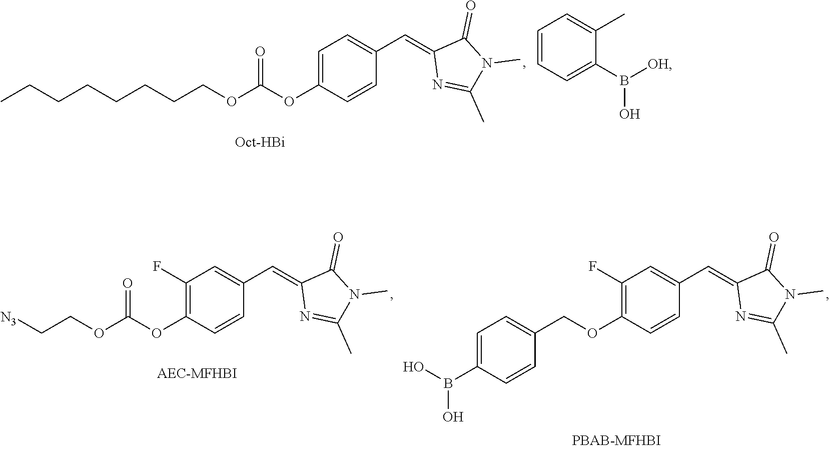
and combinations thereof,
wherein R¹ and R² are each independently selected from the group consisting of hydrogen, CH₃, CH₂—OH, and CH(OH)—CH₃.

10. The method of claim 6, wherein administering to the subject a compound of Formula (II) further comprises deprotecting the compound of Formula (II) via a reaction with a biomarker, producing a fluorogenic ligand.

11. The method of claim 10, wherein the biomarker is selected from the group consisting of hydrogen peroxide, dimethylsulfide, superoxide, hydroxyl radical, hydroxide anion, peroxynitrite, nitrogen dioxide, nitrosoperoxycarbonate, dinitrogen trioxide, aldehyde, glutathione, glutathione-synthesizing enzymes, lipases, cathepsin B, the caspase family, acid phosphatase, alkaline phosphatase, Cu(I), Fe(II), and Zn(II), and combinations thereof.

12. The method of claim 10, wherein the fluorogenic ligand is a compound of Formula (I): 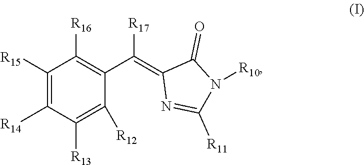
wherein:
R₁₀, R₁₁, and R₁₇ are each independently selected from the group consisting of hydrogen, deuterium, and optionally substituted C₁-C₆ alkyl;
R₁₂, R₁₃, R₁₄, R₁₅, and R₁₆ are each independently selected from the group consisting of hydrogen, deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;
each occurrence of R′ is independently selected from the group consisting of hydrogen, deuterium, hydroxy, C₁-C₆ alkyl, and C₁-C₆ alkoxy,
with the proviso that one or more of R₁₂, R₁₃, R₁₄, R₁₅, and R₁₆ is selected from the group consisting of OH, NHR′, and SH;
or a salt, solvate, stereoisomer, or geometric isomer thereof.

13. The method of claim 10, wherein deprotecting the compound of Formula (II) via a reaction with a biomarker further comprises binding the fluorogenic ligand to the RNA aptamer.

14. The method of claim 13, wherein binding the fluorogenic ligand to the RNA aptamer leads to fluorescence emission associated with the aptamer.

15. The method of claim 14, wherein the fluorescence emission associated with the aptamer indicates that the subject has a disease or disorder associated with the biomarker.

16. A method of detecting a disease or a disorder in a biological sample, the method comprising:
(a) providing a chip comprising a grafted RNA aptamer;
(b) contacting the chip with a biological sample;
(c) contacting the biological sample with a compound of Formula (II): 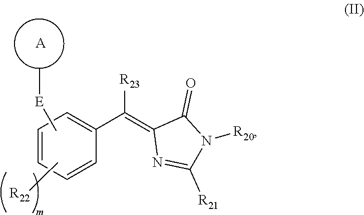
wherein:
R₂₀, R₂₁, and R₂₃ are each independently selected from the group consisting of hydrogen, deuterium, and optionally substituted C₁-C₆ alkyl;
each occurrence of R₂₂ is independently selected from the group consisting of deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;
each occurrence of R′ is independently selected from the group consisting of hydrogen, deuterium, hydroxy, C₁-C₆ alkyl, and C₁-C₆ alkoxy;
m is an integer from 0 to 4;
E is selected from the group consisting of —O—, —S—, and —NH—; 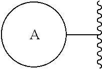
is selected from the group consisting of 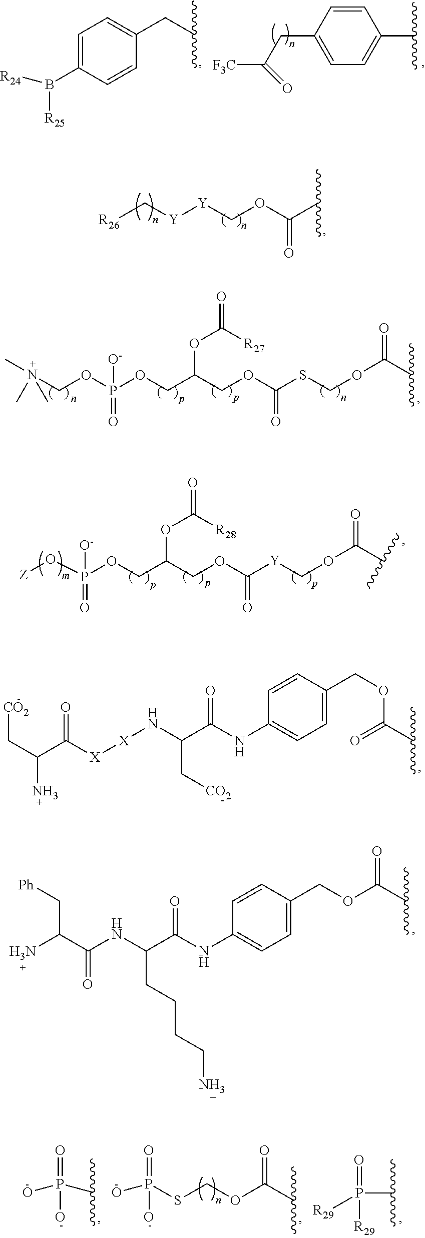 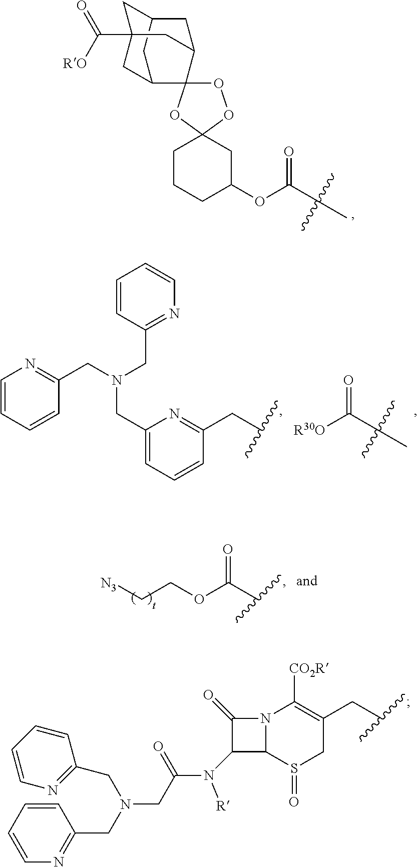
wherein:
m is 0 or 1;
each occurrence of n is independently 2 or 3;
each occurrence of p is independently 1, 2, or 3;
t is 1, 2, or 3;
R₂₄ and R₂₅ are each independently selected from the group consisting of hydroxy, C₁-C₆ alkyl, and C₁-C₆ alkoxy,
or R₂₄ and R₂₅ can combine with the atoms to which they are bound to form a 4-6 membered ring;
R₂₆ is hydroxy or 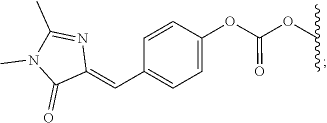 [SMILES: *OC(=O)Oc1ccc(/C=C2\N=C(C)N(C)C2=O)cc1]
R₂₇ and R₂₈ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;
each occurrence of R²⁹ is independently selected from the group consisting of optionally substituted C₁-C₆ alkyl, optionally substituted C₆-C₁₀ aryl, and optionally substituted C₂-C₁₀ heteroaryl;
R³⁰ is optionally substituted C₁-C₁₂ alkyl;
each occurrence of X is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;
Y is selected from the group consisting of —O—, —NH—, and —S—; and
Z is selected from the group consisting of choline, ethanolamine, serine, inositol, glycerol, phosphatidylcholine, lysophosphatidic acid, and glucose;
or a salt, solvate, stereoisomer, or geometric isomer thereof;
(d) rinsing the chip; and
(e) detecting fluorescence emission from the RNA aptamer.

17. The method of claim 16, wherein the RNA aptamer is Spinach aptamer, Baby Spinach aptamer, Corn aptamer, or Broccoli aptamer.

18. The method of claim 16, wherein the compound of Formula (II) is a compound of Formula (IIa): 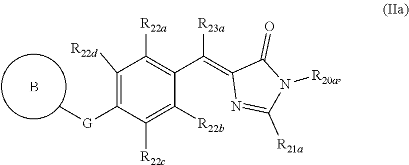
wherein:
G is selected from the group consisting of —O—, —S—, or —NH—;
R₂₀ₐ, R₂₁ₐ, and R₂₃ₐ are each independently selected from the group consisting of hydrogen, deuterium, and optionally substituted C₁-C₆ alkyl;
R^{22a}, R^{22b}, R^{22c}, and R^{22d} are each independently selected from the group consisting of hydrogen, deuterium, tritium, halogen, hydroxy, N(R″)(R″), SR″, sulfide, thiolactone, S(═O)₂OR″, S(═O)R″, cyano, C₁-C₆ alkyl, C₂-C₆ alkenyl, C₂-C₆ alkynyl, nitro, C(═O)N(R″)(R″), P(═O)(OR″)₂, PR″₃, C₆-C₁₂ aryl, C₄-C₁₀ heteroaryl, C₁-C₆ alkoxy, optionally substituted boron, optionally substituted silicon, transition metal, C(═O)OR″, and C(═O)R″;
each occurrence of R″ is independently selected from the group consisting of hydrogen, deuterium, hydroxy, C₁-C₆ alkyl, and C₁-C₆ alkoxy; 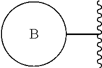
is selected from the group consisting of 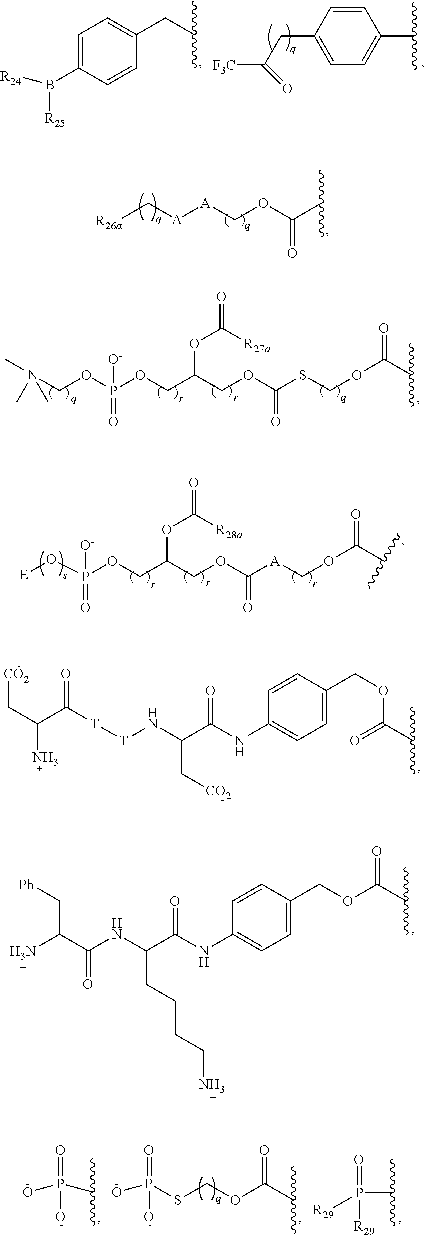 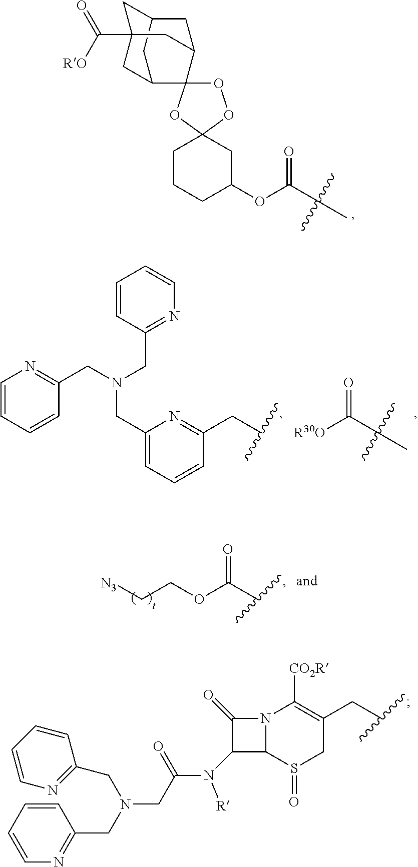
wherein:
each occurrence of q is independently 2 or 3;
each occurrence of r is independently 1, 2, or 3;
s is 0 or 1;
R₂₄ₐ and R₂₅ₐ are each independently selected from the group consisting of hydroxy, C₁-C₆ alkyl, and C₁-C₆ alkoxy,
or R₂₄ₐ and R₂₅ₐ can combine with the atoms to which they are bound to form a 4-6 membered ring;
R₂₆ₐ is hydroxy or 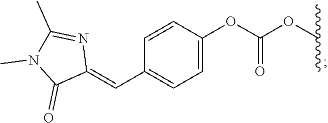 [SMILES: *OC(=O)Oc1ccc(/C=C2\N=C(C)N(C)C2=O)cc1]
R₂₇ₐ and R₂₈ₐ are each independently selected from the group consisting of C₄-C₂₈ alkyl, C₄-C₂₈ alkenyl, steroid family lipids, sex hormones or androgens, glucocorticoids, mineralocorticoids, dexamethasone, and combinations thereof;
A is selected from the group consisting of —O—, —NH—, and —S—;
E is selected from the group consisting of choline, ethanolamine, serine, inositol, glycerol, phosphatidylcholine, lysophosphatidic acid, and glucose; and
each occurrence of T is independently an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and selenocysteine;
or a salt, solvate, stereoisomer, or geometric isomer thereof.

19. The method of claim 16, wherein the compound is selected from the group consisting of: 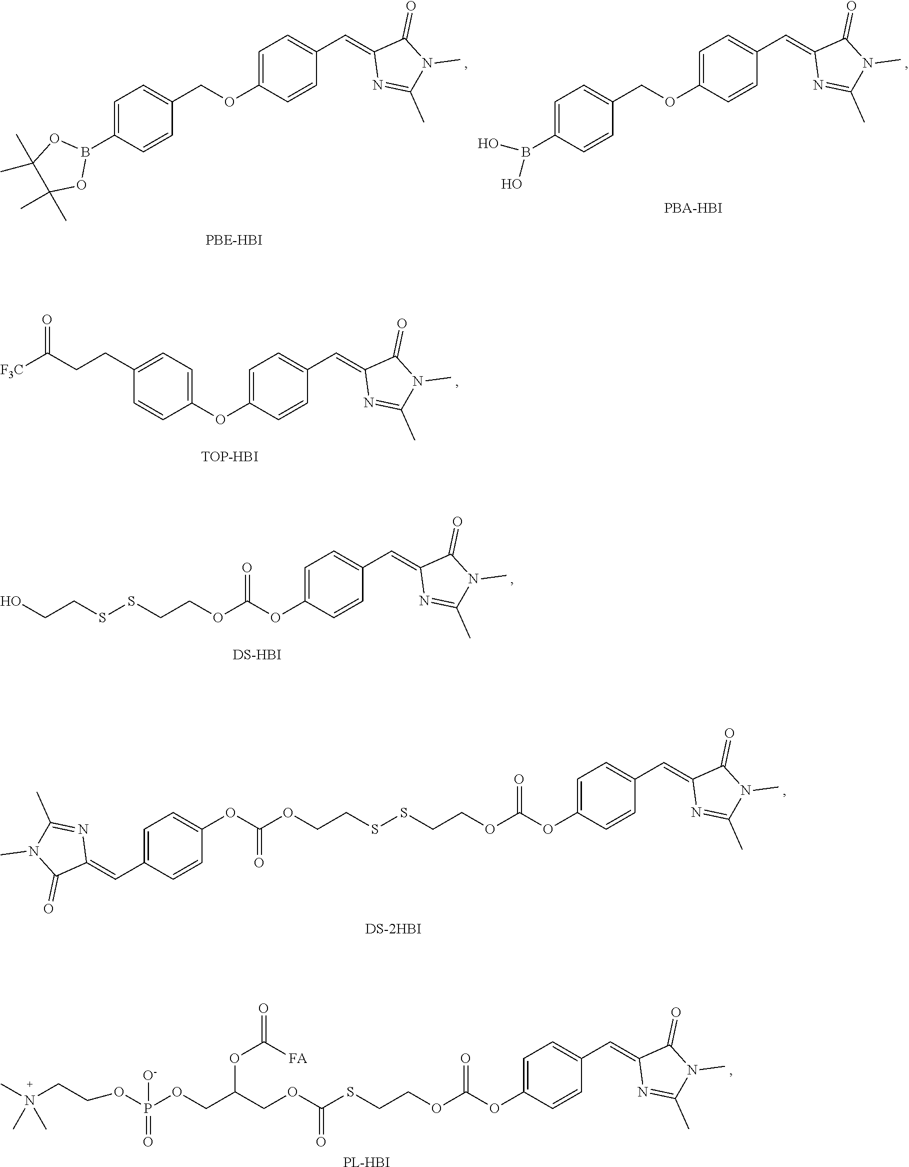 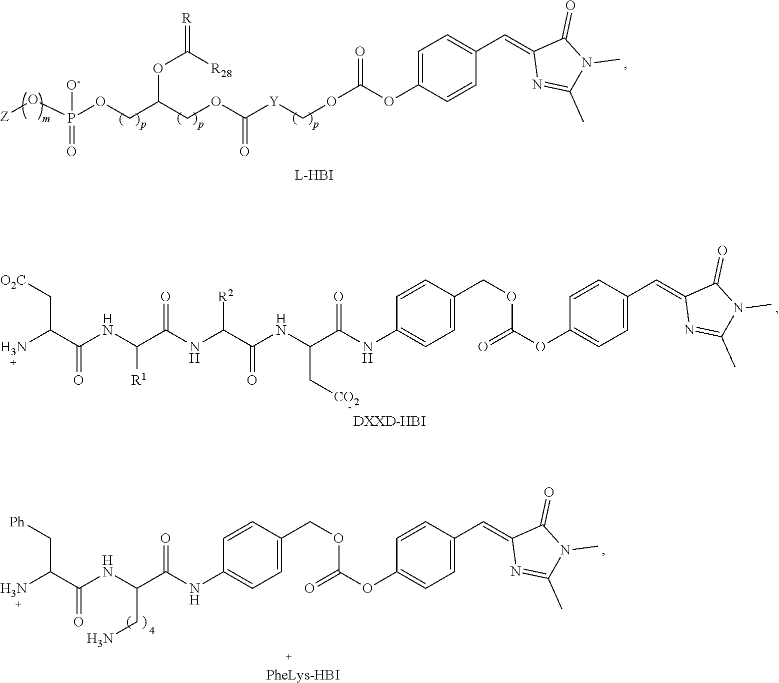 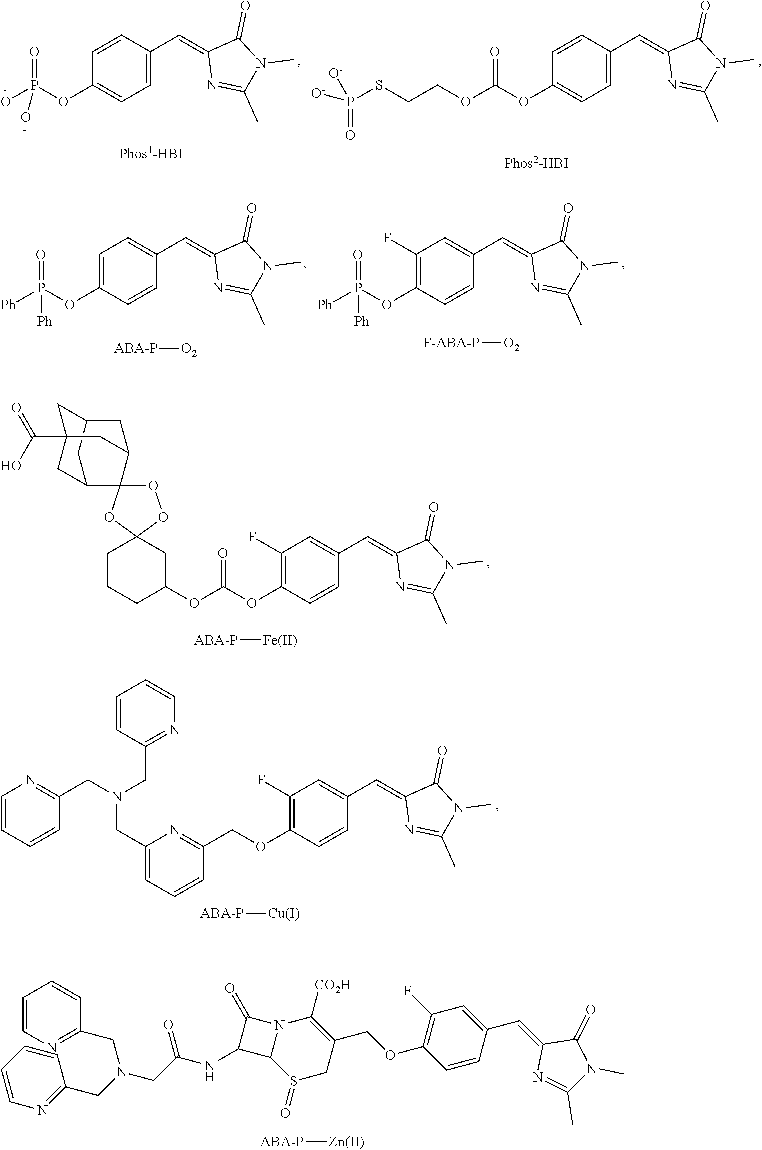 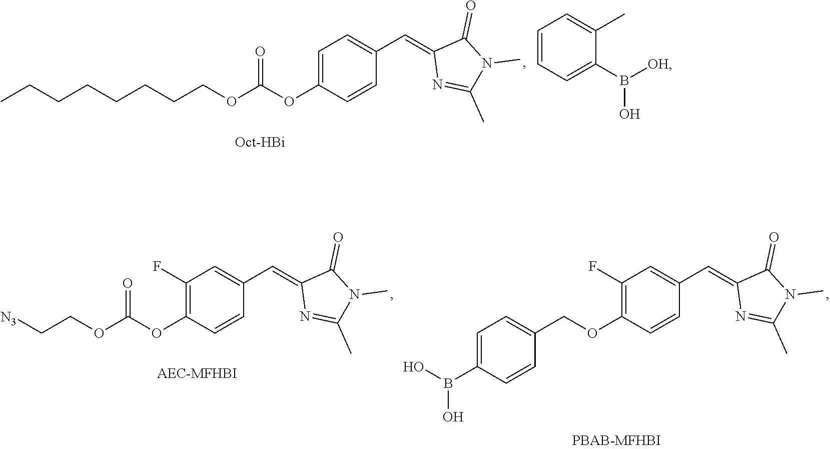
and combinations thereof;
wherein R¹ and R² are each independently selected from the group consisting of hydrogen, CH₃, CH₂—OH, and CH(OH)—CH₃.

20. The method of claim 16, wherein contacting the biological sample with a compound of Formula (II) further comprises deprotecting the compound of Formula (II) via a reaction with a biomarker present in the biological sample, producing a fluorogenic ligand, wherein the fluorogenic ligand is optionally a compound of Formula (I): 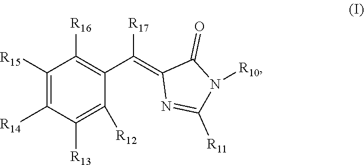
wherein:
R₁₀, R₁₁, and R₁₇ are each independently selected from the group consisting of hydrogen, deuterium, and optionally substituted C₁-C₆ alkyl;
R₁₂, R₁₃, R₁₄, R₁₅, and R₁₆ are each independently selected from the group consisting of hydrogen, deuterium, tritium, halogen, hydroxy, N(R′)(R′), SR′, sulfide, thiolactone, S(═O)₂OR′, S(═O)R′, cyano, C₁-C₆ alkyl, C₂-C₆ alkenyl, C₂-C₆ alkynyl, nitro, C(═O)N(R′)(R′), P(═O)(OR′)₂, PR′₃, C₆-C₁₂ aryl, C₄-C₁₀ heteroaryl, C₁-C₆ alkoxy, optionally substituted boron, optionally substituted silicon, transition metal, C(═O)OR′, and C(═O)R′;
each occurrence of R′ is independently selected from the group consisting of hydrogen, deuterium, hydroxy, C₁-C₆ alkyl, and C₁-C₆ alkoxy,
with the proviso that one or more of R₁₂, R₁₃, R₁₄, R₁₅, and R₁₆ is selected from the group consisting of OH, NHR′, and SH;
or a salt, solvate, stereoisomer, or geometric isomer thereof.
